(19) United States
(12) Patent Application Publication
(10) Pub. No.: US 2004/0023242 A1
(43) Pub. Date: Feb. 5, 2004

(54) Human kinases

(76) Inventors: Henry Yue, Sunnyvale, CA (US); Preeti G. Lal, Santa Clara, CA (US); Olga Bandman, Mountain View, CA (US); Mark L Borowsky, Northampton, MA (US); Janice K. Au-Young, Brisbane, CA (US); Yan Lu, Mountain View, CA (US); Ameena R. Gandhi, San Francisco, CA (US); Catherine M. Tribouley, San Francisco, CA (US); Narinder K. Chawla, Union City, CA (US); Monique G. Yao, Carmel, IN (US); Dyung Aina M. Lu, San Jose, CA (US); Sara R. Greenwald, San Francisco, CA (US); Jayalaxmi Ramkumar, Fremont, CA (US); Jennifer A. Griffin, Fremont, CA (US); Liam Kearney, San Jose, CA (US); Neil Burford, Durham, CT (US); Danniel B. Nguyen, San Jose, CA (US); Y. Tom Tang, San Jose, CA (US); Mariah R Baughn, San Leandro, CA (US); Ann He, San Jose, CA (US); Michael B. Thornton, Oakland, CA (US); April J.A. Hafalia, Daly City, CA (US); Chandra S. Arvizu, San Jose, CA (US); Rajagopal Gururajan, San Jose, CA (US); Terence P. Lo, Foster City, CA (US); Farrah A. Khan, Des Plaines, IL (US); Shirley A. Recipon, San Francisco, CA (US); Yalda Azimzai, Oakland, CA (US); Jennifer L. Policky, San Jose, CA (US); Li Ding, Creve Coeur, MO (US); Megan E. Grether, San Francisco, CA (US); Vicki S. Elliott, San Jose, CA (US); Kavitha Thangavelu, Sunnyvale, CA (US); Sajeev Batra, Oakland, CA (US); Craig H. Ison, San Jose, CA (US)

Correspondence Address: INCYTE CORPORATION (formerly known as Incyte, Genomics, Inc.), 3160 PORTER DRIVE, PALO ALTO, CA, 94304, US

(21) Appl. No.: 10/311,034

(22) PCT Filed: Jun. 14, 2001
(86) PCT No.: PCT/US01/19444

(51) Int. Cl. C12Q 1/68; G01N 33/53; C12N 9/12; C07H 21/04; C07K 16/40

(52) U.S. Cl. 435/6; 435/69.1; 435/194; 435/320.1; 435/325; 530/388.26; 536/23.2; 435/7.1

(57) ABSTRACT

The invention provides human kinases (PKIN) and polynucleotides which identify and encode PKIN. The invention also provides expression vectors, host cells, antibodies, agonists, and antagonists. The invention also provides methods for diagnosing, treating or prevention disorders associated with aberrant expression of PKIN.

### TECHNICAL FIELD

This invention relates to nucleic acid and amino acid sequences of human kinases and to the use of these sequences in the diagnosis, treatment, and prevention of cancer, immune disorders, disorders affecting growth and development, cardiovascular diseases, and lipid disorders, and in the assessment of the effects of exogenous compounds on the expression of nucleic acid and amino acid sequences of human kinases.

### BACKGROUND OF THE INVENTION

Kinases comprise the largest known enzyme superfamily and vary widely in their target molecules. Kinases catalyze the transfer of high energy phosphate groups from a phosphate donor to a phosphate acceptor. Nucleotides usually serve as the phosphate donor in these reactions, with most kinases utilizing adenosine triphosphate (ATP). The phosphate acceptor can be any of a variety of molecules, including nucleosides, nucleotides, lipids, carbohydrates, and proteins. Proteins are phosphorylated on hydroxyamino acids. Addition of a phosphate group alters the local charge on the acceptor molecule, causing internal conformational changes and potentially influencing intermolecular contacts. Reversible protein phosphorylation is the primary method for regulating protein activity in eukaryotic cells. In general, proteins are activated by phosphorylation in response to extracellular signals such as hormones, neurotransmitters, and growth and differentiation factors. The activated proteins initiate the cell's intracellular response by way of intracellular signaling pathways and second messenger molecules such as cyclic nucleotides, calcium-calmodulin, inositol, and various mitogens, that regulate protein phosphorylation.

Kinases are involved in all aspects of a cell's function, from basic metabolic processes, such as glycolysis, to cell-cycle regulation, differentiation, and communication with the extracellular environment through signal transduction cascades. Inappropriate phosphorylation of proteins in cells has been linked to changes in cell cycle progression and cell differentiation. Changes in the cell cycle have been linked to induction of apoptosis or cancer. Changes in cell differentiation have been linked to diseases and disorders of the reproductive system, immune system, and skeletal muscle.

There are two classes of protein kinases. One class, protein tyrosine kinases (PTKs), phosphorylates tyrosine residues, and the other class, protein serine/threonine kinases (STKs), phosphorylates serine and threonine residues. Some PTKs and STKs possess structural characteristics of both families and have dual specificity for both tyrosine and serine/threonine residues. Almost all kinases contain a conserved 250-300 amino acid catalytic domain containing specific residues and sequence motifs characteristic of the kinase family. The protein kinase catalytic domain can be further divided into 11 subdomains. N-terminal subdomains I-IV fold into a two-lobed structure which binds and orients the ATP donor molecule, and subdomain V spans the two lobes. C-terminal subdomains VI-XI bind the protein substrate and transfer the gamma phosphate from ATP to the hydroxyl group of a tyrosine, serine, or threonine residue. Each of the 11 subdomains contains specific catalytic residues or amino acid motifs characteristic of that subdomain. For example, subdomain I contains an 8-amino acid glycine-rich ATP binding consensus motif, subdomain II contains a critical lysine residue required for maximal catalytic activity, and subdomains VI through IX comprise the highly conserved catalytic core. PTKs and STKs also contain distinct sequence motifs in subdomains VI and VIII which may confer hydroxyamino acid specificity.

In addition, kinases may also be classified by additional amino acid sequences, generally between 5 and 100 residues, which either flank or occur within the kinase domain. These additional amino acid sequences regulate kinase activity and determine substrate specificity. (Reviewed in Hardie, G. and S. Hanks (1995) The Protein Kinase Facts Book Vol I, pp.17-20 Academic Press, San Diego Calif.). In particular, two protein kinase signature sequences have been identified in the kinase domain, the first containing an active site lysine residue involved in ATP binding, and the second containing an aspartate residue important for catalytic activity. If a protein analyzed includes the two protein kinase signatures, the probability of that protein being a protein kinase is close to 100% (PROSITE: PDOC00100, November 1995).

Protein Tyrosine Kinases

Protein tyrosine kinases (PTKs) may be classified as either transmembrane, receptor PTKs or nontransmembrane, nonreceptor PTK proteins. Transmembrane tyrosine kinases function as receptors for most growth factors. Growth factors bind to the receptor tyrosine kinase (RTK), which causes the receptor to phosphorylate itself (autophosphorylation) and specific intracellular second messenger proteins. Growth factors (GF) that associate with receptor PTKs include epidermal GF, platelet-derived GF, fibroblast GF, hepatocyte GF, insulin and insulin-like GFs, nerve GF, vascular endothelial GF, and macrophage colony stimulating factor.

Nontransmembrane, nonreceptor PTKs lack transmembrane regions and, instead, form signaling complexes with the cytosolic domains of plasma membrane receptors. Receptors that function through non-receptor PTKs include those for cytokines and hormones (growth hormone and prolactin), and antigen-specific receptors on T and B lymphocytes.

Many PTKs were first identified as oncogene products in cancer cells in which PTK activation was no longer subject to normal cellular controls. In fact, about one third of the known oncogenes encode PTKs. Furthermore, cellular transformation (oncogenesis) is often accompanied by increased tyrosine phosphorylation activity (Charbonneau, H. and N. K. Tonks (1992) Annu. Rev. Cell Biol. 8:463-493). Regulation of PTK activity may therefore be an important strategy in controlling some types of cancer.

Protein Serine/Threonine Kinases

Protein serine/threonine kinases (STKs) are nontransmembrane proteins. A subclass of STKs are known as ERKs (extracellular signal regulated kinases) or MAPs (mitogen-activated protein kinases) and are activated after cell stimulation by a variety of hormones and growth factors. Cell stimulation induces a signaling cascade leading to phosphorylation of MEK (MAP/ERK kinase) which, in turn, activates ERK via serine and threonine phosphorylation. A varied number of proteins represent the downstream effectors for the active ERK and implicate it in the control of cell proliferation and differentiation, as well as regulation of the cytoskeleton. Activation of ERK is normally transient, and cells possess dual specificity phosphatases that are responsible for its down-regulation. Also, numerous studies have shown that elevated ERK activity is associated with some cancers. Other STKs include the second messenger dependent protein kinases such as the cyclic-AMP dependent protein kinases (PKA), calcium-calmodulin (CaM) dependent protein kinases, and the mitogen-activated protein kinases (MAP); the cyclin-dependent protein kinases; checkpoint and cell cycle kinases; Numb-associated kinase (Nak); human Fused (hFu); proliferation-related kinases; 5′-AMP-activated protein kinases; and kinases involved in apoptosis.

The second messenger dependent protein kinases primarily mediate the effects of second messengers such as cyclic AMP (cAMP), cyclic GMP, inositol triphosphate, phosphatidylinositol, 3,4,5-triphosphate, cyclic ADP ribose, arachidonic acid, diacylglycerol and calcium-calmodulin. The PKAs are involved in mediating hormone-induced cellular responses and are activated by cAMP produced within the cell in response to hormone stimulation. cAMP is an intracellular mediator of hormone action in all animal cells that have been studied. Hormone-induced cellular responses include thyroid hormone secretion, cortisol secretion, progesterone secretion, glycogen breakdown, bone resorption, and regulation of heart rate and force of heart muscle contraction. PKA is found in all animal cells and is thought to account for the effects of cAMP in most of these cells. Altered PKA expression is implicated in a variety of disorders and diseases including cancer, thyroid disorders, diabetes, atherosclerosis, and cardiovascular disease (Isselbacher, K. J. et al. (1994) Harrison's Principles of Internal Medicine, McGraw-Hill, New York NY, pp. 416-431, 1887).

The casein kinase I (CKI) gene family is another subfamily of serine/threonine protein kinases. This continuously expanding group of kinases have been implicated in the regulation of numerous cytoplasmic and nuclear processes, including cell metabolism, and DNA replication and repair. CKI enzymes are present in the membranes, nucleus, cytoplasm and cytoskeleton of eukaryotic cells, and on the mitotic spindles of mammalian cells (Fish, K. J. et al. (1995) J. Biol. Chen. 270:14875-14883).

The CKI family members all have a short amino-terminal domain of 9-76 amino acids, a highly conserved kinase domain of 284 amino acids, and a variable carboxyl-terminal domain that ranges from 24 to over 200 amino acids in length (Cegielska, A. et al. (1998) J. Biol. Chem. 273:1357-1364). The CKI family is comprised of highly related proteins, as seen by the identification of isoforms of casein kinase I from a variety of sources. There are at least five mammalian isoforms, α, β, δ, and ε. Fish et al., identified CKI-epsilon from a human placenta cDNA library. It is a basic protein of 416 amino acids and is closest to CKI-delta. Through recombinant expression, it was determined to phosphorylate known CKI substrates and was inhibited by the CKI-specific inhibitor CKI-7. The human gene for CKI-epsilon was able to rescue yeast with a slow-growth phenotype caused by deletion of the yeast CKI locus, HRR250 (Fish et al., supra).

The mammalian circadian mutation tau was found to be a semidominant autosomal allele of CKI-epsilon that markedly shortens period length of circadian rhythms in Syrian hamsters. The tau locus is encoded by casein kinase I-epsilon, which is also a homolog of the Drosophila circadian gene double-time. Studies of both the wildtype and tau mutant CKI-epsilon enzyme indicated that the mutant enzyme has a noticeable reduction in the maximum velocity and autophosphorylation state. Further, in vitro, CKI-epsilon is able to interact with mammalian PERIOD proteins, while the mutant enzyme is deficient in its ability to phosphorylate PERIOD. Lowrey et al., have proposed that CKI-epsilon plays a major role in delaying the negative feedback signal within the transcription-translation-based autoregulatory loop that composes the core of the circadian mechanism. Therefore the CKI-epsilon enzyme is an ideal target for pharmaceutical compounds influencing circadian rhythms, jet-lag and sleep, in addition to other physiologic and metabolic processes under circadian regulation (Lowrey, P. L. et al. (2000) Science 288:483-491).

Homeodomain-interacting protein kinases (HIPKs) are serine/threonine kinases and novel members of the DYRK kinase subfamily (Hofmann, T. G. et al. (2000) Biochimie 82:1123-1127). HIPKs contain a conserved protein kinase domain separated from a domain that interacts with homeoproteins. HIPKs are nuclear kinases, and HIPK2 is highly expressed in neuronal tissue (Kim, Y. H. et al. (1998) J. Biol. Chem. 273:25875-25879; Wang, Y. et al. (2001) Biochim. Biophys. Acta 1518:168-172). HIPKs act as corepressors for homeodomian transcription factors. This corepressor activity is seen in posttranslational modifications such as ubiquitination and phosphorylation, each of which are important in the regulation of cellular protein function (Kim, Y. H. et al. (1999) Proc. Natl. Acad. Sci. USA 96:12350-12355).

Calcium-Calmodulin Dependent Protein Kinases

Calcium-calmodulin dependent (CaM) kinases are involved in regulation of smooth muscle contraction, glycogen breakdown (phosphorylase kinase), and neurotransmission (CaM kinase I and CaM kinase II). CaM dependent protein kinases are activated by calmodulin, an intracellular calcium receptor, in response to the concentration of free calcium in the cell. Many CaM kinases are also activated by phosphorylation. Some CaM kinases are also activated by autophosphorylation or by other regulatory kinases. CaM kinase I phosphorylates a variety of substrates including the neurotransmitter-related proteins synapsin I and II, the gene transcription regulator, CREB, and the cystic fibrosis conductance regulator protein, CFTR (Haribabu, B. et al. (1995) EMBO J. 14:3679-3686). CaM kinase II also phosphorylates synapsin at different sites and controls the synthesis of catecholamines in the brain through phosphorylation and activation of tyrosine hydroxylase. CaM kinase II controls the synthesis of catecholamines and seratonin, through phosphorylation/activation of tyrosine hydroxylase and tryptophan hydroxylase, respectively (Fujisawa, H. (1990) BioEssays 12:27-29). The mRNA encoding a calmodulin-binding protein kinase-like protein was found to be enriched in mammalian forebrain. This protein is associated with vesicles in both axons and dendrites and accumulates largely postnatally. The amino acid sequence of this protein is similar to CaM-dependent STKs, and the protein binds calmodulin in the presence of calcium (Godbout, M. et al. (1994) J. Neurosci. 14:1-13).

Mitogen-Activated Protein Kinases

The mitogen-activated protein kinases (MAP) which mediate signal transduction from the cell surface to the nucleus via phosphorylation cascades are another STK family that regulates intracellular signaling pathways. Several subgroups have been identified, and each manifests different substrate specificities and responds to distinct extracellular stimuli (Egan, S. E. and R. A. Weinberg (1993) Nature 365:781-783). MAP kinase signaling pathways are present in mammalian cells as well as in yeast. The extracellular stimuli which activate MAP kinase pathways include epidermal growth factor (EGF), ultraviolet light, hyperosmolar medium, heat shock, endotoxic lipopolysaccharide (LPS), and pro-inflammatory cytokines such as tumor necrosis factor (TNF) and interleukin-1 (IL-1). Altered MAP kinase expression is implicated in a variety of disease conditions including cancer, inflammation, immune disorders, and disorders affecting growth and development.

Cyclin-Dependent Protein Kinases

The cyclin-dependent protein kinases (CDKs) are STKs that control the progression of cells through the cell cycle. The entry and exit of a cell from mitosis are regulated by the synthesis and destruction of a family of activating proteins called cyclins. Cyclins are small regulatory proteins that bind to and activate CDKs, which then phosphorylate and activate selected proteins involved in the mitotic process. CDKs are unique in that they require multiple inputs to become activated. In addition to cyclin binding, CDK activation requires the phosphorylation of a specific threonine residue and the dephosphorylation of a specific tyrosine residue on the CDK.

Another family of STKs associated with the cell cycle are the NIMA (never in mitosis)-related kinases (Neks). Both CDKs and Neks are involved in duplication, maturation, and separation of the microtubule organizing center, the centrosome, in animal cells (Fry, A. M. et al. (1998) EMBO J. 17:470481).

Checkpoint and Cell Cycle Kinases

In the process of cell division, the order and timing of cell cycle transitions are under control of cell cycle checkpoints, which ensure that critical events such as DNA replication and chromosome segregation are carried out with precision If DNA is damaged, e.g. by radiation, a checkpoint pathway is activated that arrests the cell cycle to provide time for repair. If the damage is extensive, apoptosis is induced. In the absence of such checkpoints, the damaged DNA is inherited by aberrant cells which may cause proliferative disorders such as cancer. Protein kinases play an important role in this process. For example, a specific kinase, checkpoint kinase 1 (Chk1), has been identified in yeast and mammals, and is activated by DNA damage in yeast. Activation of Chk1 leads to the arrest of the cell at the G2/M transition (Sanchez, Y. et al. (1997) Science 277:1497-1501). Specifically, Chk1 phosphorylates the cell division cycle phosphatase CDC25, inhibiting its normal function which is to dephosphorylate and activate the cyclin-dependent kinase Cdc2. Cdc2 activation controls the entry of cells into mitosis (Peng, C. -Y. et al. (1997) Science 277:1501-1505). Thus, activation of Chk1 prevents the damaged cell from entering mitosis. A similar deficiency in a checkpoint kinase, such as Chk1, may also contribute to cancer by failure to arrest cells with damaged DNA at other checkpoints such as G2/M.

Proliferation-Related Kinases

Proliferation-related kinase is a serum/cytokine inducible STK that is involved in regulation of the cell cycle and cell proliferation in human megakarocytic cells (Li, B. et al. (1996) J. Biol. Chem. 271:19402-19408). Proliferation-related kinase is related to the polo (derived from Drosophila polo gene) family of STKs implicated in cell division. Proliferation-related kinase is downregulated in lung tumor tissue and may be a proto-oncogene whose deregulated expression in normal tissue leads to oncogenic transformation

5 ′-AMP-Activated Protein Kinase

A ligand-activated STK protein kinase is 5′-AMP-activated protein kinase (AMPK) (Gao, G. et al. (1996) J. Biol Chem. 271:8675-8681). Mammalian AMPK is a regulator of fatty acid and sterol synthesis through phosphorylation of the enzymes acetyl-CoA carboxylase and hydroxymethylglutaryl-CoA reductase and mediates responses of these pathways to cellular stresses such as heat shock and depletion of glucose and ATP. AMPK is a heterotrimeric complex comprised of a catalytic alpha subunit and two non-catalytic beta and gamma subunits that are believed to regulate the activity of the alpha subunit. Subunits of AMPK have a much wider distribution in non-lipogenic tissues such as brain, heart, spleen, and lung than expected. This distribution suggests that its role may extend beyond regulation of lipid metabolism alone.

Kinases in Apoptosis

Apoptosis is a highly regulated signaling pathway leading to cell death that plays a crucial role in tissue development and homeostasis. Deregulation of this process is associated with the pathogenesis of a number of diseases including autoimmune disease, neurodegenerative disorders, and cancer. Various STKs play key roles in this process. ZIP kinase is an STK containing a C-terminal leucine zipper domain in addition to its N-terminal protein kinase domain. This C-terminal domain appears to mediate homodimerization and activation of the kinase as well as interactions with transcription factors such as activating transcription factor, ATF4, a member of the cyclic-AMP responsive element binding protein (ATF/CREB) family of transcriptional factors (Sanjo, H. et al. (1998) J. Biol. Chem. 273:29066-29071). DRAK1 and DRAK2 are STKs that share homology with the death-associated protein kinases (DAP kinases), known to function in interferon-γ induced apoptosis (Sanjo et al., supra). Like ZIP kinase, DAP kinases contain a C-terminal protein-protein interaction domain, in the form of ankyrin repeats, in addition to the N-terminal kinase domain. ZIP, DAP, and DRAK kinases induce morphological changes associated with apoptosis when transfected into NIH3T3 cells (Sanjo et al., supra). However, deletion of either the N-terminal kinase catalytic domain or the C-terminal domain of these proteins abolishes apoptosis activity, indicating that in addition to the kinase activity, activity in the C-terminal domain is also necessary for apoptosis, possibly as an interacting domain with a regulator or a specific substrate.

RICK is another STK recently identified as mediating a specific apoptotic pathway involving the death receptor, CD95 (Inohara, N. et al. (1998) J. Biol. Chem. 273:12296-12300). CD95 is a member of the tumor necrosis factor receptor superfamily and plays a critical role in the regulation and homeostasis of the immune system (Nagata, S. (1997) Cell 88:355-365). The CD95 receptor signaling pathway involves recruitment of various intracellular molecules to a receptor complex following ligand binding. This process includes recruitment of the cysteine protease caspase-8 which, in turn, activates a caspase cascade leading to cell death. RICK is composed of an N-terminal kinase catalytic domain and a C-terminal “caspase-recruitment” domain that interacts with caspase-like domains, indicating that RICK plays a role in the recruitment of caspase-8. This interpretation is supported by the fact that the expression of RICK in human 293T cells promotes activation of caspase-8 and potentiates the induction of apoptosis by various proteins involved in the CD95 apoptosis pathway (Inohara et al., supra).

Mitochondrial Protein Kinases

A novel class of eukaryotic kinases, related by sequence to prokaryotic histidine protein kinases, are the mitochondrial protein kinases (MPKs) which seem to have no sequence similarity with other eukaryotic protein kinases. These protein kinases are located exclusively in the mitochondrial matrix space and may have evolved from genes originally present in respiration-dependent bacteria which were endocytosed by primitive eukaryotic cells. MPKs are responsible for phosphorylation and inactivation of the branched-chain alpha-ketoacid dehydrogenase and pyruvate dehydrogenase complexes (Harris, R. A. et al. (1995) Adv. Enzyme Regul. 34:147-162). Five MPKs have been identified. Four members correspond to pyruvate dehydrogenase kinase isozymes, regulating the activity of the pyruvate dehydrogenase complex, which is an important regulatory enzyme at the interface between glycolysis and the citric acid cycle. The fifth member corresponds to a branched-chain alpha-ketoacid dehydrogenase kinase, important in the regulation of the pathway for the disposal of branched-chain amino acids. (Harris, R. A. et al. (1997) Adv. Enzyme Regul. 37:271-293). Both starvation and the diabetic state are known to result in a great increase in the activity of the pyruvate dehydrogenase kinase in the liver, heart and muscle of the rat. This increase contributes in both disease states to the phosphorylation and inactivation of the pyruvate dehydrogenase complex and conservation of pyruvate and lactate for gluconeogenesis (Harris (1995) supra).

Kinases with Non-Protein Substrates

Lipid and Inositol Kinases

Lipid kinases phosphorylate hydroxyl residues on lipid head groups. A family of kinases involved in phosphorylation of phosphatidylinositol (PI) has been described, each member phosphorylating a specific carbon on the inositol ring (Leevers, S. J. et al. (1999) Curr. Opin. Cell. Biol. 11:219-225). The phosphorylation of phosphatidylinositol is involved in activation of the protein kinase C signing pathway. The inositol phospholipids (phosphoinositides) intracellular signaling pathway begins with binding of a signaling molecule to a G-protein linked receptor in the plasma membrane. This leads to the phosphorylation of phosphatidylinositol (PI) residues on the inner side of the plasma membrane by inositol kinases, thus converting PI residues to the biphosphate state (PIP₂). PIP₂ is then cleaved into inositol triphosphate (IP₃) and diacylglycerol. These two products act as mediators for separate signaling pathways. Cellular responses that are mediated by these pathways are glycogen breakdown in the liver in response to vasopressin, smooth muscle contraction in response to acetylcholine, and thrombin-induced platelet aggregation.

PI 3-kinase (PI3K), which phosphorylates the D3 position of PI and its derivatives, has a central role in growth factor signal cascades involved in cell growth, differentiation, and metabolism. PI3K is a heterodimer consisting of an adapter subunit and a catalytic subunit. The adapter subunit acts as a scaffolding protein, interacting with specific tyrosine-phosphorylated proteins, lipid moieties, and other cytosolic factors. When the adapter subunit binds tyrosine phosphorylated targets, such as the insulin responsive substrate (IRS)-1, the catalytic subunit is activated and converts PI (4,5) bisphosphate (PIP₂) to PI (3,4,5) P₃ (PIP₃). PIP₃ then activates a number of other proteins, including PKA, protein kinase B (PKB), protein kinase C (PKC), glycogen synthase kinase (GSK)-3, and p70 ribosomal s6 kinase. PI3K also interacts directly with the cytoskeletal organizing proteins, Rac, rho, and cdc42 (Shepherd, P. R. et al. (1998) Biochem J. 333:471-490). Animal models for diabetes, such as obese and fat mice, have altered PI3K adapter subunit levels. Specific mutations in the adapter subunit have also been found in an insulin-resistant Danish population, suggesting a role for PI3K in type-2 diabetes (Shepard, supra).

An example of lipid kinase phosphorylation activity is the phosphorylation of D-erytbro-sphingosine to the sphingolipid metabolite, sphingosine-l-phosphate (SPP). SPP has emerged as a novel lipid second-messenger with both extracellular and intracellular actions (Kohama, T. et al. (1998) J. Biol. Chem. 273:23722-23728). Extracellularly, SPP is a ligand for the G-protein coupled receptor EDG-1 (endothelial-derived, G-protein coupled receptor). Intracellularly, SPP regulates cell growth, survival, motility, and cytoskeletal changes. SPP levels are regulated by sphingosine kinases that specifically phosphorylate D-erythro-sphingosine to SPP. The importance of sphingosine kinase in cell signaling is indicated by the fact that various stimuli, including platelet-derived growth factor (PDGF), nerve growth factor, and activation of protein kinase C, increase cellular levels of SPP by activation of sphingosine kinase, and the fact that competitive inhibitors of the enzyme selectively inhibit cell proliferation induced by PDGF (Kohama et al., supra).

Purine Nucleotide Kinases

The purine nucleotide kinases, adenylate kinase (ATP:AMP phosphotransferase, or AdK) and guanylate kinase (ATP:GMP phosphotransferase, or GuK) play a key role in nucleotide metabolism and are crucial to the synthesis and regulation of cellular levels of ATP and GTP, respectively. These two molecules are precursors in DNA and RNA synthesis in growing cells and provide the primary source of biochemical energy in cells (ATP), and signal transduction pathways (GTP). Inhibition of various steps in the synthesis of these two molecules has been the basis of many antiproliferative drugs for cancer and antiviral therapy (Pillwein, K et al. (1990) Cancer Res. 50:1576-1579).

AdK is found in almost all cell types and is especially abundant in cells having high rates of ATP synthesis and utilization such as skeletal muscle. In these cells AdK is physically associated with mitochondria and myofibrils, the subcellular structures that are involved in energy production and utilization, respectively. Recent studies have demonstrated a major function for AdK in transferring high energy phosphoryls from metabolic processes generating ATP to cellular components consuming ATP (Zeleznikar, R. J. et al. (1995) J. Biol. Chem 270:7311-7319). Thus AdK may have a pivotal role in maintaining energy production in cells, particularly those having a high rate of growth or metabolism such as cancer cells, and may provide a target for suppression of its activity to treat certain cancers. Alternatively, reduced AdK activity may be a source of various metabolic, muscle-energy disorders that can result in cardiac or respiratory failure and may be treatable by increasing AdK activity.

GuK, in addition to providing a key step in the synthesis of GTP for RNA and DNA synthesis, also fulfills an essential function in signal transduction pathways of cells through the regulation of GDP and GTP. Specifically, GTP binding to membrane associated G proteins mediates the activation of cell receptors, subsequent intracellular activation of adenyl cyclase, and production of the second messenger, cyclic AMP. GDP binding to G proteins inhibits these processes. GDP and GTP levels also control the activity of certain oncogenic proteins such as p21^{ras} known to be involved in control of cell proliferation and oncogenesis (Bos, J. L. (1989) Cancer Res. 49:4682-4689). High ratios of GTP:GDP caused by suppression of GuK cause activation of p21^{ras} and promote oncogenesis. Increasing GuK activity to increase levels of GDP and reduce the GTP:GDP ratio may provide a therapeutic strategy to reverse oncogenesis.

GuK is an important enzyme in the phosphorylation and activation of certain antiviral drugs useful in the treatment of herpes virus infections. These drugs include the guanine homologs acyclovir and buciclovir (Miller, W. H. and R. L. Miller (1980) J. Biol. Chem. 255:72047207; Stenberg, K. et al. (1986) J. Biol. Chem. 261:2134-2139). Increasing GuK activity in infected cells may provide a therapeutic strategy for augmenting the effectiveness of these drugs and possibly for reducing the necessary dosages of the drugs.

Pyrimidine Kinases The pyrimidine kinases are deoxycytidine kinase and thymidine kinase 1 and 2. Deoxycytidine kinase is located in the nucleus, and thymidine kinase 1 and 2 are found in the cytosol (Johansson, M. et al. (1997) Proc. Natl. Acad. Sci. USA 94:11941-11945). Phosphorylation of deoxyribonucleosides by pyrimidine kinases provides an alternative pathway for de novo synthesis of DNA precursors. The role of pyrimidine kinases, like purine kinases, in phosphorylation is critical to the activation of several chemotherapeutically important nucleoside analogues (Arner E. S. and S. Eriksson (1995) Pharmacol. Ther. 67:155-186).

The discovery of new human kinases and the polynucleotides encoding them satisfies a need in the art by providing new compositions which are useful in the diagnosis, prevention, and treatment of cancer, immune disorders, disorders affecting growth and development, cardiovascular diseases, and lipid disorders, and in the assessment of the effects of exogenous compounds on the expression of nucleic acid and amino acid sequences of human kinases.

### SUMMARY OF THE INVENTION

The invention features purified polypeptides, human kinases, referred to collectively as “PKIN” and individually as “PKIN-1,” “PKIN-2,” “PKIN-3,” “PKIN-4,” “PKIN-5,” “PKIN-6,” “PKIN-7,” “PKIN-8,” “PKIN-9,” “PKIN-10,” “PKIN-11,” “PKIN-12,” “PKIN-13,” “PKIN-14,” “PKIN-15,” “PKIN-16,” “PKIN-17,” “PKIN-18,” “PKIN-19,” “PKIN-20,” “PKIN-21,” “PKIN-22,” “PKIN-23,” “PKIN-24,” “PKIN-25,” and “PKIN-26.” In one aspect, the invention provides an isolated polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26. In one alternative, the invention provides an isolated polypeptide comprising the amino acid sequence of SEQ ID NO: 1-26.

The invention further provides an isolated polynucleotide encoding a polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26. In one alternative, the polynucleotide encodes a polypeptide selected from the group consisting of SEQ ID NO: 1-26. In another alternative, the polynucleotide is selected from the group consisting of SEQ ID NO: 27-52.

Additionally, the invention provides a recombinant polynucleotide comprising a promoter sequence operably linked to a polynucleotide encoding a polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26. In one alternative, the invention provides a cell transformed with the recombinant polynucleotide. In another alternative, the invention provides a transgenic organism comprising the recombinant polynucleotide.

The invention also provides a method for producing a polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26.

The invention further provides an isolated polynucleotide selected from the group consisting of a) a polynucleotide comprising a polynucleotide sequence selected from the group consisting of SEQ ID NO: 27-52, b) a polynucleotide comprising a naturally occurring polynucleotide sequence at least 90% identical to a polynucleotide sequence selected from the group consisting of SEQ ID NO: 27-52, c) a polynucleotide complementary to the polynucleotide of a), d) a polynucleotide complementary to the polynucleotide of b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO: 27-52, b) a polynucleotide comprising a naturally occurring polynucleotide sequence at least 90% identical to a polynucleotide sequence selected from the group consisting of SEQ ID NO: 27-52, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO: 27-52, b) a polynucleotide comprising a naturally occurring polynucleotide sequence at least 90% identical to a polynucleotide sequence selected from the group consisting of SEQ ID NO: 27-52, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention further provides a composition comprising an effective amount of a polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26, and a pharmaceutically acceptable excipient In one embodiment, the composition comprises an amino acid sequence selected from the group consisting of SEQ ID NO: 1-26. The invention additionally provides a method of treating a disease or condition associated with decreased expression of functional PKIN, comprising administering to a patient in need of such treatment the composition.

The invention also provides a method for screening a compound for effectiveness as an agonist of a polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional PKIN, comprising administering to a patient in need of such treatment the composition.

Additionally, the invention provides a method for screening a compound for effectiveness as an antagonist of a polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient In another alternative, the invention provides a method of treating a disease or condition associated with overexpression of functional PKIN, comprising administering to a patient in need of such treatment the composition.

The invention further provides a method of screening for a compound that specifically binds to a polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide selected from the group consisting of a) a polypeptide comprising an amino acid sequence selected from the group consisting of SEQ ID NO: 1-26, b) a polypeptide comprising a naturally occurring amino acid sequence at least 90% identical to an amino acid sequence selected from the group consisting of SEQ ID NO: 1-26, c) a biologically active fragment of a polypeptide having an amino acid sequence selected from the group consisting of SEQ ID NO: 1-26, and d) an immunogenic fragment of a polypeptide having an amino acid sequence selected from the group consisting of SEQ ID NO: 1-26. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a sequence selected from the group consisting of SEQ ID NO: 27-52, the method comprising a) exposing a sample comprising the target polynucleotide to a compound, and b) detecting altered expression of the target polynucleotide.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO: 27-52, ii) a polynucleotide comprising a naturally occurring polynucleotide sequence at least 90% identical to a polynucleotide sequence selected from the group consisting of SEQ ID NO: 27-52, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO: 27-52, ii) a polynucleotide comprising a naturally occurring polynucleotide sequence at least 90% identical to a polynucleotide sequence selected from the group consisting of SEQ ID NO: 27-52, iii) a polynucleotide complementary to the polynucleotide of i), iv) a polynucleotide complementary to the polynucleotide of ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for the full length polynucleotide and polypeptide sequences of the present invention.

Table 2 shows the GenBank identification number and annotation of the nearest GenBank homolog for polypeptides of the invention. The probability score for the match between each polypeptide and its GenBank homolog is also shown.

Table 3 shows structural features of polypeptide sequences of the invention, including predicted motifs and domains, along with the methods, algorithms, and searchable databases used for analysis of the polypeptides.

Table 4 lists the cDNA and/or genomic DNA fragments which were used to assemble polynucleotide sequences of the invention, along with selected fragments of the polynucleotide sequences.

Table 5 shows the representative cDNA library for polynucleotides of the invention.

Table 6 provides an appendix which describes the tissues and vectors used for construction of the cDNA libraries shown in Table 5.

Table 7 shows the tools, programs, and algorithms used to analyze the polynucleotides and polypeptides of the invention, along with applicable descriptions, references, and threshold parameters.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“PKIN” refers to the amino acid sequences of substantially purified PKIN obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of PKIN. Agonists may include proteins, nucleic acids, carbohydrates, small molecules, or any other compound or composition which modulates the activity of PKIN either by directly interacting with PKIN or by acting on components of the biological pathway in which PKIN participates.

An “allelic variant” is an alternative form of the gene encoding PKIN. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding PKIN include those sequences with deletions, insertions, or substitutions of different nucleotides, resulting in a polypeptide the same as PKIN or a polypeptide with at least one functional characteristic of PKIN. Included within this definition are polymorphisms which may or may not be readily detectable using a particular oligonucleotide probe of the polynucleotide encoding PKIN, and improper or unexpected hybridization to allelic variants, with a locus other than the normal chromosomal locus for the polynucleotide sequence encoding PKIN. The encoded protein may also be “altered,” and may contain deletions, insertions, or substitutions of amino acid residues which produce a silent change and result in a functionally equivalent PKIN. Deliberate amino acid substitutions may be made on the basis of similarity in polarity, charge, solubility, hydrophobicity, hydrophilicity, and/or the amphipathic nature of the residues, as long as the biological or immunological activity of PKIN is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of PKIN. Antagonists may include proteins such as antibodies, nucleic acids, carbohydrates, small molecules, or any other compound or composition which modulates the activity of PKIN either by directly interacting with PKIN or by acting on components of the biological pathway in which PKIN participates.

The term “antibody” refers to intact immunoglobulin molecules as well as to fragments thereof, such as Fab, F(ab′)₂, and Fv fragments, which are capable of binding an epitopic determinant. Antibodies that bind PKIN polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “antisense” refers to any composition capable of base-pairing with the “sense” (coding) strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic PKIN, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding PKIN or fragments of PKIN may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys,Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

A “fragment” is a unique portion of PKIN or the polynucleotide encoding PKIN which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of apolypeptide as shown in a certain defined sequence. Clearly these lengths are exemplary, and any length that is supported by the specification, including the Sequence Listing, tables, and figures, may be encompassed by the present embodiments.

A fragment of SEQ ID NO: 27-52 comprises a region of unique polynucleotide sequence that specifically identifies SEQ ID NO: 27-52, for example, as distinct from any other sequence in the genome from which the fragment was obtained. A fragment of SEQ ID NO: 27-52 is useful, for example, in hybridization and amplification technologies and in analogous methods that distinguish SEQ ID NO: 27-52 from related polynucleotide sequences. The precise length of a fragment of SEQ ID NO: 27-52 and the region of SEQ ID NO: 27-52 to which the fragment corresponds are routinely determinable by one of ordinary skill in the art based on the intended purpose for the fragment.

A fragment of SEQ ID NO: 1-26 is encoded by a fragment of SEQ ID NO: 27-52. A fragment of SEQ ID NO: 1-26 comprises a region of unique amino acid sequence that specifically identifies SEQ ID NO: 1-26. For example, a fragment of SEQ ID NO: 1-26 is useful as an immunogenic peptide for the development of antibodies that specifically recognize SEQ ID NO: 1-26. The precise length of a fragment of SEQ ID NO: 1-26 and the region of SEQ ID NO: 1-26 to which the fragment corresponds are routinely determinable by one of ordinary skill in the art based on the intended purpose for the fragment.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA

Generally, stringency of hybridization is expressed, in part, with reference to the temperature under which the wash step is carried out. Such wash temperatures are typically selected to be about 5° C to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of PKIN which is capable of eliciting an immune response when introduced into a living organism, for example, a mammal. The term “immunogenic fragment” also includes any polypeptide or oligopeptide fragment of PKIN which is useful in any of the antibody production methods disclosed herein or known in the art.

The term “microarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of PKIN. For example, modulation may cause an increase or a decrease in protein activity, binding characteristics, or any other biological, functional, or immunological properties of PKIN.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-lice or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an PKIN may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of PKIN.

“Probe” refers to nucleic acid sequences encoding PKIN, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} e, vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing PKIN, nucleic acids encoding PKIN, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternative splicing of exons during mRNA processing. The corresponding polypeptide may possess additional finctional domains or lack domains that are present in the reference molecule. Species variants are polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

The Invention

The invention is based on the discovery of new human human kinases (PKIN), the polynucleotides encoding PKIN, and the use of these compositions for the diagnosis, treatment, or prevention of cancer, immune disorders, disorders affecting growth and development, cardiovascular diseases, and lipid disorders.

Table 1 summarizes the nomenclature for the full length polynucleotide and polypeptide sequences of the invention. Each polynucleotide and its corresponding polypeptide are correlated to a single Incyte project identification number (Ilncyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown.

Table 2 shows sequences with homology to the polypeptides of the invention as identified by BLAST analysis against the GenBank protein (genpept) database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (Genbank ID NO:) of the nearest GenBank homolog. Column 4 shows the probability score for the match between each polypeptide and its GenBank homolog. Column 5 shows the annotation of the GenBank homolog along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are human kinases. For example, SEQ ID NO: 4 is 94% identical to rat serinelthreonine kinase (GenBank ID g2052189) as determined by the Basic Local Alignment Search Tool (BLAST). (See Table 2.) The BLAST probability score is 0.0, which indicates the probability of obtaining the observed polypeptide sequence alignment by chance. SEQ ID NO: 4 also contains a protein kinase domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO: 4 is a protein kinase. In an alternate example, SEQ ID NO: 23 is 88% identical to murine protein kinase (GenBank ID g406058) as determined by the Basic Local Alignment Search Tool (BLAST). (See Table 2.) The BLAST probability score is 0.0, which indicates the probability of obtaining the observed polypeptide sequence alignment by chance. SEQ ID NO: 23 also contains an eukaryotic protein kinase domain as determined by searching for statistically significant matches in the bidden Markov model (HMM)-based PFAM database of conserved protein family domains. (See Table 3.) Data from BLIMPS, MOTIFS, and PROFILESCAN analyses provide further corroborative evidence that SEQ ID NO: 23 is a protein kinase. In an alternate example, SEQ ID NO: 6 is 85% identical to rabbit myosin light chain kinase (GenBank ID g165506) as determined by the Basic Local Alignment Search Tool (BLAST). (See Table 2.) The BLAST probability score is 1.5e-272, which indicates the probability of obtaining the observed polypeptide sequence alignment by chance. SEQ ID NO: 6 also contains a eukaryotic protein kinase domain as determined by searching for statistically significant matches in the hidden Markov model (HMM)-based PFAM database of conserved protein family domains. (See Table 3.) Data from BLIMPS and MOTIFS analyses provide further corroborative evidence that SEQ ID NO: 6 is a myosin light chain kinase. In an alternate example, SEQ ID NO: 1 is 64% identical to murine serinelthreonine kinase (GenBank ID g404634) as determined by the Basic Local Alignment Search Tool (BLAST). (See Table 2.) The BLAST probability score is 4.5e-60, which indicates the probability of obtaining the observed polypeptide sequence alignment by chance. SEQ ID NO: 1 also contains a protein kinase domain as determined by searching for statistically significant matches in the hidden Markov model (HMM)-based PFAM database of conserved protein family domains. (See Table 3.) Data from MOTIFS, BLIMPS and PROFILESCAN analyses provide further corroborative evidence that SEQ ID NO: 1 is a protein kinase, notably a serine/threonine kinase. In an alternate example, SEQ ID NO: 19 is 49% identical to human G-protein-coupled receptor kinase GRK4-beta (GenBank ID g992672) as determined by the Basic Local Alignment Search Tool (BLAST). (See Table 2.) The BLAST probability score is 4.3e-129, which indicates the probability of obtaining the observed polypeptide sequence alignment by chance. SEQ ID NO: 19 also contains a regulator of G-protein signaling domain as determined by searching for statistically significant matches in the hidden Markov model (HMM-based PFAM database of conserved protein family domains. (See Table 3.) Data from BLIMPS, MOTIFS, and PROFILESCAN analyses provide further corroborative evidence that SEQ ID NO: 19 is a G-protein-coupled receptor kinase. SEQ ID NO: 2-3, SEQ ID NO: 5, SEQ ID NO: 7-18, SEQ ID NO: 20-22 and SEQ ID NO: 24-26 were analyzed and annotated in a similar manner. The algorithms and parameters for the analysis of SEQ ID NO: 1-26 are described in Table 7.

As shown in Table 4, the full length polynucleotide sequences of the present invention were assembled using cDNA sequences or coding (exon) sequences derived from genomic DNA, or any combination of these two types of sequences. Columns 1 and 2 list the polynucleotide sequence identification number (Polynucleotide SEQ ID NO:) and the corresponding Incyte polynucleotide consensus sequence number (Incyte Polynucleotide ID) for each polynucleotide of the invention. Column 3 shows the length of each polynucleotide sequence in basepairs. Column 4 lists fragments of the polynucleotide sequences which are useful, for example, in hybridization or amplification technologies that identify SEQ ID NO: 27-52 or that distinguish between SEQ ID NO: 27-52 and related polynucleotide sequences. Column 5 shows identification numbers corresponding to cDNA sequences, coding sequences (exons) predicted from genomic DNA, and/or sequence assemblages comprised of both cDNA and genomic DNA. These sequences were used to assemble the full length polynucleotide sequences of the invention. Columns 6 and 7 of Table 4 show the nucleotide start (5′) and stop (3′) positions of the cDNA and/or genomic sequences in column 5 relative to their respective full length sequences.

The identification numbers in Column 5 of Table 4 may refer specifically, for example, to Incyte cDNAs along with their corresponding cDNA libraries. For example, 6829315H1 is the identification number of an Incyte cDNA sequence, and SINTNOR01 is the cDNA library from which it is derived. Incyte cDNAs for which cDNA libraries are not indicated were derived from pooled cDNA libraries (e.g., 55057226H1). Alternatively, the identification numbers in column 5 may refer to GenBank cDNAs or ESTs (e.g., g2954208) which contributed to the assembly of the full length polynucleotide sequences. In addition, the identification numbers in column 5 may identify sequences derived from the ENSEMBL (The Sanger Centre, Cambridge, UK) database (i.e., those sequences including the designation “ENST”). Alternatively, the identification numbers in column 5 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the identification numbers in column 5 may refer to assemblages of both cDNA and Genscan-predicted exons brought together by an “exon stitching” algorithm. For example, FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the identification numbers in column 5 may refer to assemblages of exons brought together by an “exon-stretching” algorithm. For example, FLXXXXXX_gAAAAA_gBBBBB_{—}1_N is the identification number of a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, for |
| ENST | example, GENSCAN (Stanford University, CA, USA) |
| | or FGENES (Computer Genomics Group, The Sanger |
| | Centre, Cambridge, UK). |
| GBI | Hand-edited analysis of genomic sequences. |
| FL | Stitched or stretched genomic sequences (see Example V). |

In some cases, Incyte cDNA coverage redundant with the sequence coverage shown in column was obtained to confirm the final consensus polynucleotide sequence, but the relevant Incyte cDNA identification numbers are not shown.

Table 5 shows the representative cDNA libraries for those full length polynucleotide sequences which were assembled using Incyte cDNA sequences. The representative cDNA library is the Incyte cDNA library which is most frequently represented by the Incyte cDNA sequences which were used to assemble and confirm the above polynucleotide sequences. The tissues and vectors which were used to construct the cDNA libraries shown in Table 5 are described in Table 6.

The invention also encompasses PKIN variants. A preferred PKIN variant is one which has at least about 80%, or alternatively at least about 90%, or even at least about 95% amino acid sequence identity to the PKIN amino acid sequence, and which contains at least one functional or structural characteristic of PKIN.

The invention also encompasses polynucleotides which encode PKIN. In a particular embodiment, the invention encompasses a polynucleotide sequence comprising a sequence selected from the group consisting of SEQ ID NO: 27-52, which encodes PKIN. The polynucleotide sequences of SEQ ID NO: 27-52, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding PKIN. In particular, such a variant polynucleotide sequence will have at least about 70%, or alternatively at least about 85%, or even at least about 95% polynucleotide sequence identity to the polynucleotide sequence encoding PKIN. A particular aspect of the invention encompasses a variant of a polynucleotide sequence comprising a sequence selected from the group consisting of SEQ ID NO: 27-52 which has at least about 70%, or alternatively at least about 85%, or even at least about 95% polynucleotide sequence identity to a nucleic acid sequence selected from the group consisting of SEQ ID NO: 27-52. Any one of the polynucleotide variants described above can encode an amino acid sequence which contains at least one functional or structural characteristic of PKIN.

It will be appreciated by those skilled in the art that as a result of the degeneracy of the genetic code, a multitude of polynucleotide sequences encoding PKIN, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring PKIN, and all such variations are to be considered as being specifically disclosed.

Although nucleotide sequences which encode PKIN and its variants are generally capable of hybridizing to the nucleotide sequence of the naturally occurring PKIN under appropriately selected conditions of stringency, it may be advantageous to produce nucleotide sequences encoding PKIN or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding PKIN and its derivatives without altering the encoded amino acid sequences include the production of RNA transcripts having more desirable properties, such as a greater half-life, than transcripts produced from the naturally occurring sequence.

The invention also encompasses production of DNA sequences which encode PKIN and PKIN derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding PKIN or any fragment thereof.

Also encompassed by the invention are polynucleotide sequences that are capable of hybridizing to the claimed polynucleotide sequences, and, in particular, to those shown in SEQ ID NO: 27-52 and fragments thereof under various conditions of stringency. (See, e.g., Wahl, G. M. and S. L. Berger (1987) Methods Enzymol. 152:399407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosysterns). Sequencing is then carried out using either the ABI 373 or 377 DNA sequencing system (Applied Biosystems), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding PKIN may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode PKIN may be cloned in recombinant DNA molecules that direct expression of PKIN, or fragments or functional equivalents thereof, in appropriate host cells. Due to the inherent degeneracy of the genetic code, other DNA sequences which encode substantially the same or a functionally equivalent amino acid sequence may be produced and used to express PKIN.

The nucleotide sequences of the present invention can be engineered using methods generally known in the art in order to alter PKIN-encoding sequences for a variety of purposes including, but not limited to, modification of the cloning, processing, and/or expression of the gene product DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C. -C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of PKIN, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding PKIN may be synthesized, in whole or in part, using chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, PKIN itself or a fragment thereof may be synthesized using chemical methods. For example, peptide synthesis can be performed using various solution-phase or solid-phase techniques. (See, e.g., Creighton, T. (1984) Proteins, Structures and Molecular Properties, W H Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of PKIN, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392421.) The composition of the synthetic peptides may be confirmed by amino acid analysis or by sequencing. (See, e.g., Creighton, supra, pp. 28-53.)

In order to express a biologically active PKIN, the nucleotide sequences encoding PKIN or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding PKIN. Such elements may vary in their strength and specificity. Specific initiation signals may also be used to achieve more efficient translation of sequences encoding PKIN. Such signals include the ATG initiation codon and adjacent sequences, e.g. the Kozak sequence. In cases where sequences encoding PEKIN and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing sequences encoding PKIN and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express sequences encoding PKIN. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotide sequences encoding PKIN. For example, routine cloning, subcloning, and propagation of polynucleotide sequences encoding PKIN can be achieved using a multifunctional E. coli vector such as PBLUESCRIPT (Stratagene, La Jolla Calif.) or PSPORT1 plasmid (Life Technologies). Ligation of sequences encoding PKIN into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S.M. Schuster (1989) J. Biol. Chem. 264:5503-5509.) When large quantities of PKIN are needed, e.g. for the production of antibodies, vectors which direct high level expression of PKIN may be used. For example, vectors containing the strong, inducible SP6 or T7 bacteriophage promoter may be used.

Yeast expression systems may be used for production of PKIN. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of PKIN. Transcription of sequences encoding PKIN may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding PKIN may be ligated into an adenovirus transcription/translation complex consisting of the late promoter and tripartite leader sequence. Insertion in a non-essential E1 or E3 region of the viral genome may be used to obtain infective virus which expresses PKIN in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.) For long term production of recombinant proteins in mammalian systems, stable expression of PKIN in cell lines is preferred For example, sequences encoding PKIN can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphonbosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding PKIN is inserted within a marker gene sequence, transformed cells containing sequences encoding PKIN can be identified by the absence of marker gene function. Alternatively, a marker gene can be placed in tandem with a sequence encoding PKIN under the control of a single promoter. Expression of the marker gene in response to induction or selection usually indicates expression of the tandem gene as well.

In general, host cells that contain the nucleic acid sequence encoding PKIN and that express. PKIN may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of PKIN using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on PKIN is preferred, but a competitive binding assay may be employed. These and other assays are well known in the art. (See, e.g., Hampton, R. et al. (1990) Serological Methods, a Laboratory Manual, APS Press, St. Paul Minn., Sect IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding PKIN include oligolabeling, nick translation, end-labeling, or PCR amplification using a labeled nucleotide. Alternatively, the sequences encoding PKIN, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding PKIN may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode PKIN may be designed to contain signal sequences which direct secretion of PKIN through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant nucleic acid sequences encoding PKIN may be ligated to a heterologous sequence resulting in translation of a fusion protein in any of the aforementioned host systems. For example, a chimeric PKIN protein containing a heterologous moiety that can be recognized by a commercially available antibody may facilitate the screening of peptide libraries for inhibitors of PKIN activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the PKIN encoding sequence and the heterologous protein sequence, so that PKIN may be cleaved away from the heterologous moiety following purification. Methods for fusion protein expression and purification are discussed in Ausubel (1995, supra, ch 10). A variety of commercially available kits may also be used to facilitate expression and purification of fusion proteins.

In a further embodiment of the invention, synthesis of radiolabeled PKIN may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

PKIN of the present invention or fragments thereof may be used to screen for compounds that specifically bind to PKIN. At least one and up to a plurality of test compounds may be screened for specific binding to PKIN. Examples of test compounds include antibodies, oligonucleotides, proteins (e.g., receptors), or small molecules.

In one embodiment, the compound thus identified is closely related to the natural ligand of PKIN, e.g., a ligand or fragment thereof, a natural substrate, a structural or functional mimetic, or a natural binding partner. (See, e.g., Coligan, J. E. et al. (1991) Current Protocols in Immunology 1(2): Chapter 5.) Similarly, the compound can be closely related to the natural receptor to which PKIN binds, or to at least a fragment of the receptor, e.g., the ligand binding site. In either case, the compound can be rationally designed using known techniques. In one embodiment, screening for these compounds involves producing appropriate cells which express PKIN, either as a secreted protein or on the cell membrane. Preferred cells include cells from mammals, yeast, Drosophila, or E. coli. Cells expressing PKIN or cell membrane fractions which contain PKIN are then contacted with a test compound and binding, stimulation, or inhibition of activity of either PKIN or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with PKIN, either in solution or affixed to a solid support, and detecting the binding of PKIN to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

PKIN of the present invention or fragments thereof may be used to screen for compounds that modulate the activity of PKIN. Such compounds may include agonists, antagonists, or partial or inverse agonists. In one embodiment, an assay is performed under conditions permissive for PKIN activity, wherein PKIN is combined with at least one test compound, and the activity of PKIN in the presence of a test compound is compared with the activity of PKIN in the absence of the test compound A change in the activity of PKIN in the presence of the test compound is indicative of a compound that modulates the activity of PKIN. Alternatively, a test compound is combined with an in vitro or cell-free system comprising PKIN under conditions suitable for PKIN activity, and the assay is performed. In either of these assays, a test compound which modulates the activity of PKIN may do so indirectly and need not come in direct contact with the test compound. At least one and up to a plurality of test compounds may be screened.

In another embodiment, polynucleotides encoding PKIN or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-1oxP system to knockout a gene of interest in a tissue- or developmental stage-specific manner (Marth, J. D. (1996) Ci Invest 97:1999-2002; Wagner, K. U. et al. (1997) Nucleic Acids Res. 25:43234330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding PKIN may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding PKIN can also be used to create “knockin” humanized animals (pigs) or transgenic animals (mice or rats) to model human disease. With knockin technology, a region of a polynucleotide encoding PKIN is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress PKIN, e.g., by secreting PKIN in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of PKIN and human kinases. In addition, the expression of PKIN is closely associated with lipid disorders, pancreatic islet cells, liver disease, leukocytes, umbilical endothelial cells, cancer, as well as, normal and diseased brain, renal, reproductive, bladder tumor, posterior hippocampus, kidney, small intestine, colon, and digestive tissues. Therefore, PKIN appears to play a role in cancer, immune disorders, disorders affecting growth and development, cardiovascular diseases, and lipid disorders. In the treatment of disorders associated with increased PKIN expression or activity, it is desirable to decrease the expression or activity of PKIN. In the treatment of disorders associated with decreased PKIN expression or activity, it is desirable to increase the expression or activity of PKIN.

Therefore, in one embodiment, PKIN or a fragment or derivative thereof may be administered to a subject to treat or prevent a disorder associated with decreased expression or activity of PKIN. Examples of such disorders include, but are not limited to, a cancer, such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus, leukemias such as multiple myeloma and lymphomas such as Hodgkin's disease; an immune disorder, such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjógren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a growth and developmental disorder, such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus, renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a cardiovascular disease, such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; and a lipid disorder, such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, lipid myopathies, and obesity.

In another embodiment, a vector capable of expressing PKIN or a fragment or derivative thereof may be administered to a subject to treat or prevent a disorder associated with decreased expression or activity of PKIN including, but not limited to, those described above.

In a further embodiment, a composition comprising a substantially purified PKIN in conjunction with a suitable pharmaceutical carrier may be administered to a subject to treat or prevent a disorder associated with decreased expression or activity of PKIN including, but not limited to, those provided above.

In still another embodiment, an agonist which modulates the activity of PKIN may be administered to a subject to treat or prevent a disorder associated with decreased expression or activity of PKIN including, but not limited to, those listed above.

In a further embodiment, an antagonist of PKIN may be administered to a subject to treat or prevent a disorder associated with increased expression or activity of PKIN. Examples of such disorders include, but are not limited to, those cancer, immune disorders, disorders affecting growth and development, cardiovascular diseases, and lipid disorders described above. In one aspect, an antibody which specifically binds PKIN may be used directly as an antagonist or indirectly as a targeting or delivery mechanism for bringing a pharmaceutical agent to cells or tissues which express PKIN.

In an additional embodiment, a vector expressing the complement of the polynucleotide encoding PKIN may be administered to a subject to treat or prevent a disorder associated with increased expression or activity of PKIN including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of PKIN may be produced using methods which are generally known in the art. In particular, purified PKIN may be used to produce antibodies or to screen libraries of pharmaceutical agents to identify those which specifically bind PKIN. Antibodies to PKIN may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use.

For the production of antibodies, various hosts including goats, rabbits, rats, mice, humans, and others may be immunized by injection with PKIN or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to PKIN have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of PKIN amino acids may be fused with those of another protein, such as KLH, and antibodies to the chimeric molecule may be produced.

Monoclonal antibodies to PKIN may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495497; Kozbor, D. et al. (1985) J. Immunol. Methods 81:3142; Cote, R. J. et al. (1983) Proc. Natl. Acad. Sci. USA 80;2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452454.) Alternatively, techniques described for the production of single chain antibodies may be adapted, using methods known in the art, to produce PKIN-specific single chain antibodies. Antibodies with related specificity, but of distinct idiotypic composition, may be generated by chain shuffling from random combinatorial inmunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for PKIN may also be generated. For example, such fragments include, but are not limited to, F(ab)₂ fragments produced by pepsin digestion of the antibody molecule and Fab fragments generated by reducing the disulfide bridges of the F(ab)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between PKIN and its specific antibody. A two-site, monoclonal-based immunoassay utilizing monoclonal antibodies reactive to two non-interfering PKIN epitopes is generally used, but a competitive binding assay may also be employed (Pound, supra).

Various methods such as Scatchard analysis in conjunction with radioinmmunoassay techniques may be used to assess the affinity of antibodies for PKIN. Affinity is expressed as an association constant, Kₐ, which is defined as the molar concentration of PKIN-antibody complex divided by the molar concentrations of free antigen and free antibody under equilibrium conditions. The Kₐ determined for a preparation of polyclonal antibodies, which are heterogeneous in their affinities for multiple PKIN epitopes, represents the average affinity, or avidity, of the antibodies for PKIN. The Kₐ determined for a preparation of monoclonal antibodies, which are monospecific for a particular PKIN epitope, represents a true measure of affinity. High-affinity antibody preparations with Kₐ ranging from about 10⁹ to 10¹² L/mole are preferred for use in immunoassays in which the PKIN-antibody complex must withstand rigorous manipulations. Low-affinity antibody preparations with Kₐ ranging from about 10⁶ to 10⁷ L/mole are preferred for use in immunopurification and similar procedures which ultimately require dissociation of PKIN, preferably in active form, from the antibody (Catty, D. (1988) Antibodies, Volume I: A Practical Approach, IRL Press, Washington DC; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of PKIN-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding PKIN, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding PKIN. Such technology is well known in the art, and antisense oligonucleotides or larger fragments can be designed from various locations along the coding or control regions of sequences encoding PKIN. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Cli. Immunol. 102(3):469475; and Scanlon, K. J. et al. (1995) 9(13): 1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artficial viral envelopes, and other systems known in the art. (See, e.g., Rossi, J. J. (1995) Br. Med. Bull. 51(l):217-225; Boado, R. J. et al. (1998) J. Pharm. Sci. 87(11):1308-1315; and Moris, M. C. et al. (1997) Nucleic Acids Res. 25(14):2730-2736.)

In another embodiment of the invention, polynucleotides encoding PKIN may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA. 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in PKIN expression or regulation causes disease, the expression of PKIN from an appropriate population of transduced cells may alleviate the clinical manifestations caused by the genetic deficiency.

In a further embodiment of the invention, diseases or disorders caused by deficiencies in PKIN are treated by constructing mammalian expression vectors encoding PKIN and introducing these vectors by mechanical means into PKIN-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J -L. and H. Récipon (1998) Curr. Opin. Biotechnol. 9:445450).

Expression vectors that may be effective for the expression of PKIN include, but are not limited to, the PCDNA 3.1, EPITAG, PRCCMV2, PREP, PVAX vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). PKIN may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and Blau, H. M. supra)), or (iii) a tissue-specific promoter or the native promoter of the endogenous gene encoding PKIN from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to PKIN expression are treated by constructing a retrovirus vector consisting of (i) the polynucleotide encoding PKIN under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSV_{g} (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:47074716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding PKIN to cells which have one or more genetic abnormalities with respect to the expression of PKIN. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another alternative, a herpes-based, gene therapy delivery system is used to deliver polynucleotides encoding PIN to target cells which have one or more genetic abnormalities with respect to the expression of PKIN. The use of herpes simplex virus (HSV)-based vectors may be especially valuable for introducing PKIN to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver polynucleotides encoding PKIN to target cells. The biology of the prototypic alphavirus, Semkliki Forest Virus (SFV), has been studied extensively and gene transfer vectors have been based on the SFV genome (Garoff, H. and K. -J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for PKIN into the alphavirus genome in place of the capsid-coding region results in the production of a large number of PKIN-coding RNAs and the synthesis of high levels of PKIN in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of PKIN into a variety of cell types. The specific transduction of a subset of cells in a population may require the sorting of cells prior to transduction The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions-10 and +10 from the start site, may also be employed to inhibit gene expression. Similarly, inbibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding PKIN.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding PKIN. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding PKIN. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased PKIN expression or activity, a compound which specifically inhibits expression of the polynucleotide encoding PKIN may be therapeutically useful, and in the treatment of disorders associated with decreased PKIN expression or activity, a compound which specifically promotes expression of the polynucleotide encoding PKIN may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding PKIN is exposed to at least one test compound thus obtained. The sample may comprise, for example, an intact or permeabilized cell, or an in vitro cell-free or reconstituted biochemical system. Alterations in the expression of a polynucleotide encoding PKIN are assayed by any method commonly known in the art. Typically, the expression of a specific nucleotide is detected by hybridization with a probe having a nucleotide sequence complementary to the sequence of the polynucleotide encoding PKIN. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No.5,686,242; Bruice, T. W. et al. (2000) U.S. Pat. No.6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of PKIN, antibodies to PKIN, and mimetics, agonists, antagonists, or inhibitors of PKIN.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising PKIN or fragments thereof. For example, liposome preparations containing a cell-impermeable macromolecule may promote cell fusion and intracellular delivery of the macromolecule. Alternatively, PKIN or a fragment thereof may be joined to a short cationic N-terminal portion from the HIV Tat-I protein Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example PKIN or fragments thereof, antibodies of PKIN, and agonists, antagonists or inhibitors of PKIN, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind PKIN may be used for the diagnosis of disorders characterized by expression of PKIN, or in assays to monitor patients being treated with PKIN or agonists, antagonists, or inhibitors of PKIN. Antibodies useful for diagnostic purposes may be prepared in the same manner as described above for therapeutics. Diagnostic assays for PKIN include methods which utilize the antibody and a label to detect PKIN in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring PKIN, including ELISAs, RIAs, and FACS, are known in the art and provide a basis for diagnosing altered or abnormal levels of PKIN expression. Normal or standard values for PKIN expression are established by combining body fluids or cell extracts taken from normal mammalian subjects, for example, human subjects, with antibodies to PKIN under conditions suitable for complex formation. The amount of standard complex formation may be quantitated by various methods, such as photometric means. Quantities of PKIN expressed in subject, control, and disease samples from biopsied tissues are compared with the standard values. Deviation between standard and subject values establishes the parameters for diagnosing disease.

In another embodiment of the invention, the polynucleotides encoding PKIN may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of PKIN may be correlated with disease. The diagnostic assay may be used to determine absence, presence, and excess expression of PKIN, and to monitor regulation of PKIN levels during therapeutic intervention.

In one aspect, hybridization with PCR probes which are capable of detecting polynucleotide sequences, including genomic sequences, encoding PKIN or closely related molecules may be used to identify nucleic acid sequences which encode PKIN. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding PKIN, allelic variants, or related sequences.

Probes may also be used for the detection of related sequences, and may have at least 50% sequence identity to any of the PKIN encoding sequences. The hybridization probes of the subject invention may be DNA or RNA and may be derived from the sequence of SEQ ID NO: 27-52 or from genomic sequences including promoters, enhancers, and introns of the PKIN gene.

Means for producing specific hybridization probes for DNAs encoding PKIN include the cloning of polynucleotide sequences encoding PKIN or PKIN derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding PKIN may be used for the diagnosis of disorders associated with expression of PKIN. Examples of such disorders include, but are not limited to, a cancer, such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus, leukemias such as multiple myeloma and lymphomas such as Hodgkin's disease; an immune disorder, such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoartritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a growth and developmental disorder, such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart kidney, liver, lung, muscle, ovary, pancreas, parathyroid, penis, prostate, salivary glands, skin, spleen, testis, thymus, thyroid, and uterus, renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a cardiovascular disease, such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; and a lipid disorder, such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertnglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, lipid myopathies, and obesity. The polynucleotide sequences encoding PKIN may be used in Southern or northern analysis, dot blot, or other membrane-based technologies; in PCR technologies; in dipstick, pin, and multiformat ELISA-like assays; and in microarrays utilizing fluids or tissues from patients to detect altered PKIN expression. Such qualitative or quantitative methods are well known in the art.

In a particular aspect, the nucleotide sequences encoding PKIN may be useful in assays that detect the presence of associated disorders, particularly those mentioned above. The nucleotide sequences encoding PKIN may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding PKIN in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of PKIN, a normal or standard profile for expression is established. This may be accomplished by combining body fluids or cell extracts taken from normal subjects, either animal or human, with a sequence, or a fragment thereof, encoding PKIN, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding PKIN may involve the use of PCR. These oligomers may be chemically synthesized, generated enzymatically, or produced in vitro. Oligomers will preferably contain a fragment of a polynucleotide encoding PKIN, or a fragment of a polynucleotide complementary to the polynucleotide encoding PKIN, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding PKIN may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from the polynucleotide sequences encoding PKIN are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

Methods which may also be used to quantify the expression of PKIN include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, PKIN, fragments of PKIN, or antibodies specific for PKIN may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http:/www.niehs.nih.gov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for PKIN to quantify the levels of PKIN expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention In another embodiment, the toxicity of a test compound is assessed by treating a biological sample containing proteins with the test compound Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London, hereby expressly incorporated by reference.

In another embodiment of the invention, nucleic acid sequences encoding PKIN may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet 7:149-154.) Once mapped, the nucleic acid sequences of the invention may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding PKIN on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, PKIN, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between PKIN and the agent being tested may be measured.

Another technique for drug screening provides for high throughput screening of compounds having suitable binding affinity to the protein of interest (See, e.g., Geysen, et al. (1984) PCT application WO84/03564.) In this method, large numbers of different small test compounds are synthesized on a solid substrate. The test compounds are reacted with PKIN, or fragments thereof, and washed. Bound PKIN is then detected by methods well known in the art. Purified PKIN can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding PKIN specifically compete with a test compound for binding PKIN. In this manner, antibodies can be used to detect the presence of any peptide which shares one or more antigenic determinants with PKIN.

In additional embodiments, the nucleotide sequences which encode PKIN may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent The following embodiments are, therefore, to be construed as merely illustrative, and not limitative of the remainder of the disclosure in any way whatsoever.

The disclosures of all patents, applications and publications, mentioned above and below, including U.S. Ser. No. 60/212,073, U.S. Ser. No. 60/213,467, U.S. Ser. No. 60/215,651, U.S. Ser. No. 60/216,605, U.S. Ser. No. 60/218,372, and U.S. Ser. No. 60/228,056 are expressly incorporated by reference herein.

### EXAMPLES

I. Construction of cDNA Libraries

Incyte cDNAs were derived from cDNA libraries described in the LIFESEQ GOLD database (Incyte Genomics, Palo Alto Calif.) and shown in Table 4, column 5. Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), PCDNA2.1 plasmid (Invitrogen, Carlsbad Calif.), PBK-CMV plasmid (Stratagene), or pINCY (Incyte Genomics, Palo Alto Calif.), or derivatives thereof. Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg MD); and QIAWELL 8 Plasmid, QIAWELL 8 Plus Plasmid, QIAWEILL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM, and hidden Markov model (HMM)-based protein family databases such as PFAM. (HMM is a probabilistic approach which analyzes consensus primary structures of gene families. See, for example, Eddy, S. R. (1996) Curr. Opin. Struct Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide of the invention may begin at any of the methionine residues of the full length translated polypeptide. Full length polypeptide sequences were subsequently analyzed by querying against databases such as the GenBank protein databases (genpept), SwissProt, BLOCKS, PRINTS, DOMO, PRODOM, Prosite, and hidden Markov model (HMM)-based protein family databases such as PFAM. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO: 27-52. Fragments from about 20 to about 4000 nucleotides which are useful in hybridization and amplification technologies are described in Table 4, column 4.

IV. Identification and Editing of Coding Sequences from Genomic DNA

Putative human kinases were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode human kinases, the encoded polypeptides were analyzed by querying against PFAM models for human kinases. Potential human kinases were also identified by homology to Incyte cDNA sequences that had been annotated as human kinases. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example m were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genornic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of PKIN Encoding Polynucleotides

The sequences which were used to assemble SEQ ID NO: 27-52 were compared with sequences from the Incyte LIFESEQ database and public domain databases using BLAST and other implementations of the Smith-Waterman algorithm. Sequences from these databases that matched SEQ ID NO: 27-52 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

In this manner, SEQ ID NO: 27 was mapped to chromosome 19 and SEQ ID NO: 35 was mapped to chromosome 15 within the interval from 72.30 to 77.30 centiMorgans. SEQ ID NO: 48 was mapped to chromosome 10 within the interval from 93.80 to 96.90 centiMorgans. SEQ ID NO: 49 was mapped to chromosome 13 within the interval from 11.60 to 22.80 centiMorgans, to chromosome 17 within the interval from 0.60 to 14.80 centiMorgans, and to chromosome 20 within the interval from 57.70 to 64.10 centiMorgans. More than one map location is reported for SEQ ID NO: 49, indicating that sequences having different map locations were assembled into a single cluster. This situation occurs, for example, when sequences having strong similarity, but not complete identity, are assembled into a single cluster.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotide sequences encoding PKIN are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding PKIN. cDNA sequences and cDNA library/tissue information are found in the LIFESEQ GOLD database (Incyte Genomics, Palo Alto Calif.).

VIII. Extension of PKIN Encoding Polynucleotides

Full length polynucleotide sequences were also produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 mnol of each primer, reaction buffer containing Mg²⁺, (NH₄)₂SO₄, and 2-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharnacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2x carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, full length polynucleotide sequences are verified using the above procedure or are used to obtain 5′ regulatory sequences using the above procedure along with oligonucleotides designed for such extension, and an appropriate genomic library.

IX. Labeling and Use of Individual Hybridization Probes

Hybridization probes derived from SEQ ID NO: 27-52 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham NH). Hybridization is carried out for 16 hours at 40° C. To remove nonspecific signals, blots are sequentially washed at room temperature under conditions of up to, for example, 0.1×saline sodium citrate and 0.5% sodium dodecyl sulfate. Hybridization patterns are visualized using autoradiography or an alternative imaging means and compared.

X. Microarrays

The linkage or synthesis of array elements upon a microarray can be achieved utilizing photolithography, piezoelectric printing (ink-jet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21 mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 μg/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second washbuffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20×microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

XI. Complementary Polynucleotides

Sequences complementary to the PKIN-encoding sequences, or any parts thereof, are used to detect, decrease, or inhibit expression of naturally occurring PKIN. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of PKIN. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the PKIN-encoding transcript.

XII. Expression of PKIN

Expression and purification of PKIN is achieved using bacterial or virus-based expression systems. For expression of PKIN in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express PKIN upon induction with isopropyl beta-D-thiogalactopyranoside (IPTG). Expression of PKIN in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding PKIN by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, PKIN is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from PKIN at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified PKIN obtained by these methods can be used directly in the assays shown in Examples XVI, XVII, and XVIII where applicable.

XIII. Functional Assays

PKIN function is assessed by expressing the sequences encoding PKIN at physiologically elevated levels in mammalian cell culture systems. cDNA is subcloned into a mammalian expression vector containing a strong promoter that drives high levels of cDNA expression. Vectors of choice include PCMV SPORT (Life Technologies) and PCR3.1 (Invitrogen, Carlsbad Calif.), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of PKIN on gene expression can be assessed using highly purified populations of cells transfected with sequences encoding PKIN and either CD64 or CD64GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding PKIN and other genes of interest can be analyzed by northern analysis or microarray techniques.

XIV. Production of PKIN Specific Antibodies

PKIN substantially purified using polyacrylamide gel electrophoresis (PAGE; see, e.g., Harrington, M. G. (1990) Methods Enzymol. 182:488-495), or other purification techniques, is used to immunize rabbits and to produce antibodies using standard protocols.

Alternatively, the PKIN amino acid sequence is analyzed using LASERGENE software DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-PKIN activity by, for example, binding the peptide or PKIN to a substrate, blocking with 1% BSA, reacting with rabbit antisera, washing, and reacting with radio-iodinated goat anti-rabbit IgG.

XV. Purification of Naturally Occurring PKIN Using Specific Antibodies

Naturally occurring or recombinant PKIN is substantially purified by immunoaffinity chromatography using antibodies specific for PKIN. An immunoaffinity column is constructed by covalently coupling anti-PKIN antibody to an activated chromatographic resin, such as CNBr-activated SEPHAROSE (Amersham Pharmacia Biotech). After the coupling, the resin is blocked and washed according to the manufacturer's instructions.

Media containing PKIN are passed over the immunoaffinity column, and the column is washed under conditions that allow the preferential absorbance of PKIN (e.g., high ionic strength buffers in the presence of detergent). The column is eluted under conditions that disrupt antibody/PKIN binding (e.g., a buffer of pH 2 to pH 3, or a high concentration of a chaotrope, such as urea or thiocyanate ion), and PKIN is collected.

XVI. Identification of Molecules which Interact with PKIN

PKIN, or biologically active fragments thereof, are labeled with ¹²⁵I Bolton-Hunter reagent. (See, e.g., Bolton A. E. and W. M. Hunter (1973) Biochem. J. 133:529-539.) Candidate molecules previously arrayed in the wells of a multi-well plate are incubated with the labeled PKIN, washed, and any wells with labeled PKIN complex are assayed. Data obtained using different concentrations of PKIN are used to calculate values for the number, affinity, and association of PKIN with the candidate molecules.

Alternatively, molecules interacting with PKIN are analyzed using the yeast two-hybrid system as described in Fields, S. and O. Song (1989) Nature 340:245-246, or using commercially available kits based on the two-hybrid system, such as the MATCHMAKER system (Clontech).

PKIN may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVII. Demonstration of PKIN Activity

Generally, protein kinase activity is measured by quantiying the phosphorylation of a protein substrate by PKIN in the presence of gamma-labeled ³²P-ATP. PKIN is incubated with the protein substrate, ³²P-ATP, and an appropriate kinase buffer. The ³²P incorporated into the substrate is separated from free ³²P-ATP by electrophoresis and the incorporated ³²P is counted using a radioisotope counter. The amount of incorporated ³²P is proportional to the activity of PKIN. A determination of the specific amino acid residue phosphorylated is made by phosphoamino acid analysis of the hydrolyzed protein.

In one alternative, protein kinase activity is measured by quantifying the transfer of gamma phosphate from adenosine triphosphate (ATP) to a serine, threonine or tyrosine residue in a protein substrate. The reaction occurs between a protein kinase sample with a biotinylated peptide substrate and gamma ³²P-ATP. Following the reaction, free avidin in solution is added for binding to the biotinylated ³²P-peptide product. The binding sample then undergoes a centrifugal ultrafiltration process with a membrane which will retain the product-avidin complex and allow passage of free gamma ³²P-ATP. The reservoir of the centrifuged unit containing the ³²P-peptide product as retentate is then counted in a scintillation counter. This procedure allows assay of any type of protein kinase sample, depending on the peptide substrate and kinase reaction buffer selected. This assay is provided in kit form (ASUA, Affinity Ultrafiltration Separation Assay, Transbio Corporation, Baltimore Md., U.S. Pat. No. 5,869,275). Suggested substrates and their respective enzymes are as follows: Histone H1 (Sigma) and p34^{cdc2} kinase, Annexin I, Angiotensin (Sigma) and EGF receptor kinase, Annexin II and src kinase, ERK1 & ERK2 substrates and MEK, and myelin basic protein and ERK (Pearson, J. D. et al. (1991) Methods Enzymol. 200:62-81).

In another alternative, protein kinase activity of PKIN is demonstrated in vitro in an assay containing PKIN, 50 μl of kinase buffer, 1 μg substrate, such as myelin basic protein (MBP) or synthetic peptide substrates, 1 mM DTT, 10 μg ATP, and 0.5 μCi [γ-³³P]ATP. The reaction is incubated at 30° C. for 30 minutes and stopped by pipetting onto P81 paper. The unincorporated [γ-³³P]ATP is removed by washing and the incorporated radioactivity is measured using a radioactivity scintillation counter. Alternatively, the reaction is stopped by heating to 100° C. in the presence of SDS loading buffer and visualized on a 12% SDS polyacrylamide gel by autoradiography. Incorporated radioactivity is corrected for reactions carried out in the absence of PKIN or in the presence of the inactive kinase, K38A.

In yet another alternative, adenylate kinase or guanylate kinase activity may be measured by the incorporation of ³²P from gamma-labeled ³²P -ATP into ADP or GDP using a gamma radioisotope counter. The enzyme, in a kinase buffer, is incubated together with the appropriate nucleotide mono-phosphate substrate (AMP or GMP) and ³²P-labeled ATP as the phosphate donor. The reaction is incubated at 37° C. and terminated by addition of trichloroacetic acid. The acid extract is neutralized and subjected to gel electrophoresis to separate the mono-, di-, and triphosphonucleotide fractions. The diphosphonucleotide fraction is cut out and counted. The radioactivity recovered is proportional to the enzyme activity.

In yet another alternative, other assays for PKIN include scintillation proximity assays (SPA), scintillation plate technology and filter binding assays. Useful substrates include recombinant proteins tagged with glutathione transferase, or synthetic peptide substrates tagged with biotin. Inhibitors of PKIN activity, such as small organic molecules, proteins or peptides, may be identified by such assays.

XVIII. Enhancement/Inhibition of Protein Kinase Activity

Agonists or antagonists of PKIN activation or inhibition may be tested using assays described in section XVII. Agonists cause an increase in PKIN activity and antagonists cause a decrease in PKIN activity.

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| | | Incyte | | Incyte |
|---|---|---|---|---|
| Incyte | Polypeptide | Polypeptide | Polynucleotide | Polynucleotide |
| Project ID | SEQ ID NO: | ID | SEQ ID NO: | ID |
| 2011384 | 1 | 2011384CD1 | 27 | 2011384CB1 |
| 2004888 | 2 | 2004888CD1 | 28 | 2004888CB1 |
| 2258952 | 3 | 2258952CD1 | 29 | 2258952CB1 |
| 7473244 | 4 | 7473244CD1 | 30 | 7473244CB1 |
| 1242491 | 5 | 1242491CD1 | 31 | 1242491CB1 |
| 2634875 | 6 | 2634875CD1 | 32 | 2634875CB1 |
| 3951059 | 7 | 3951059CD1 | 33 | 3951059CB1 |
| 7395890 | 8 | 7395890CD1 | 34 | 7395890CB1 |
| 7475546 | 9 | 7475546CD1 | 35 | 7475546CB1 |
| 7477076 | 10 | 7477076CD1 | 36 | 7477076CB1 |
| 1874092 | 11 | 1874092CD1 | 37 | 1874092CB1 |
| 4841542 | 12 | 4841542CD1 | 38 | 4841542CB1 |
| 7472695 | 13 | 7472695CD1 | 39 | 7472695CB1 |
| 7477966 | 14 | 7477966CD1 | 40 | 7477966CB1 |
| 7163416 | 15 | 7163416CD1 | 41 | 7163416CB1 |
| 7472822 | 16 | 7472822CD1 | 42 | 7472822CB1 |
| 7477486 | 17 | 7477486CD1 | 43 | 7477486CB1 |
| 3773709 | 18 | 3773709CD1 | 44 | 3773709CB1 |
| 7477204 | 19 | 7477204CD1 | 45 | 7477204CB1 |
| 3016969 | 20 | 3016969CD1 | 46 | 3016969CB1 |
| 063497 | 21 | 063497CD1 | 47 | 063497CB1 |
| 1625436 | 22 | 1625436CD1 | 48 | 1625436CB1 |
| 3330646 | 23 | 3330646CD1 | 49 | 3330646CB1 |
| 3562763 | 24 | 3562763CD1 | 50 | 3562763CB1 |
| 621293 | 25 | 621293CD1 | 51 | 621293CB1 |
| 7480774 | 26 | 7480774CD1 | 52 | 7480774CB1 |

TABLE 2

| | Incyte | | | |
|---|---|---|---|---|
| Polypeptide | Polypeptide | GenBank ID | Probability | |
| SEQ ID NO: | ID | NO: | Score | GenBank Homolog |
| 1 | 2011384CD1 | g404634 | 4.50E−60 | [Mus musculus] serine/threonine kinase |
| | | | | (Bielke, W. et al (1994) Gene 139 (2), 235-239) |
| | | g13540326 | 1.00E−159 | [fl] [Homo sapiens] serine/threonine kinase FKSG82 |
| 2 | 2004888CD1 | g2983205 | 2.70E−08 | [Aquifex aeolicus] ser/thr protein kinase |
| | | | | [Deckert, G. et al (1998) Nature 392 (6674), 353-358) |
| | | g13603881 | 0 | [fl] [Homo sapiens] serine/threonine kinase 31 |
| | | | | (Wang, P. J. et al, (2001) Nat. Genet. 27 (4), 422-426) |
| 3 | 2258952CD1 | g3766209 | 0 | [Mus musculus] IRE1 |
| | | | | (Wang, X. Z. et al (1998) EMBO J. 17 (19), 5708-5717) |
| 4 | 7473244CD1 | g2052189 | 0 | [Rattus norvegicus] serine/threonine kinase |
| 5 | 1242491CD1 | g2253010 | 8.40E−25 | [Arabidopsis thaliana] MAP3K delta-1 protein kinase |
| | | | | (Jouannic, S. et al (1999) Gene 229 (1-2), 171-181) |
| 6 | 2634875CD1 | g13194657 | 0 | [fl] [Homo sapiens] skeletal myosin light chain kinase |
| | | g165506 | 1.50E−272 | [Oryctolagus cuniculus] myosin light chain kinase (EC |
| | | | | 2.7.1.-) |
| | | | | (Herring, B. P. et al (1990) J. Biol. Chem. 265, 1724-1730) |
| 7 | 3951059CD1 | g3599507 | 5.00E−235 | [Mus musculus] rho/rac-interacting citron kinase short |
| | | | | isoform |
| | | | | (Di Cunto, F. et al (1998) J. Biol. Chem. 273 (45), |
| | | | | 29706-29711) |
| 8 | 7395890CD1 | g5815139 | 0 | [Mus musculus] nuclear body associated kinase 1a |
| 9 | 7475546CD1 | g3435114 | 1.80E−50 | [Homo sapiens] serine/threonine kinase ULK1 |
| | | | | (Kuroyanagi, H. et al (1998) Genomics 51 (1), 76-85) |
| 10 | 7477076CD1 | g854733 | 6.20E−198 | [Rattus norvegicus] casein kinase 1 gamma 1 isoform |
| 11 | 1874092CD1 | g2511715 | 4.00E−25 | [Arabidopsis thaliana] putative phosphatidylinositol-4- |
| | | | | phosphate |
| 12 | 4841542CD1 | g927732 | 3.30E−67 | [Saccharomyces cerevisiae] Snf1p: serine/threonine |
| | | | | protein kinase; |
| 13 | 7472695CD1 | g1498250 | 1.10E−53 | [Dictyostelium discoideum] myosin light chain kinase |
| | | | | (Tan, J. L. et al (1991) J. Biol. Chem. 266, 16044-16049) |
| | | g12830367 | 0 | [fl] [Homo sapiens] serine/threonine kinase 33 |
| 14 | 7477966CD1 | g3766209 | 0 | [Mus musculus] IRE1 |
| | | | | (Wang, X. Z. et al (1998) EMBO J. 17 (19), 5708-5717) |
| 15 | 7163416CD1 | g7649810 | 2.10E−135 | [Homo sapiens] protein kinase PAK5 |
| | | g11691855 | 0 | [fl] [Homo sapiens] pak5 protein |
| 16 | 7472822CD1 | g5081459 | 3.70E−241 | [Danio rerio] p55-related MAGUK protein DLG3 |
| 17 | 7477486CD1 | g3217028 | 0 | [5′ incom] [Homo sapiens] putative serine/threonine |
| | | | | protein kinase |
| | | | | (Stanchi, F. et al (2001) Yeast 18 (1), 69-80) |
| 18 | 3773709CD1 | g3986088 | 6.70E−78 | [Pyrococcus kodakaraensis] Glycerol Kinase |
| 19 | 7477204CD1 | g992672 | 7.30E−129 | [Homo sapiens] G protein-coupled receptor kinase GRK4- |
| | | | | beta |
| | | | | (Premont, R. T. et al (1996) J. Biol. Chem. 271 (11), |
| | | | | 6403-6410) |
| | | g4001826 | 0 | [fl] [Spermophilus tridecemlineatus] G protein-coupled |
| | | | | receptor kinase GRK7 |
| | | | | (Weiss, E. R. et al (1998) Mol. Vis. 4, 27) |
| 20 | 3016969CD1 | g4521278 | 4.70E−45 | [Homo sapiens] Trad |
| | | | | (Kawai, T. et al (1999) Gene 227 (2), 249-255) |
| 21 | 63497CD1 | g1213224 | 0 | [Rattus norvegicus] SNF1-related kinase |
| | | | | (Becker, W. et al (1996) Eur. J. Biochem. 235 (3), 736-743) |
| 22 | 1625436CD1 | g4096108 | 1.10E−252 | [Homo sapiens] proline rich calmodulin-dependent |
| | | | | protein kinase |
| | | g206152 | 0 | [fl] [Rattus norvegicus] calmodulin-dependent protein |
| | | | | kinase II gamma subunit (EC 2.7.1.37) |
| | | | | (Tobimatsu, T. et al (1988) J. Biol. Chem. 263, 16082-16086) |
| 23 | 3330646CD1 | g406058 | 0 | [Mus musculus] protein kinase |
| | | | | (Walden, P. D. et al (1993) Mol. Cell. Biol. 13, 7625-7635) |
| 24 | 3562763CD1 | g12830335 | 0 | [5′ incom] [Homo sapiens] bA550O8.2 (novel protein |
| | | | | kinase) |
| | | g1510182 | 9.80E−18 | [Mus musculus] cyclin-dependent kinase 5 |
| | | | | (Ishizuka, T. et al (1995) Gene 166 (2), 267-271) |
| 25 | 621293CD1 | g2649941 | 4.50E−23 | [Archaeoglobus fulgidus] adenylate kinase (adk) |
| | | | | (Klenk, H. P. et al (1997) Nature 390 (6658), 364-370) |
| 26 | 7480774CD1 | g2463542 | 0 | “[Homo sapiens] inositol 1,4,5-trisphosphate 3-kinase” |

TABLE 3

| SEQ | Incyte | Amino | Potential | Potential | | Analytical |
|---|---|---|---|---|---|---|
| ID | Polypeptide | Acid | Phosphorylation | Glycosylation | Signature Sequences, | Methods and |
| NO: | ID | Residues | Sites | Sites | Domains and Motifs | Databases |
| 1 | 2011384CD1 | 273 | Y12 Y23 T17 | | PROTEIN KINASE DOMAIN | BLAST_DOMO |
| | | | S144 T30 S31 | | DM00004\|P27448\|58-297: | |
| | | | S237 S253 | | R16-R255 | |
| | | | | | Eukaryotic protein kinase domain | HMMER_PFAM |
| | | | | | pkinase: | |
| | | | | | Y12-L267 | |
| | | | | | Protein kinases signatures and profile, | PROFILESCAN |
| | | | | | protein_kinase_tyr.prf: | |
| | | | | | Q111-G163 | |
| | | | | | Protein Kinase ATP binding site: | MOTIFS |
| | | | | | I18-K41 | |
| | | | | | Protein Kinase (serine/threonine): | MOTIFS |
| | | | | | L131-L143 | |
| | | | | | Tyrosine kinase catalytic domain | BLIMPS_PRINTS |
| | | | | | Signature: | |
| | | | | | PR00109: Y125-L143 Y193-S215 | |
| 2 | 2004888CD1 | 329 | S190 S50 S51 | | Eukaryotic protein kinase domain | HMMER_PFAM |
| | | | T141 Y302 | | pkinase: | |
| | | | | | P135-L228 | |
| | | | | | DM00004\|P54744\|13-263 PROTEIN KINASE | BLAST_DOMO |
| | | | | | DOMAIN: | |
| | | | | | P113-L228 (P = 1.1e−06) | |
| 3 | 2258952CD1 | 938 | S207 S299 S500 | N200 | PROTEIN KINASE DOMAIN | BLAST_DOMO |
| | | | S503 S580 S609 | | DM00004\|Q09499\|536-784: P534-A784 | |
| | | | S65 S714 S814 | | KINASE; THREONINE; ATP; SERINE; | BLAST_DOMO |
| | | | S852 S857 T116 | | DM06305\|Q09499\|786-924: V787-Y922 | |
| | | | T128 T147 T175 | | PROTEIN KINASE/ENDORIBONULCEASE PUTATIVE | BLAST_PRODOM |
| | | | T188 T202 T345 | | SERINE/THREONINE PROTEIN KINASE C41C4.4 | |
| | | | T55 T592 T658 | | CHROMOSOME II PRECURSOR TRANSFERASE | |
| | | | T84 T895 T905 | | PD152704: T170-L395, L61-E163 | |
| | | | T936 Y146 | | SERINE/THREONINE PROTEIN KINASE | BLAST_PRODOM |
| | | | | | PRECURSOR TRANSMEMBRANE SIGNAL | |
| | | | | | TRANSFERASE ATP-BINDING PROTEIN IRE1 | |
| | | | | | GLYCOPROTEIN | |
| | | | | | PD032590: W794-Y922 | |
| | | | | | Tyrosine kinase catalytic domain | BLIMPS_PRINTS |
| | | | | | PR00109: H639-I657, G694-L704, V716- | |
| | | | | | D738 | |
| | | | | | Protein kinases signatures and profile | PROFILESCAN |
| | | | | | protein_kinase_tyr.prf: | |
| | | | | | E625-G682 | |
| | | | | | Eukaryotic protein kinase domain | HMMER_PFAM |
| | | | | | pkinase: | |
| | | | | | F532-F793 | |
| | | | | | Protein_Kinase serine/theronine: | MOTIFS |
| | | | | | I645-I657 | |
| 4 | 7473244CD1 | 795 | S140 S2 S301 | N17 N331 | Protein kinases signatures and profile | PROFILESCAN |
| | | | S35 S423 S468 | N397 N398 | protein_kinase_tyr.prf: | |
| | | | S485 S486 S49 | | Y133-G210 | |
| | | | S524 S546 S609 | | Eukaryotic protein kinase domain | HMMER_PFAM |
| | | | S666 S671 S699 | | pkinase: | |
| | | | S705 S710 S776 | | Y60-M311 | |
| | | | T128 T19 T282 | | PROTEIN KINASE DOMAIN | BLAST_DOMO |
| | | | T324 T333 T437 | | DM00004\|P27448\|58-297: L62-L302 | |
| | | | T504 T511 T568 | | KINASE SERINE/THREONINEP ROTEIN PROTEIN | BLAST_PRODOM |
| | | | T581 T648 T657 | | TRANSFERASE ATP-BINDING SERINE/THREONINE | |
| | | | T676 T680 T82 | | PUTATIVE KIN1 EMK PAR1 | |
| | | | T9 | | PD004300: G682-L795 | |
| | | | | | SERINE/THREONINE KINASE | BLAST_PRODOM |
| | | | | | PD119193: I594-P665 | |
| | | | | | KINASE SERINE/THREONINE PROTEIN | BLAST_PRODOM |
| | | | | | SERINE/THREONINE PUTATIVE TRANSFERASE | |
| | | | | | ATP-BINDING PROTEIN EMK P78 CDC25C | |
| | | | | | PD008571: S412-S595 | |
| | | | | | KINASE SERINE/THREONINE PROTEIN PUTATIVE | BLAST_PRODOM |
| | | | | | SERINE/THREONINE TRANSFERASE ATP-BINDING | |
| | | | | | PROTEIN PAR1 KP78 EMK | |
| | | | | | PD005838: M311-R411 | |
| | | | | | Tyrosine kinase catalytic domain | BLIMPS_PRINTS |
| | | | | | PR00109: M136-V149, Y172-L190, V238- | |
| | | | | | Q260 | |
| | | | | | Protein_Kinase_ATP binding site: | MOTIFS |
| | | | | | I66-K89 | |
| | | | | | Protein_Kinase serine/theronine: | MOTIFS |
| | | | | | I178-L190 | |
| 5 | 1242491CD1 | 656 | S309 S42 S540 | N293 N424 | Eukaryotic protein kinase domain: | HMMER_PFAM |
| | | | S569 S583 S594 | N437 | L14-V257 | |
| | | | S654 T270 T303 | | Protein kinases signatures and profile: | PROFILESCAN |
| | | | T319 T366 T408 | | L99-Q151 | |
| | | | T439 T509 T526 | | Protein kinases ATP-binding region | MOTIFS |
| | | | T570 T609 T612 | | signature: | |
| | | | T623 T653 | | L14-K35 | |
| | | | | | Serine/Threonine protein kinases active- | MOTIFS |
| | | | | | site signature: | |
| | | | | | I119-L131 | |
| | | | | | Tyrosine kinase catalytic domain | BLIMPS_PRINTS |
| | | | | | signature | |
| | | | | | PR00109: M76-Q89, Y113-L131, A183-G205, | |
| | | | | | P232-S254 | |
| | | | | | PROTEIN KINASE DOMAIN | BLAST_DOMO |
| | | | | | DM00004\|P42679\|236-470: L14-P252 | |
| | | | | | DM00004\|I49621\|195-428: L14-P252 | |
| | | | | | DM00004\|I38044\|100-349: L13-P252 | |
| | | | | | DM00004\|Q05609\|553-797: L14-T197, L14- | |
| | | | | | T253 | |
| 6 | 2634875CD1 | 596 | S107 S143 S157 | N278 N416 | Eukaryotic protein kinase domain: | HMMER_PFAM |
| | | | S159 S184 S203 | | M285-L540 | |
| | | | S235 S397 S460 | | Tyrosine kinase catalytic domain | BLIMPS_PRINTS |
| | | | S586 S59 T17 | | signature | |
| | | | T224 T247 T301 | | PR00109: M359-V372, F396-C414, T463- | |
| | | | T320 T351 T379 | | D485 | |
| | | | T49 Y376 | | Protein kinases ATP-binding region | MOTIFS |
| | | | | | signature: | |
| | | | | | L291-K314 | |
| | | | | | Serine/Threonine protein kinases active- | MOTIFS |
| | | | | | site signature: | |
| | | | | | V402-C414 | |
| | | | | | KINASE MYOSIN LIGHT CHAIN SKELETAL | BLAST_PRODOM |
| | | | | | MUSCLE MLCK TRANSFERASE SERINE/THREONINE | |
| | | | | | CALMODULIN BINDING | |
| | | | | | PD036174: A95-M285 | |
| | | | | | PD027051: L540-V596 | |
| | | | | | PD029157: A2-R82, A2-S90 | |
| | | | | | PROTEIN KINASE DOMAIN | BLAST_DOMO |
| | | | | | DM00004\|P07313\|298-541: S287-A531 | |
| | | | | | DM00004\|JN0583\|727-969: K288-N530 | |
| | | | | | DM00004\|S07571\|5152-5396: E289-M529 | |
| | | | | | DM00004\|P53355\|15-257: E289-M529 | |
| 7 | 3951059CD1 | 497 | S140 S248 S308 | | Eukaryotic protein kinase domain: | HMMER_PFAM |
| | | | S361 S381 S386 | | F97-F360 | |
| | | | S410 S436 S445 | | Protein kinase C terminal domain: | HMMER_PFAM |
| | | | S490 S81 S93 | | S361-E390 | |
| | | | T279 T378 T83 | | Tyrosine kinase catalytic domain | BLIMPS_PRINTS |
| | | | | | signature | |
| | | | | | PR00109: M174-N187, S211-V229 | |
| | | | | | Protein kinases ATP-binding region | MOTIFS |
| | | | | | signature: | |
| | | | | | V103-K126 | |
| | | | | | Serine/Threonine protein kinases active- | MOTIFS |
| | | | | | site signature: | |
| | | | | | Y217-V229 | |
| | | | | | RHO/RACINTERACTING CITRON KINASE SHORT | BLAST_PRODOM |
| | | | | | ISOFORM | |
| | | | | | PD154232: S422-V468 | |
| | | | | | PD154360: M1-M43 | |
| | | | | | KINASE RHO ASSOCIATED COILED COIL | BLAST_PRODOM |
| | | | | | PROTEIN FORMING RHO/RAC INTERACTING | |
| | | | | | CITRON ALPHA | |
| | | | | | PD007970: Q32-D96 | |
| | | | | | PROTEIN KINASE DOMAIN | BLAST_DOMO |
| | | | | | DM00004\|Q09013\|83-336: V99-L349 | |
| | | | | | DM00004\|S42867\|75-498: S101-G241, | |
| | | | | | I258-S445 | |
| | | | | | DM00004\|S42864\|41-325: E98-G241, N249- | |
| | | | | | L349, D96-T153 | |
| | | | | | DM00004\|P38679\|238-527: L102-G241, | |
| | | | | | I258-L349, E86-A124 | |
| 8 | 7395890CD1 | 1171 | S121 S135 S178 | N140 N157 | Eukaryotic protein kinase domain: | HMMER_PFAM |
| | | | S180 S254 S27 | N271 N480 | Y199-P420, R498-V527 | |
| | | | S37 S405 S649 | N562 N579 | Tyrosine kinase catalytic domain | BLIMPS_PRINTS |
| | | | S773 S774 S783 | N786 N963 | signature | |
| | | | S788 S804 S865 | N978 N1012 | PR00109: K314-L332 | |
| | | | S970 T119 T172 | | Protein kinases ATP-binding region | MOTIFS |
| | | | T221 T431 T450 | | signature: | |
| | | | T483 T517 T839 | | L205-K228 | |
| | | | T867 T893 T995 | | Serine/Threonine protein kinases active- | MOTIFS |
| | | | T1022 S1027 | | site signature: | |
| | | | S1099 Y443 | | L320-L332 | |
| | | | Y468 | | PROTEIN KINASE NUCLEAR HOMEO DOMAIN | BLAST_PRODOM |
| | | | | | INTERACTING DNA-BINDING SERINE/THREONINE | |
| | | | | | PD141983: A573-C933 | |
| | | | | | PD150874: A993-I1171 | |
| | | | | | PROTEIN KINASE NUCLEAR SERINE/THREONINE | BLAST_PRODOM |
| | | | | | HOMEO DOMAIN INTERACTING DNA-BINDING | |
| | | | | | SERINE/THREONINE F20B6.8 | |
| | | | | | PD042899: L425-P574 | |
| | | | | | HOMEO DOMAIN INTERACTING PROTEIN KINASE | BLAST_PRODOM |
| | | | | | 2 DNA-BINDING NUCLEAR PROTEIN | |
| | | | | | PD184491: E872-P961 | |
| | | | | | PROTEIN KINASE DOMAIN | BLAST_DOMO |
| | | | | | DM00004\|P14680\|371-694: V201-P518 | |
| | | | | | DM00004\|Q09815\|519-804: E200-L473, | |
| | | | | | F500-T517 | |
| | | | | | DM00004\|P49657\|101-409: L205-P518 | |
| | | | | | DM00004\|Q09690\|700-985: E200-P444, | |
| | | | | | F500-P518 | |
| 9 | 7475546CD1 | 470 | S134 S146 S165 | N132 | Eukaryotic protein kinase domain: | HMMER_PFAM |
| | | | S217 S219 S227 | | F14-V270 | |
| | | | S256 S260 S339 | | Tyrosine kinase catalytic domain | BLIMPS_PRINTS |
| | | | S361 S406 S447 | | signature | |
| | | | S462 T105 T17 | | PR00109: M91-H104, F127-L145, L239-F261 | |
| | | | T37 T61 | | Protein kinases signatures and profile: | PROFILESCAN |
| | | | | | V113-P166 | |
| | | | | | Protein kinases ATP-binding region | MOTIFS |
| | | | | | signature: | |
| | | | | | L20-K44 | |
| | | | | | Serine/Threonine protein kinases active- | MOTIFS |
| | | | | | site signature: | |
| | | | | | I133-L145 | |
| | | | | | KINASE PROTEIN TRANSFERASE ATP BINDING | BLAST_PRODOM |
| | | | | | SERINE/THREONINE RECEPTOR TYROSINE | |
| | | | | | PRECURSOR TRANSMEMBRANE | |
| | | | | | PD000001: S176-P255, I15-F93, P237- | |
| | | | | | W269, F117-M164, L20-K34 | |
| | | | | | PROTEIN KINASE DOMAIN | BLAST_DOMO |
| | | | | | DM00004\|P53104\|26-315: P151-F261, E18- | |
| | | | | | E111, F117-S147 | |
| | | | | | DM00004\|S54788\|154-400: L20-S260 | |
| | | | | | DM00004\|P27448\|58-297: L16-R258 | |
| | | | | | DM00004\|P49673\|31-267: L20-I259 | |
| 10 | 7477076CD1 | 422 | S124 S150 S229 | | Eukaryotic protein kinase domain | HMMER_PFAM |
| | | | S96 T137 T14 | | pkinase: | |
| | | | T199 T214 T258 | | F44-E276 | |
| | | | T269 T273 T355 | | Protein kinases signatures and profile: | PROFILESCAN |
| | | | T374 T417 | | T140-P197 | |
| | | | | | Protein kinases ATP-binding region | MOTIFS |
| | | | | | signature: | |
| | | | | | I50-K73 | |
| | | | | | Serine/Threonine protein kinases active- | MOTIFS |
| | | | | | site signature: | |
| | | | | | L160-I172 | |
| | | | | | CASEIN KINASE I GAMMA ISOFORM | BLAST_PRODOM |
| | | | | | TRANSFERASE SERINE/THREONINE ATP BINDING | |
| | | | | | MULTIGENE | |
| | | | | | PD015080: F315-T393 | |
| | | | | | CASEIN KINASE I, GAMMA 1 ISOFORM EC | BLAST_PRODOM |
| | | | | | 2.7.1. CKI GAMMA TRANSFERASE | |
| | | | | | SERINE/THREONINE PROTEIN ATP BINDING | |
| | | | | | MULTIGENE | |
| | | | | | PD049080: M1-N43 | |
| | | | | | PROTEIN KINASE DOMAIN | BLAST_DOMO |
| | | | | | DM00004\|A56711\|46-303: V46-Y304 | |
| | | | | | DM00004\|C56711\|45-301: V46-Y304 | |
| | | | | | DM00004\|B56711\|48-303: V46-Y304 | |
| | | | | | DM00004\|D56406\|31-276: V46-V293 | |
| 11 | 1874092CD1 | 240 | S121 S132 S78 | | PROTEIN PHOSPHATIDYL INOSITOL 4- | BLAST_PRODOM |
| | | | T197 T84 | | PHOSPHATE 5-KINASE PUTATIVE T22C1.7 | |
| | | | | | ISOLOG ATPIP5K1 T4C15.16 | |
| | | | | | PD149995: L13-D204 | |
| 12 | 4841542CD1 | 594 | S108 S114 S293 | N542 N87 | KINASE PROTEIN TRANSFERASE ATP-BINDING | BLAST_PRODOM |
| | | | S297 S305 S306 | | SERINE/THREONINE PROTEIN PHOSPHORYLATION | |
| | | | S339 S343 S382 | | RECEPTOR TYROSINE PROTEIN PRECURSOR | |
| | | | S40 S427 S48 | | TRANSMEMBRANE | |
| | | | S489 S572 S88 | | PD000001: K3-S163, S178-F216, P236- | |
| | | | S99 T193 T255 | | W268 (P = 1.2e−09) | |
| | | | T259 T357 T477 | | PROTEIN KINASE DOMAIN | BLAST_DOMO |
| | | | T544 T582 Y425 | | DM00004\|P27448\|58-297: L22-L260 | |
| | | | | | DM00004\|P06782\|57-296: L22-L260 | |
| | | | | | DM00004\|JC1446\|20-261: T24-L260 | |
| | | | | | DM00004\|P54645\|17-258: E23-L260 | |
| | | | | | Tyrosine kinase catalytic domain | BLIMPS_PRINTS |
| | | | | | signature | |
| | | | | | PR00109: M95-S108, Y131-L149, V197- | |
| | | | | | H219 | |
| | | | | | Eukaryotic protein kinase domain | HMMER_PFAM |
| | | | | | pkinase: | |
| | | | | | Y19-V269 | |
| | | | | | Protein_Kinase_ATP | MOTIFS |
| | | | | | L25-K47 | |
| | | | | | Protein_Kinase_Serine/Threonine | MOTIFS |
| | | | | | V137-L149 | |
| 13 | 7472695CD1 | 473 | S128 S170 S208 | N172 N370 | Eukaryotic protein kinase domain | HMMER_PFAM |
| | | | S233 S255 S285 | N397 N54 | pkinase: | |
| | | | S30 S308 S347 | | Y75-L340 | |
| | | | S366 S379 S39 | | | |
| | | | S400 S432 S46 | | | |
| | | | T143 T29 T330 | | | |
| | | | T371 T399 T409 | | | |
| | | | T418 T469 T93 | | | |
| | | | | | PROTEIN KINASE DOMAIN | BLAST_DOMO |
| | | | | | DM00004\|S57347\|21-266: F77-T330 | |
| | | | | | DM00004\|S46283\|13-259: G78-A331 | |
| | | | | | DM00004\|S54788\|154-400: G78-A331 | |
| | | | | | DM00004\|P28583\|35-282: G78-A331 | |
| | | | | | KINASE PROTEIN TRANSFERASE ATP-BINDING | BLAST_PRODOM |
| | | | | | SERINE/THREONINE PROTEIN PHOSPHORYLATION | |
| | | | | | RECEPTOR TYROSINE PROTEIN PRECURSOR | |
| | | | | | TRANSMEMBRANE | |
| | | | | | PD000001: D197-L299, R79-D156 | |
| | | | | | Tyrosine kinase catalytic domain | BLIMPS_PRINTS |
| | | | | | PR00109: M151-D164, Y187-V205, C263- | |
| | | | | | S285, T143-R165 | |
| | | | | | Phosphorylase kinase family | BLIMPS_PRINTS |
| | | | | | PR101049: D164-I184 | |
| | | | | | Protein_Kinase_ATP | MOTIFS |
| | | | | | L81-K104 | |
| | | | | | Protein_Kinase_Serine/Threonine | MOTIFS |
| | | | | | I193-V205 | |
| | | | | | Protein_kinase_tyrosine.profile: | PROFILESCAN |
| | | | | | E173-A228 | |
| 14 | 7477966CD1 | 947 | S207 S299 S508 | N200 | Eukaryotic protein kinase domain | HMMER_PFAM |
| | | | S511 S589 S618 | | pkinase: | |
| | | | S65 S723 S823 | | F541-F802 | |
| | | | S861 S866 T116 | | | |
| | | | T128 T147 T175 | | | |
| | | | T188 T202 T345 | | | |
| | | | T55 T601 T667 | | | |
| | | | T84 T904 T914 | | | |
| | | | T945 Y146 | | | |
| | | | | | PROTEIN KINASE DOMAIN | BLAST_DOMO |
| | | | | | DM00004\|Q09499\|536-784: P543-A793 | |
| | | | | | DM00004\|P32361\|676-970: V546-Q714, | |
| | | | | | T722-A793 | |
| | | | | | do KINASE; THREONINE; ATP; SERINE; | BLAST_DOMO |
| | | | | | DM06305\|Q09499\|786-924: V796-Y931 | |
| | | | | | DM06305\|P32361\|972-1114: Q795-L928 | |
| | | | | | PROTEIN KINASE/ENDORIBONULCEASE PUTATIVE | BLAST_PRODOM |
| | | | | | SERINE/THREONINE PROTEIN KINASE C41C4.4 | |
| | | | | | CHROMOSOME II PRECURSOR TRANSFERASE | |
| | | | | | PD152704: T170-L395, L61-E163 | |
| | | | | | SERINE/THREONINE PROTEIN KINASE | BLAST_PRODOM |
| | | | | | PRECURSOR TRANSMEMBRANE SIGNAL | |
| | | | | | TRANSFERASE ATP-BINDING PROTEIN IRE1 | |
| | | | | | GLYCOPROTEIN | |
| | | | | | PD032590: W803-Y931 | |
| | | | | | Tyrosine kinase catalytic domain | BLIMPS_PRINTS |
| | | | | | signature | |
| | | | | | PR00109: H648-I666, G703-L713, V725- | |
| | | | | | D747 | |
| | | | | | Phosphorylase kinase family signature | BLIMPS_PRINTS |
| | | | | | PR01049: P794-R805 | |
| | | | | | Protein_Kinase_Serine/Threonine: | MOTIFS |
| | | | | | I654-I666 | |
| | | | | | protein_kinase_tyrosine.profile: | PROFILESCAN |
| | | | | | E634-G691 | |
| 15 | 7163416CD1 | 641 | S107 S135 S165 | N288 | Eukaryotic protein kinase domain | HMMER_PFAM |
| | | | S189 S248 S255 | | pkinase: | |
| | | | S276 S290 S332 | | L407-Y601 | |
| | | | S351 S429 S560 | | | |
| | | | S624 T106 T107 | | | |
| | | | T124 T212 T238 | | | |
| | | | T24 T322 T46 | | | |
| | | | T505 T580 T99 | | | |
| | | | | | PROTEIN KINASE DOMAIN | BLAST_DOMO |
| | | | | | DM00004\|P35465\|271-510: Y410-S628 | |
| | | | | | DM00004\|I49376\|270-509: K412-S628 | |
| | | | | | DM00004\|Q03497\|622-861: V411-S628 | |
| | | | | | DM00004\|P50527\|388-627: S409-S628 | |
| | | | | | KINASE SERINE/THREONINE PROTEIN | BLAST_PRODOM |
| | | | | | TRANSFERASE ATP-BINDING PROTEIN | |
| | | | | | PHOSPHORYLATION P21 ACTIVATED ACTIVATED | |
| | | | | | HOMOLOG SYNDROME | |
| | | | | | PD002852: I12-L44 (P = 3.0e−06) | |
| | | | | | Tyrosine kinase catalytic domain | BLIMPS_PRINTS |
| | | | | | signature | |
| | | | | | PR00109: M481-S494, Y516-L534, G563- | |
| | | | | | I573, V582-D604 | |
| | | | | | Protein_Kinase_ATP | MOTIFS |
| | | | | | I413-K436 | |
| 16 | 7472822CD1 | 576 | S109 S136 S220 | N334 | Guanylate kinase: | HMMER_PFAM |
| | | | S255 S266 S31 | | T404-N500 | |
| | | | S313 S318 S323 | | GUANYLATE KINASE | BLAST_DOMO |
| | | | S327 S336 S451 | | DM00755\|A57653\|370-570: P359-P570 | |
| | | | S505 S506 S8 | | DM00755\|I38757\|709-898: R369-P570 | |
| | | | T152 T213 T333 | | DM00755\|S32545\|1-196: R369-K556 | |
| | | | T353 T364 T403 | | DM00755\|P31007\|765-954: R369-P570 | |
| | | | T447 T470 T497 | | | |
| | | | T517 T557 Y440 | | | |
| | | | Y482 Y59 | | | |
| | | | | | PROTEIN DOMAIN SH3 KINASE GUANYLATE | BLAST_PRODOM |
| | | | | | TRANSFERASE ATP-BINDING REPEAT GMP | |
| | | | | | MEMBRANE | |
| | | | | | PD001338: T403-E496 | |
| | | | | | PROTEIN SH3 DOMAIN PERIPHERAL PLASMA | BLAST_PRODOM |
| | | | | | MEMBRANE CALMODULIN BINDING CASK | |
| | | | | | CAMGUK CALCIUM | |
| | | | | | PD008238: M1-I139 | |
| | | | | | PROTEIN MAGUK P55 SUBFAMILY MEMBER DISCS | BLAST_PRODOM |
| | | | | | LARGE HOMOLOG SH3 DOMAIN | |
| | | | | | PD152180: K230-R297 | |
| | | | | | PROTEIN MAGUK P55 SUBFAMILY MEMBER MPP3 | BLAST_PRODOM |
| | | | | | DISCS LARGE HOMOLOG SH3 | |
| | | | | | PD090357: S318-T403 | |
| | | | | | Guanylate kinase protein | BLIMPS_BLOCKS |
| | | | | | BL00856: V400-I420, D428-R475 | |
| | | | | | SH3 domain signature | BLIMPS_PRINTS |
| | | | | | PR00452: R284-R296, M231-P241, A252- | |
| | | | | | Q267 | |
| | | | | | PDZ domain (Also known as DHR or GLGF). | HMMER_PFAM |
| | | | | | PDZ: | |
| | | | | | I139-G219 | |
| | | | | | SH3 domain SH3: | HMMER_PFAM |
| | | | | | M231-R296 | |
| | | | | | Guanylate_Kinase: | MOTIFS |
| | | | | | T403-I420 | |
| | | | | | signal_cleavage: | SPSCAN |
| | | | | | M1-S31 | |
| 17 | 7477486CD1 | 794 | S130 S158 S19 | | PROTEIN KINASE DOMAIN | BLAST_DOMO |
| | | | S201 S291 S327 | | DM00004\|P34244\|82-359: I71-S291 | |
| | | | S357 S379 S420 | | DM00004\|JC1446\|20-261: R51-L292 | |
| | | | S443 S463 S512 | | DM00004\|P54645\|17-258: L52-L292 | |
| | | | S524 S571 S579 | | DM00004\|A53621\|18-258: L52-L292 | |
| | | | S602 S635 S659 | | KINASE PROTEIN TRANSFERASE ATP-BINDING | BLAST_PRODOM |
| | | | S684 S692 S715 | | SERINE/THREONINE PROTEIN PHOSPHORYLATION | |
| | | | S731 S774 T145 | | RECEPTOR TYROSINE PROTEIN PRECURSOR | |
| | | | T433 T488 T539 | | TRANSMEMBRANE | |
| | | | T591 | | PD000001: R42-L138, L144-A194 S209- | |
| | | | | | F247, I270-L302 | |
| | | | | | Tyrosine kinase catalytic domain | BLIMPS_PRINTS |
| | | | | | signature | |
| | | | | | PR00109: L126-V139, F162-L180, A228- | |
| | | | | | D250, I270-L292 | |
| | | | | | Eukaryotic protein kinase domain | HMMER_PFAM |
| | | | | | pkinase: | |
| | | | | | Y50-Y301 | |
| | | | | | Protein_Kinase_ATP | MOTIFS |
| | | | | | L56-K79 | |
| | | | | | Protein_Kinase_Serine/Threonine: | MOTIFS |
| | | | | | I168-L180 | |
| | | | | | Protein_kinase_tyrosine.profile: | PROFILESCAN |
| | | | | | K120-S201 | |
| 18 | 3773709CD1 | 504 | S117 S142 S152 | N131 N132 | XYLULOKINASE | BLAST-DOMO |
| | | | S169 S232 S339 | N178 N216 | DM02388\|P18157\|1-492: F20-M498 | |
| | | | T274 T333 T375 | | GLYCEROL 3PHOSPHOTRANSFERASE | BLAST-PRODOM |
| | | | T459 T6 T96 Y17 | | GLYCEROKINASE GK | |
| | | | | | PD001007: G239-A448 | |
| | | | | | SIMILAR TO GLYCEROL KINASE | BLAST-PRODOM |
| | | | | | PD130307: F20-K137 | |
| | | | | | FGGY family of carbohydrate kinase | BLIMPS-BLOCKS |
| | | | | | proteins | |
| | | | | | BL00933: F20-C43, Y54-P64, S159-N178, | |
| | | | | | T212-V248, G414-L429 | |
| | | | | | FGGY family of carbohydrate kinases | PROFILESCAN |
| | | | | | signatures prok_carb_kinases.prf: | |
| | | | | | P350-K409 | |
| | | | | | FGGY family of carbohydrate kinases | HMMER-PFAM |
| | | | | | FGGY: | |
| | | | | | L92-R122, L172-D224, F238-A448 | |
| | | | | | Fggy_Kinases_2: | MOTIFS |
| | | | | | A366-E386 | |
| 19 | 7477204CD1 | 553 | S187 S23 S36 | N418 N543 | PROTEIN KINASE DOMAIN | BLAST-DOMO |
| | | | S380 S399 S544 | | DM00004\|P32298\|157-401: F194-G440 | |
| | | | S58 T138 T139 | | RECEPTOR KINASE | BLAST- PRODOM |
| | | | T213 T348 T407 | | PD001932: K455-N531 | |
| | | | T537 T79 T85 | | Regulator of G-protein | BLIMPS-PFAM |
| | | | | | PF00615: F163-K179 V267-I280 | |
| | | | | | Tyrosine kinase catalytic domain | BLIMPS-PRINTS |
| | | | | | signature | |
| | | | | | PR00109: F419-S441, M268-Y281, H306- | |
| | | | | | L324, G352-L362, V372-Y394 | |
| | | | | | GPCR kinase signature | BLIMPS-PRINTS |
| | | | | | PR00717: Y172-Q184, K230-S248, P469- | |
| | | | | | I486, V492-F505, N507-T524 | |
| | | | | | Protein kinases signatures and profile | PROFILESCAN |
| | | | | | protein_kinase_tyr.prf: | |
| | | | | | R292-K345 | |
| | | | | | Regulator of G protein signaling domain | HMMER-PFAM |
| | | | | | RGS: | |
| | | | | | N55-P78, P162-L176 | |
| | | | | | Eukaryotic protein kinase domain | HMMER-PFAM |
| | | | | | pkinase: | |
| | | | | | F191-F454 | |
| | | | | | Protein_Kinase_Atp: | MOTIFS |
| | | | | | L197-K220 | |
| | | | | | Protein_Kinase_St: | MOTIFS |
| | | | | | I312-L324 | |
| 20 | 3016969CD1 | 871 | S121 S123 S135 | N211 | PROTEIN KINASE DOMAIN | BLAST-DOMO |
| | | | S153 S167 S203 | | DM00004\|S07571\|5152-5396: Q580-P812 | |
| | | | S293 S33 S353 | | Tyrosine | BLIMPS-PRINTS |
| | | | S409 S542 S557 | | PR00109: Y684-I702, T751-E773, I581- | |
| | | | S571 S597 S640 | | A603 | |
| | | | S652 S665 S667 | | Eukaryotic protein kinase domain | HMMER-PFAM |
| | | | S727 S81 T172 | | pkinase: | |
| | | | T417 T516 T526 | | F575-L827 | |
| | | | T76 T844 | | Protein_Kinase_Tyr: | MOTIFS |
| | | | | | I690-I702 | |
| 21 | 063497CD1 | 765 | S162 S181 S259 | N219 N289 | Eukaryotic protein kinase domain: | HMMER_PFAM |
| | | | S286 S291 S410 | N588 N618 | Y16-L269 | |
| | | | S431 S437 S472 | | Tyrosine kinase catalytic domain | BLIMPS_PRINTS |
| | | | S479 S495 S531 | | signature | |
| | | | S539 S544 S550 | | PR00109: L92-M105, Y129-F147, V238-L260 | |
| | | | S569 S576 S597 | | SNF1RELATED KINASE | BLAST_PRODOM |
| | | | S639 S646 S661 | | PD127501: Q346-D579 | |
| | | | S676 T172 T319 | | PD070820: T715-I765, E642-G693, I345- | |
| | | | T365 T474 T478 | | P370 | |
| | | | T50 T543 T622 | | ZK524.4 PROTEIN SNF1RELATED KINASE | BLAST_PRODOM |
| | | | T623 T684 T714 | | PD156028: I282-I345 | |
| | | | T716 | | KINASE TRANSFERASE ATP BINDING SERINE/ | BLAST_PRODOM |
| | | | | | THREONINE PHOSPHORYLATION RECEPTOR | |
| | | | | | TYROSINE TRANSMEMBRANE | |
| | | | | | PD000001: L18-V145, V238-W268, G168- | |
| | | | | | F215 | |
| | | | | | PROTEIN KINASE DOMAIN | BLAST_DOMO |
| | | | | | DM00004\|P27448\|58-297: K20-L260 | |
| | | | | | DM00004\|I48609\|55-294: K20-L260 | |
| | | | | | DM00004\|Q05512\|55-294: K20-L260 | |
| | | | | | DM00004\|JC1446\|20-261: L18-L260 | |
| | | | | | Protein kinases ATP-binding region | MOTIFS |
| | | | | | signature: | |
| | | | | | L22-K45 | |
| | | | | | Serine/Threonine protein kinases active- | MOTIFS |
| | | | | | site signature: | |
| | | | | | V135-F147 | |
| 22 | 1625436CD1 | 588 | S109 S355 S356 | N313 N394 | Eukaryotic protein kinase domain: | HMMER_PFAM |
| | | | S36 S427 S433 | N407 N424 | Y14-V272 | |
| | | | S51 S557 S79 | | Protein kinases signatures and profile: | PROFILESCAN |
| | | | T262 T383 T408 | | F85-E167 | |
| | | | T409 T410 T47 | | Tyrosine kinase catalytic domain | BLIMPS_PRINTS |
| | | | T488 T94 | | signature | |
| | | | | | PR00109: H126-L144 | |
| | | | | | KINASE II CALCIUM/CALMODULIN DEPENDENT | BLAST_PRODOM |
| | | | | | SUBUNIT TRANSFERASE SERINE/THREONINE | |
| | | | | | PD004250: E500-Q588 | |
| | | | | | PD001779: R456-V499, V272-S329, T396- | |
| | | | | | A417 | |
| | | | | | PROTEIN KINASE DOMAIN | BLAST_DOMO |
| | | | | | DM00004\|P11798\|15-261: L16-A263 | |
| | | | | | DM00004\|JU0270\|16-262: E18-A263 | |
| | | | | | DM00004\|A44412\|16-262: E18-A263 | |
| | | | | | DM00004\|S57347\|21-266: L20-T262 | |
| | | | | | Protein kinases ATP-binding region | MOTIFS |
| | | | | | signature: | |
| | | | | | L20-K43 | |
| | | | | | Serine/Threonine protein kinases active- | MOTIFS |
| | | | | | site signature: | |
| | | | | | I132-L144 | |
| 23 | 3330646CD1 | 1798 | S74 S92 S1084 | N142 N1193 | Eukaryotic protein kinase domain: | HMMER_PFAM |
| | | | S108 S130 S1100 | N1252 | F512-F785 | |
| | | | S166 S171 S1205 | N1293 | | |
| | | | S200 S204 S1195 | | | |
| | | | S230 S253 S1214 | | | |
| | | | S281 S480 S1230 | | | |
| | | | S503 S508 S1225 | | | |
| | | | S533 S775 S1229 | | | |
| | | | S806 S811 S1272 | | | |
| | | | S817 S825 S1256 | | | |
| | | | S846 S854 S1332 | | | |
| | | | S860 S874 S1337 | | | |
| | | | S909 S914 S1418 | | | |
| | | | S931 S1425 | | | |
| | | | S1429 | | | |
| | | | S1447 S1459 | | | |
| | | | S1491 S1503 | | | |
| | | | S1504 S1541 | | | |
| | | | S1650 S1657 | | | |
| | | | S1660 S1671 | | | |
| | | | S1698 S1717 | | | |
| | | | S1771 T266 T506 | | | |
| | | | T1014 T514 T565 | | | |
| | | | T1036 T581 T729 | | | |
| | | | T1040 T759 T786 | | | |
| | | | T1117 T815 T82 | | | |
| | | | T1189 T871 T916 | | | |
| | | | T1236 T925 T949 | | | |
| | | | T1244 T1424 | | | |
| | | | T1480 T1675 | | | |
| | | | T1765 | | | |
| | | | | | PDZ domain: | HMMER_PFAM |
| | | | | | P1104-L1191 | |
| | | | | | Protein kinases signatures and profile: | PROFILESCAN |
| | | | | | F579-M659 | |
| | | | | | Tyrosine kinase catalytic domain | BLIMPS_PRINTS |
| | | | | | signature | |
| | | | | | PR00109: M589-K602, Y625-I643, V706- | |
| | | | | | D728 | |
| | | | | | MICROTUBULE ASSOCIATED TESTIS SPECIFIC | BLAST_PRODOM |
| | | | | | SERINE/THREONINE PROTEIN KINASE | |
| | | | | | PD142315: H1313-T1798 | |
| | | | | | PD135564: V61-Y320, L1151-P1363 | |
| | | | | | PD182663: E863-H1139 | |
| | | | | | PROTEIN KINASE SERINE/THREONINE KIN4 | BLAST_PRODOM |
| | | | | | MICROTUBULE ASSOCIATED TESTIS SPECIFIC | |
| | | | | | PD041650: K321-D511 | |
| | | | | | PROTEIN KINASE DOMAIN | BLAST_DOMO |
| | | | | | DM00004\|A54602\|455-712: T514-G772 | |
| | | | | | DM08046\|P05986\|1-397: S508-K658, | |
| | | | | | V685-E829, D268-P291 | |
| | | | | | DM00004\|S42867\|75-498: I515-T666, | |
| | | | | | H672-F813 | |
| | | | | | DM00004\|S42864\|41-325: E513-K658, | |
| | | | | | H672-T773 | |
| | | | | | Serine/Threonine protein kinases active- | MOTIFS |
| | | | | | site signature: | |
| | | | | | I631-I643 | |
| 24 | 3562763CD1 | 362 | S123 S157 S25 | N110 N165 | transmembrane domain: | HMMER |
| | | | S325 S81 T164 | | A263-D283 | |
| | | | T197 T260 T280 | | Eukaryotic protein kinase domain: | HMMER_PFAM |
| | | | T286 T324 T353 | | Y30-L351 | |
| | | | | | Protein kinases signatures and profile: | PROFILESCAN |
| | | | | | T164-G218 | |
| | | | | | Tyrosine kinase catalytic domain | BLIMPS_PRINTS |
| | | | | | signature | |
| | | | | | PR00109: M143-L156, F178-I196, M326- | |
| | | | | | A348 | |
| | | | | | PROTEIN KINASE DOMAIN | BLAST_DOMO |
| | | | | | DM00004\|Q02723\|16-259: K111-V215, | |
| | | | | | N232-V304 | |
| | | | | | DM00004\|A54602\|455-712: N110-L316, I36- | |
| | | | | | I61 | |
| | | | | | DM00004\|P23573\|10-277: L139-K214, | |
| | | | | | E35-L102, F248-A348 | |
| | | | | | DM00004\|A57459\|417-662: Y138-S325, E35- | |
| | | | | | L73 | |
| | | | | | Protein kinases ATP-binding region | MOTIFS |
| | | | | | signature: | |
| | | | | | I36-K59 | |
| | | | | | Serine/Threonine protein kinases active- | MOTIFS |
| | | | | | site signature: | |
| | | | | | I184-I196 | |
| 25 | 621293CD1 | 275 | | | Adenylate kinase: | HMMER_PFAM |
| | | | | | L69-P205 | |
| | | | | | Adenylate kinase proteins. | BLIMPS_BLOCKS |
| | | | | | BL00113: L68-L84, N92-R135, C141-L155 | |
| | | | | | Adenylate kinase signature | BLIMPS_PRINTS |
| | | | | | PR00094: L68-A81, G96-G110, W146-N162 | |
| 26 | 7480774CD1 | 660 | S104 S106 S167 | N177 | INOSITOL 3 KINASE 1D MYOINOSITOL | BLAST_PRODOM |
| | | | S199 S226 S325 | | TRISPHOSPHATE 5 TRISPHOSPHATE IP3K IP3 | |
| | | | S338 S339 S343 | | TRANSFERASE KINASE CALMODULIN BINDING | |
| | | | S355 S381 S458 | | PD010031: Q446-Q659, P377-Q442 | |
| | | | S46 S629 S96 | | CALMODULIN-BINDING DOMAIN | BLAST_DOMO |
| | | | T117 T151 T160 | | DM07435\|P42335\|210-672: E315-Q659 | |
| | | | T183 T210 T468 | | DM07435\|P23677\|1-461: G261-Q659 | |
| | | | T500 T83 T90 | | | |
| | | | T99 | | | |

TABLE 4

| Polynucleotide | Incyte | Sequence | Selected | | | |
|---|---|---|---|---|---|---|
| SEQ ID NO: | Polynucleotide ID | Length | Fragment(s) | Sequence Fragments | 5′ Position | 3′ Position |
| 27 | 2011384CB1 | 822 | 282-377 | 6829315H1 | 44 | 743 |
| | | | | (SINTNOR01) | | |
| | | | | g2954208 | 1 | 282 |
| 28 | 2004888CB1 | 1376 | 1349-1376, 499-635 | 5545302T6 | 713 | 1376 |
| | | | | (TESTNOC01) | | |
| | | | | 674588R6 | 517 | 1256 |
| | | | | (CRBLNOT01) | | |
| | | | | 5562195F8 | 1 | 644 |
| | | | | (BRSTDIT01) | | |
| 29 | 2258952CB1 | 3468 | 1-983, 1461-1908, | 3219989H1 | 3223 | 3468 |
| | | | 3369-3468 | (COLNNON03) | | |
| | | | | 2258952T6 | 2757 | 3353 |
| | | | | (OVARTUT01) | | |
| | | | | FL2258952_g7458755_{—} | 33 | 2849 |
| | | | | 000012_g3766209 | | |
| | | | | 7126256H1 | 2527 | 3076 |
| | | | | (COLNDIY01) | | |
| | | | | g1633937 | 2718 | 3385 |
| | | | | 7677920H1 | 1 | 601 |
| | | | | (NOSETUE01) | | |
| 30 | 7473244CB1 | 2831 | 1-243, 834-1782 | 2660853T6 | 2249 | 2831 |
| | | | | (LUNGTUT09) | | |
| | | | | 5216205F6 | 1789 | 2681 |
| | | | | (BRSTNOT35) | | |
| | | | | 6854507F8 | 763 | 1471 |
| | | | | (BRAIFEN08) | | |
| | | | | 55057226H1 | 354 | 1145 |
| | | | | 5911008F6 | 1299 | 1988 |
| | | | | (BRAIFEN05) | | |
| | | | | 2074751F6 | 1626 | 2118 |
| | | | | (ISLTNOT01) | | |
| | | | | 6881535J1 | 1 | 582 |
| | | | | (BRAHTDR03) | | |
| 31 | 1242491CB1 | 2693 | 1-317, 2569-2693 | 70006068D1 | 1296 | 1838 |
| | | | | 70006347D1 | 1162 | 1747 |
| | | | | 7934296H1 | 2109 | 2693 |
| | | | | (COLNDIS02) | | |
| | | | | 70003021D1 | 1740 | 2337 |
| | | | | 7226035H1 | 725 | 1187 |
| | | | | (LUNGTMC01) | | |
| | | | | 5755513H1 | 672 | 1102 |
| | | | | (LUNGNOT35) | | |
| | | | | 70004229D1 | 1874 | 2338 |
| | | | | 55052947H1 | 1 | 694 |
| 32 | 2634875CB1 | 2973 | 1-1353, 2203-2560 | 4009430F6 | 959 | 1432 |
| | | | | (MUSCNOT10) | | |
| | | | | 5168601H1 | 1691 | 1965 |
| | | | | (MUSCDMT01) | | |
| | | | | 5672440H1 | 2213 | 2414 |
| | | | | (MUSLTDT01) | | |
| | | | | 6903523H1 | 1833 | 2344 |
| | | | | (MUSLTDR02) | | |
| | | | | 55052146J1 | 1475 | 1654 |
| | | | | 6217472F6 | 2263 | 2973 |
| | | | | (MUSCDIT06) | | |
| | | | | 3585116F6 | 623 | 1126 |
| | | | | (293TF4T01) | | |
| | | | | GBI.g7242443_{—} | 1059 | 1585 |
| | | | | 000006.edit | | |
| | | | | 55052619J1 | 1 | 807 |
| | | | | 2634875H1 | 1521 | 1764 |
| | | | | (BONTNOT01) | | |
| 33 | 3951059CB1 | 2066 | 532-772, 1830-1886, | 6882814J1 | 1489 | 2066 |
| | | | 1966-2066 | (BRAHTDR03) | | |
| | | | | 55058330J1 | 396 | 1316 |
| | | | | FL452484_00001 | 1 | 970 |
| | | | | 71179403V1 | 1052 | 1745 |
| 34 | 7395890CB1 | 3975 | 1-326, 3951-3975, | 6771964H1 | 715 | 1432 |
| | | | 2980-3355, | (BRAUNOR01) | | |
| | | | 3666-3731, 1813-2074, | 6770122H1 | 1471 | 2040 |
| | | | 1066-1098 | (BRAUNOR01) | | |
| | | | | 6771964J1 | 2028 | 2713 |
| | | | | (BRAUNOR01) | | |
| | | | | 7393659H1 | 186 | 799 |
| | | | | (BRABDIE02) | | |
| | | | | 55052405H1 | 1 | 218 |
| | | | | 2570554R6 | 2495 | 3012 |
| | | | | (HIPOAZT01) | | |
| | | | | 7660364H1 | 1861 | 2459 |
| | | | | (OVARNOE02) | | |
| | | | | FL034583_00001 | 2778 | 3584 |
| | | | | 7395271H1 | 256 | 896 |
| | | | | (BRABDIE02) | | |
| | | | | 6200064H1 | 2715 | 3162 |
| | | | | (PITUNON01) | | |
| | | | | 7395911H1 | 896 | 1481 |
| | | | | (BRABDIE02) | | |
| | | | | GNN.g8439948_000007. | 3181 | 3975 |
| | | | | edit2.comp | | |
| | | | | 6873077H1 | 1327 | 1999 |
| | | | | (BRAGNON02) | | |
| 35 | 7475546CB1 | 1918 | 1-46, 658-1061 | 6623984J1 | 655 | 1287 |
| | | | | (UTRMTMR02) | | |
| | | | | 7192851H2 | 497 | 1107 |
| | | | | (BRATDIC01) | | |
| | | | | 6810083J1 | 1254 | 1918 |
| | | | | (SKIRNOR01) | | |
| | | | | 7013748H1 | 1 | 580 |
| | | | | (KIDNNOC01) | | |
| 36 | 7477076CB1 | 1689 | 1-66 | 7190770H1 | 216 | 771 |
| | | | | (BRATDIC01) | | |
| | | | | 55051332H1 | 1 | 282 |
| | | | | 6819441H1 | 1077 | 1689 |
| | | | | (OVARDIR01) | | |
| | | | | 7758313J1 | 558 | 922 |
| | | | | (SPLNTUE01) | | |
| | | | | GNN: g807680_edit | 820 | 1476 |
| 37 | 1874092CB1 | 1054 | 1-30 | 1874092F6 | 604 | 1054 |
| | | | | (LEUKNOT02) | | |
| | | | | 7315561H1 | 1 | 633 |
| | | | | (SYNODIN02) | | |
| 38 | 4841542CB1 | 3360 | 1-172, 2484-2523, | 71224917V1 | 2797 | 3360 |
| | | | 650-1457, | 70858292V1 | 2345 | 3032 |
| | | | 2247-2417 | 8045106H1 | 1719 | 2379 |
| | | | | (OVARTUE01) | | |
| | | | | 7617315J1 | 1036 | 1632 |
| | | | | (KIDNTUE01) | | |
| | | | | 7609838J1 | 783 | 1346 |
| | | | | (KIDCTME01) | | |
| | | | | 70856122V1 | 2494 | 3142 |
| | | | | 71225608V1 | 1597 | 2126 |
| | | | | 55053856H1 | 1 | 826 |
| 39 | 7472695CB1 | 2240 | 1-20, 101-131, | 7191541F6 | 1 | 906 |
| | | | 704-1001 | (BRATDIC01) | | |
| | | | | 71872279V1 | 911 | 1501 |
| | | | | 4211726T8 | 1466 | 2181 |
| | | | | (BRONDIT01) | | |
| | | | | 71870527V1 | 1717 | 2240 |
| | | | | 71870095V1 | 669 | 1374 |
| | | | | 2013786T6 | 1551 | 2217 |
| | | | | (TESTNOT03) | | |
| 40 | 7477966CB1 | 3340 | 1-980, 1504-1710, | 1513994T6 | 2768 | 3340 |
| | | | 3315-3340 | (PANCTUT01) | | |
| | | | | 6802962H1 | 2241 | 2824 |
| | | | | (COLENOR03) | | |
| | | | | 55052773H1 | 1376 | 2254 |
| | | | | 1513994F6 | 2155 | 2776 |
| | | | | (PANCTUT01) | | |
| | | | | 55052765H1 | 894 | 1745 |
| | | | | 7607337J1 | 594 | 1258 |
| | | | | (COLRTUE01) | | |
| | | | | 6802518H1 | 551 | 858 |
| | | | | (COLENOR03) | | |
| | | | | 7677920H1 | 1 | 598 |
| | | | | (NOSETUE01) | | |
| 41 | 7163416CB1 | 2539 | 1-228, 913-1225, | 7715351J1 | 1 | 649 |
| | | | 1994-2539 | (SINTFEE02) | | |
| | | | | 1625532H1 | 1779 | 1993 |
| | | | | (COLNPOT01) | | |
| | | | | 7163416F8 | 1888 | 2539 |
| | | | | (PLACNOR01) | | |
| | | | | 7701682J1 | 815 | 1434 |
| | | | | (PENHTUE02) | | |
| | | | | 7715351H1 | 399 | 1037 |
| | | | | (SINTFEE02) | | |
| | | | | 7077243H1 | 1306 | 1979 |
| | | | | (BRAUTDR04) | | |
| 42 | 7472822CB1 | 2377 | 2341-2377, 1093-1463, | 71982976V1 | 913 | 1546 |
| | | | 1625-2081 | 71983661V1 | 793 | 1520 |
| | | | | 71986606V1 | 1494 | 2168 |
| | | | | 55052941J1 | 1 | 886 |
| | | | | 71983943V1 | 1551 | 2193 |
| | | | | 71983660V1 | 1642 | 2377 |
| 43 | 7477486CB1 | 2897 | 2698-2763, 1-365, | 4029722F8 | 2042 | 2584 |
| | | | 2314-2623, | (BRAINOT23) | | |
| | | | 1516-1614, 2804-2897 | 6910737R6 | 462 | 1370 |
| | | | | (PITUDIR01) | | |
| | | | | 7237528H1 | 2348 | 2897 |
| | | | | (BRAINOY02) | | |
| | | | | 7674962H2 | 125 | 589 |
| | | | | (NOSETUE01) | | |
| | | | | 71982594V1 | 1386 | 1991 |
| | | | | 6629715R6 | 637 | 1476 |
| | | | | (HEALDIR01) | | |
| | | | | GNN.g6165121_004.edit | 1 | 506 |
| | | | | 6950253H1 | 1480 | 2176 |
| | | | | (BRAITDR02) | | |
| 44 | 3773709CB1 | 3361 | 1-168, 1479-1982, | 6938382F6 | 116 | 850 |
| | | | 3336-3361 | (FTUBTUR01) | | |
| | | | | 4383108H1 | 1 | 257 |
| | | | | (BRAVUTT02) | | |
| | | | | 7365206H1 | 2019 | 2580 |
| | | | | (OVARDIC01) | | |
| | | | | 55024481H1 | 791 | 1462 |
| | | | | (PKINDNV08) | | |
| | | | | 4119492H1 | 3104 | 3361 |
| | | | | (BRSTTUT25) | | |
| | | | | 70783206V1 | 1969 | 2579 |
| | | | | 3432983T6 | 2555 | 3217 |
| | | | | (SKINNOT04) | | |
| | | | | 70782455V1 | 1361 | 2005 |
| | | | | 70143324V1 | 2631 | 3219 |
| | | | | 70784860V1 | 1463 | 2006 |
| 45 | 7477204CB1 | 1662 | 854-1662, 1-807 | GNN.g8139716_edit | 1 | 1662 |
| 46 | 3016969CB1 | 3225 | 1-916, 1154-1362, | 71873834V1 | 1555 | 2122 |
| | | | 3144-3225 | 5751549F8 | 2153 | 2740 |
| | | | | (LUNGNOT35) | | |
| | | | | 7718401J1 | 1341 | 2100 |
| | | | | (SINTFEE02) | | |
| | | | | 7354408H1 | 2779 | 3225 |
| | | | | (HEARNON03) | | |
| | | | | 71872969V1 | 1969 | 2707 |
| | | | | 71875134V1 | 885 | 1440 |
| | | | | 3016969T6 | 2532 | 3211 |
| | | | | (MUSCNOT07) | | |
| | | | | 6200811F6 | 808 | 1403 |
| | | | | (PITUNON01) | | |
| | | | | 55052669H1 | 1 | 852 |
| 47 | 063497CB1 | 4772 | 1-431, 4420-4540, | 6581829H1 | 2823 | 3464 |
| | | | 2098-2130, | (HEACDIC01) | | |
| | | | 3522-3599, 2875-3036 | 7199634H1 | 602 | 1153 |
| | | | | (LUNGFER04) | | |
| | | | | 6936880H1 | 3000 | 3714 |
| | | | | (FTUBTUR01) | | |
| | | | | 1449223H1 | 4029 | 4248 |
| | | | | (PLACNOT02) | | |
| | | | | 4787168H1 | 3705 | 3964 |
| | | | | (BRATNOT03) | | |
| | | | | 7714789H1 | 1198 | 1849 |
| | | | | (SINTFEE02) | | |
| | | | | 7714789J1 | 4189 | 4772 |
| | | | | (SINTFEE02) | | |
| | | | | 063497H1 | 1661 | 1880 |
| | | | | (PLACNOB01) | | |
| | | | | 8025257J1 | 1 | 702 |
| | | | | (ENDMUNE01) | | |
| | | | | 7381417H1 | 1790 | 2359 |
| | | | | (ENDMUNE01) | | |
| | | | | 4351289H1 | 3884 | 4222 |
| | | | | (CONFTMT01) | | |
| | | | | 5068175H1 | 3675 | 3946 |
| | | | | (PANCNOT23) | | |
| | | | | 7380657H1 | 772 | 1305 |
| | | | | (ENDMUNE01) | | |
| | | | | 4051307H1 | 2689 | 2972 |
| | | | | (SINTNOT18) | | |
| | | | | 7627517J1 | 2393 | 2919 |
| | | | | (GBLADIE01) | | |
| | | | | 7629590H1 | 1953 | 2559 |
| | | | | (GBLADIE01) | | |
| 48 | 1625436CB1 | 1880 | 948-1167 | 5772228H1 | 844 | 1420 |
| | | | | (BRAINOT20) | | |
| | | | | 72285173V1 | 673 | 1148 |
| | | | | 7353062H1 | 1 | 610 |
| | | | | (HEARNON03) | | |
| | | | | 7154515H1 | 1164 | 1839 |
| | | | | (BRAMNOA01) | | |
| | | | | 6764194H1 | 1370 | 1880 |
| | | | | (BRAUNOR01) | | |
| | | | | 72284772V1 | 491 | 1135 |
| 49 | 3330646CB1 | 5747 | 1-1738, 2291-2733, | 8178538H2 | 5053 | 5722 |
| | | | 3677-4763 | (EYERNON01) | | |
| | | | | 7218734H1 | 4882 | 5570 |
| | | | | (COLNTMC01) | | |
| | | | | 8013776H1 | 4245 | 4904 |
| | | | | (HEARNOC04) | | |
| | | | | 8006864H1 | 442 | 1064 |
| | | | | (PENIFEC01) | | |
| | | | | 7711762H2 | 688 | 1292 |
| | | | | (TESTTUE02) | | |
| | | | | 55124907H1 | 1301 | 2151 |
| | | | | 8009629H1 | 3681 | 4314 |
| | | | | (NOSEDIC02) | | |
| | | | | 7054991H1 | 5099 | 5747 |
| | | | | (BRALNON02) | | |
| | | | | 55124907J1 | 1250 | 2101 |
| | | | | 8267426H1 | 2739 | 3511 |
| | | | | (MIXDUNF03) | | |
| | | | | 8054655J1 | 2905 | 3529 |
| | | | | (ESOGTUE01) | | |
| | | | | 7930953H1 | 4339 | 4966 |
| | | | | (COLNDIS02) | | |
| | | | | 7978939H1 | 1 | 504 |
| | | | | (LSUBDMC01) | | |
| | | | | 7719236J1 | 2085 | 2746 |
| | | | | (SINTFEE02) | | |
| | | | | 60215898V1 | 2234 | 2776 |
| | | | | 6779321J1 | 3439 | 4230 |
| | | | | (OVARDIR01) | | |
| 50 | 3562763CB1 | 3418 | 1564-1627, 1-376, | 55053205H1 | 523 | 1210 |
| | | | 975-1073, | 7321924H1 | 1843 | 2392 |
| | | | 3066-3418 | (NOSETUE01) | | |
| | | | | 7278180H1 | 2873 | 3418 |
| | | | | (BMARTXE01) | | |
| | | | | 400518R6 | 873 | 1430 |
| | | | | (PITUNOT02) | | |
| | | | | 6816641J1 | 1297 | 1981 |
| | | | | (ADRETUR01) | | |
| | | | | g2963935 | 1 | 383 |
| | | | | 55143790J1 | 2257 | 3143 |
| | | | | 55067380J2 | 314 | 579 |
| | | | | 55143774J1 | 2577 | 3148 |
| 51 | 621293CB1 | 995 | 1-372, 410-468 | 72335268V1 | 1 | 508 |
| | | | | 71870548V1 | 477 | 994 |
| 52 | 7480774CB1 | 2459 | 1664-2459, 1-110 | 71440281V1 | 685 | 1345 |
| | | | | 71438714V1 | 652 | 1226 |
| | | | | 7082565H1 | 1 | 688 |
| | | | | (STOMTMR02) | | |
| | | | | 71432228V1 | 1798 | 2459 |
| | | | | 71431941V1 | 1257 | 1972 |
| | | | | 6472388H1 | 1352 | 1985 |
| | | | | (PLACFEB01) | | |

TABLE 5

| Polynucleotide | Incyte | |
|---|---|---|
| SEQ ID NO: | Project ID | Representative Library |
| 27 | 2011384CB1 | SINTNOR01 |
| 28 | 2004888CB1 | TESTNOT03 |
| 29 | 2258952CB1 | COLENOR03 |
| 30 | 7473244CB1 | ISLTNOT01 |
| 31 | 1242491CB1 | LUNGNOT02 |
| 32 | 2634875CB1 | MUSCNOT07 |
| 33 | 3951059CB1 | DRGCNOT01 |
| 34 | 7395890CB1 | BRABDIE02 |
| 35 | 7475546CB1 | CORPNOT02 |
| 36 | 7477076CB1 | BRATDIC01 |
| 37 | 1874092CB1 | LEUKNOT02 |
| 38 | 4841542CB1 | KIDNNOT05 |
| 39 | 7472695CB1 | TESTNOT03 |
| 40 | 7477966CB1 | COLENOR03 |
| 41 | 7163416CB1 | ESOGTME01 |
| 42 | 7472822CB1 | BRABDIR03 |
| 43 | 7477486CB1 | BRAITDR03 |
| 44 | 3773709CB1 | SINTNOR01 |
| 46 | 3016969CB1 | COLNNOT41 |
| 47 | 063497CB1 | ENDMUNE01 |
| 48 | 1625436CB1 | BRACNOK02 |
| 49 | 3330646CB1 | HNT2AGT01 |
| 50 | 3562763CB1 | BRAHNOE01 |
| 51 | 621293CB1 | KIDNNOT09 |
| 52 | 7480774CB1 | BLADTUT02 |

TABLE 6

| Library | Vector | Library Description |
|---|---|---|
| BLADTUT02 | pINCY | Library was constructed using RNA isolated from bladder tumor tissue removed from |
| | | an 80-year-old Caucasian female during a radical cystectomy and lymph node |
| | | excision. Pathology indicated grade 3 invasive transitional cell carcinoma. Family |
| | | history included acute renal failure, osteoarthritis, and atherosclerosis. |
| BRABDIE02 | pINCY | This 5′ biased random primed library was constructed using RNA isolated from |
| | | diseased cerebellum tissue removed from the brain of a 57-year-old Caucasian male |
| | | who died from a cerebrovascular accident. Serologies were negative. Patient |
| | | history included Huntington's disease, emphysema, and tobacco abuse (3-4 packs per |
| | | day, for 40 years). |
| BRABDIR03 | pINCY | Library was constructed using RNA isolated from diseased cerebellum tissue removed |
| | | from the brain of a 57-year-old Caucasian male who died from a cerebrovascular |
| | | accident. Serologies were negative. Patient history included Huntington's |
| | | disease, emphysema, and tobacco abuse (3-4 packs per day for 40 years). |
| BRACNOK02 | PSPORT1 | This amplified and normalized library was constructed using RNA isolated from |
| | | posterior cingulate tissue removed from an 85-year-old Caucasian female who died |
| | | from myocardial infarction and retroperitoneal hemorrhage. Pathology indicated |
| | | atherosclerosis, moderate to severe, involving the circle of Willis, middle |
| | | cerebral, basilar and vertebral arteries; infarction, remote, left dentate |
| | | nucleus; and amyloid plaque deposition consistent with age. There was mild to |
| | | moderate leptomeningeal fibrosis, especially over the convexity of the frontal |
| | | lobe. There was mild generalized atrophy involving all lobes. The white matter was |
| | | mildly thinned. Cortical thickness in the temporal lobes, both maximal and |
| | | minimal, was slightly reduced. The substantia nigra pars compacta appeared mildly |
| | | depigmented. Patient history included COPD, hypertension, and recurrent deep |
| | | venous thrombosis. 6.4 million independent clones from this amplified library |
| | | were normalized in one round using conditions adapted Soares et al., PNAS (1994) |
| | | 91: 9228-9232 and Bonaldo et al., Genome Research 6 (1996): 791. |
| BRAHNOE01 | pINCY | Library was constructed RNA isolated from posterior hippocampus tissue removed |
| | | from a 45-year-old Caucasian female who died from a dissecting aortic aneurysm and |
| | | ischemic bowel disease. Pathology indicated mild arteriosclerosis involving the |
| | | cerebral cortical white matter and basal ganglia. Grossly, there was mild |
| | | meningeal fibrosis and mild focal atherosclerotic plaque in the middle cerebral |
| | | artery, as well as vertebral arteries bilaterally. Microscopically, the cerebral |
| | | hemispheres, brain stem and cerebellum reveal focal areas in the white matter that |
| | | contain blood vessels that were barrel-shaped, hyalinized, with hemosiderin-laden |
| | | macrophages in the Virchow-Robin space. In addition, there were scattered |
| | | neurofibrillary tangles within the basolateral nuclei of the amygdala. Patient |
| | | history included mild atheromatosis of aorta and coronary arteries, bowel and |
| | | liver infarct due to aneurysm, physiologic fatty liver associated with obesity, |
| | | mild diffuse emphysema, thrombosis of mesenteric and portal veins, cardiomegaly |
| | | due to hypertrophy of left ventricle, arterial hypertension, acute pulmonary |
| | | edema, splenomegaly, obesity (300 lb.), leiomyoma of uterus, sleep apnea, and iron |
| | | deficiency anemia. |
| BRAITDR03 | PCDNA2.1 | This random primed library was constructed using RNA isolated from allocortex, |
| | | cingulate posterior tissue removed from a 55-year-old Caucasian female who died |
| | | from cholangiocarcinoma. Pathology indicated mild meningeal fibrosis predominately |
| | | over the convexities, scattered axonal spheroids in the white matter of the |
| | | cingulate cortex and the thalamus, and a few scattered neurofibrillary tangles in |
| | | the entorhinal cortex and the periaqueductal gray region. Pathology for the |
| | | associated tumor tissue indicated well-differentiated cholangiocarcinoma of the |
| | | liver with residual or relapsed tumor. Patient history included |
| | | cholangiocarcinoma, post-operative Budd-Chiari syndrome, biliary ascites, |
| | | hydorthorax, dehydration, malnutrition, oliguria and acute renal failure. Previous |
| | | surgeries included cholecystectomy and resection of 85% of the liver. |
| BRATDIC01 | pINCY | This large size-fractionated library was constructed using RNA isolated from |
| | | diseased brain tissue removed from the left temporal lobe of a 27-year-old |
| | | Caucasian male during a brain lobectomy. Pathology for the left temporal lobe, |
| | | including the mesial temporal structures, indicated focal, marked pyramidal cell |
| | | loss and gliosis in hippocampal sector CA1, consistent with mesial temporal |
| | | sclerosis. The left frontal lobe showed a focal deep white matter lesion, |
| | | characterized by marked gliosis, calcifications, and hemosiderin-laden |
| | | macrophages, consistent with a remote perinatal injury. The frontal lobe tissue |
| | | also showed mild to moderate generalized gliosis, predominantly subpial and |
| | | subcortical, consistent with chronic seizure disorder. GFAP was positive for |
| | | astrocytes. The patient presented with intractable epilepsy, focal epilepsy, |
| | | hemiplegia, and an unspecified brain injury. Patient history included cerebral |
| | | palsy, abnormality of gait, depressive disorder, and tobacco abuse in remission. |
| | | Previous surgeries included tendon transfer. Patient medications included |
| | | minocycline hydrochloride, Tegretol, phenobarbital, vitamin C, Pepcid, and |
| | | Pevaryl. Family history included brain cancer in the father. |
| COLENOR03 | PCDNA2.1 | Library was constructed using RNA isolated from colon epithelium tissue removed |
| | | from a 13-year-old Caucasian female who died from a motor vehicle accident. |
| COLNNOT41 | pINCY | Library was constructed using RNA isolated from colon tissue removed from a 37- |
| | | year-old female during a partial gastrojejunectomy. Pathology indicated a portion |
| | | of stomach and jejunum with an intact anastomotic site. The stomach showed a mild |
| | | chronic gastritis without helicobacter pylori organisms. Normal appearing |
| | | submucosal and myenteric plexus ganglion cells were noted. The jejunum had no |
| | | significant abnormality. |
| CORPNOT02 | pINCY | Library was constructed using RNA isolated from diseased corpus callosum tissue |
| | | removed from the brain of a 74-year-old Caucasian male who died from Alzheimer's |
| | | disease. |
| DRGCNOT01 | pINCY | Library was constructed using RNA isolated from dorsal root ganglion tissue |
| | | removed from the cervical spine of a 32-year-old Caucasian male who died from |
| | | acute pulmonary edema and bronchopneumonia, bilateral pleural and pericardial |
| | | effusions, and malignant lymphoma (natural killer cell type). Patient history |
| | | included probable cytomegalovirus, infection, hepatic congestion and steatosis, |
| | | splenomegaly, hemorrhagic cystitis, thyroid hemorrhage, and Bell's palsy. |
| | | Surgeries included colonoscopy, large intestine biopsy, adenotonsillectomy, and |
| | | nasopharyngeal endoscopy and biopsy; treatment included radiation therapy. |
| ENDMUNE01 | pINCY | This 5′ biased random primed library was constructed using RNA isolated from |
| | | untreated umbilical artery endothelial cell tissue removed from a Caucasian male |
| | | (Clonetics) newborn. |
| ESOGTME01 | PSPORT1 | This 5′ biased random primed library was constructed using RNA isolated from |
| | | esophageal tissue removed from a 53-year-old Caucasian male during a partial |
| | | esophagectomy, proximal gastrectomy, and regional lymph node biopsy. Pathology |
| | | indicated no significant abnormality in the non-neoplastic esophagus. Pathology |
| | | for the matched tumor tissue indicated invasive grade 4 (of 4) adenocarcinoma, |
| | | forming a sessile mass situated in the lower esophagus, 2 cm from the |
| | | gastroesophageal junction and 7 cm from the proximal margin. The tumor invaded |
| | | through the muscularis propria into the adventitial soft tissue. Metastatic |
| | | carcinoma was identified in 2 of 5 paragastric lymph nodes with perinodal |
| | | extension. The patient presented with dysphagia. Patient history included |
| | | membranous nephritis, hyperlipidemia, benign hypertension, and anxiety state. |
| | | Previous surgeries included an adenotonsillectomy, appendectomy, and inguinal |
| | | hernia repair. The patient was not taking any medications. Family history included |
| | | atherosclerotic coronary artery disease, alcoholic cirrhosis, alcohol abuse, and |
| | | an abdominal aortic aneurysm rupture in the father; breast cancer in the mother; a |
| | | myocardial infarction and atherosclerotic coronary artery disease in the |
| | | sibling(s); and myocardial infarction and atherosclerotic coronary artery disease |
| | | in the grandparent(s). |
| HNT2AGT01 | PBLUESCRIPT | Library was constructed at Stratagene (STR937233), using RNA isolated from the |
| | | hNT2 cell line derived from a human teratocarcinoma that exhibited properties |
| | | characteristic of a committed neuronal precursor. Cells were treated with retinoic |
| | | acid for 5 weeks and with mitotic inhibitors for two weeks and allowed to mature |
| | | for an additional 4 weeks in conditioned medium. |
| ISLTNOT01 | pINCY | Library was constructed using RNA isolated from a pooled collection of pancreatic |
| | | islet cells. |
| KIDNNOT05 | PSPORT1 | Library was constructed using RNA isolated from the kidney tissue of a 2-day-old |
| | | Hispanic female, who died from cerebral anoxia. Family history included congenital |
| | | heart disease. |
| KIDNNOT09 | pINCY | Library was constructed using RNA isolated from the kidney tissue of a Caucasian |
| | | male fetus, who died at 23 weeks' gestation. |
| LEUKNOT02 | pINCY | Library was constructed using RNA isolated from white blood cells of a 45-year-old |
| | | female with blood type O+. The donor tested positive for cytomegalovirus (CMV). |
| LUNGNOT02 | PBLUESCRIPT | Library was constructed using RNA isolated from the lung tissue of a 47-year-old |
| | | Caucasian male, who died of a subarachnoid hemorrhage. |
| MUSCNOT07 | pINCY | Library was constructed using RNA isolated from muscle tissue removed from the |
| | | forearm of a 38-year-old Caucasian female during a soft tissue excision. Pathology |
| | | for the associated tumor tissue indicated intramuscular hemangioma. Family history |
| | | included breast cancer, benign hypertension, cerebrovascular disease, colon |
| | | cancer, and type II diabetes. |
| SINTNOR01 | PCDNA2.1 | This random primed library was constructed using RNA isolated from small intestine |
| | | tissue removed from a 31-year-old Caucasian female during Roux-en-Y gastric |
| | | bypass. Patient history included clinical obesity. |
| TESTNOT03 | PBLUESCRIPT | Library was constructed using RNA isolated from testicular tissue removed from a |
| | | 37-year-old Caucasian male, who died from liver disease. Patient history included |
| | | cirrhosis, jaundice, and liver failure. |

TABLE 7

| Program | Description | Reference | Parameter Threshold |
|---|---|---|---|
| ABI | A program that removes vector se- | Applied Biosystems, Foster City, CA. | |
| FACTURA | quences and masks ambiguous bases | | |
| | in nucleic acid sequences. | | |
| ABI/ | A Fast Data Finder useful in com- | Applied Biosystems, Foster City, CA; | Mismatch <50% |
| PARACEL | paring and annotating amino acid or | Paracel Inc., Pasadena, CA. | |
| FDF | nucleic acid sequences. | | |
| ABI | A program that assembles nucleic | Applied Biosystems, Foster City, CA. | |
| AutoAssembler | acid sequences. | | |
| BLAST | A Basic Local Alignment Search | Altschul, S. F. et al. (1990) J. Mol. Biol. | ESTs: Probability value = 1.0E−8 |
| | Tool useful in sequence similarity | 215:403-410; Altschul, S.F. et al. (1997) | or less |
| | search for amino acid and nucleic | Nucleic Acids Res. 25:3389-3402. | Full Length sequences: Probability |
| | acid sequences. BLAST includes five | | |
| | functions: blastp, blastn, blastx, | | value = 1.0E−10 or less |
| | tblastn, and tblastx. | | |
| FASTA | A Pearson and Lipman algorithm that | Pearson, W. R. and D. J. Lipman (1988) Proc. | ESTs: fasta E value = 1.06E−6 |
| | searches for similarity between a | Natl. Acad Sci. USA 85:2444-2448; Pearson, | Assembled ESTs: fasta Identity = |
| | query sequence and a group of se- | W. R. (1990) Methods Enzymol. 183:63-98; | 95% or greater and |
| | quences of the same type. FASTA | and Smith, T. F. and M. S. Waterman (1981) | Match length = 200 bases or greater; |
| | comprises as least five functions: | Adv. Appl. Math. 2:482-489. | fastx E value = 1.0E−8 or less |
| | fasta, tfasta, fastx, tfastx, and search. | Full Length sequences: | |
| | | fastx score = 100 or greater | |
| BLIMPS | A BLocks IMProved Searcher that | Henikoff, S. and J. G. Henikoff (1991) Nucleic | Probability value = 1.0E−3 or less |
| | matches a sequence against those in | Acids Res. 19:6565-6572; Henikoff, J. G. and | |
| | BLOCKS, PRINTS, DOMO, | S. Henikoff (1996) Methods Enzymol. | |
| | PRODOM, and PFAM databases to | 266:88-105; and Attwood, T. K. et al. (1997) J. | |
| | search for gene families, sequence | Chem. Inf. Comput. Sci. 37:417-424. | |
| | homology, and structural fingerprint | | |
| | regions. | | |
| HMMER | An algorithm for searching a query | Krogh, A. et al. (1994) J. Mol. Biol. | PFAM hits: Probability value = |
| | sequence against hidden Markov | 235:1501-1531; Sonnhammer, E.L.L. et al. | 1.0E−3 or less |
| | model (HMM)-based databases of | (1988) Nucleic Acids Res. 26:320-322; | Signal peptide hits: Score = 0 or |
| | protein family consensus sequences, | Durbin, R. et al. (1998) Our World View, in a | greater |
| | such as PFAM. | Nutshell, Cambridge Univ. Press, pp. 1-350. | |
| ProfileScan | An algorithm that searches for struc- | Gribskov, M. et al. (1988) CABIOS 4:61-66; | Normalized quality score ≧ GCG- |
| | tural and sequence motifs in protein | Gribskov, M. et al. (1989) Methods EnzymoL | specified “HIGH” value for that |
| | sequences that match sequence pat- | 183:146-159; Bairoch, A. et al. (1997) | particular Prosite motif. |
| | terns defined in Prosite. | Nucleic Acids Res. 25:217-221. | Generally, score = 1.4-2.1. |
| Phred | A base-calling algorithm that ex- | Ewing, B. et al. (1998) Genome Res. | |
| | amines automated sequencer traces | 8:175-185; Ewing, B. and P. Green | |
| | with high sensitivity and probability. | (1998) Genome Res. 8:186-194. | |
| Phrap | A Phils Revised Assembly Program | Smith, T. F. and M. S. Waterman (1981) Adv. | Score = 120 or greater; |
| | including SWAT and CrossMatch, | Appi Math. 2:482-489; Smith, T. F. and M. S. | Match length = 56 or greater |
| | programs based on efficient imple- | Waterman (1981) J. Mol. Biol. 147:195-197; | |
| | mentation of the Smith-Waterman | and Green, P., University of Washington, | |
| | algorithm, useful in searching se- | Seattle, WA. | |
| | quence homology and assembling | | |
| | DNA sequences. | | |
| Consed | A graphical tool for viewing and edit- | Gordon, D. et al. (1998) Genome Res. 8:195-202. | |
| | ing Phrap assemblies. | | |
| SPScan | A weight matrix analysis program | Nielson, H. et al. (1997) Protein Engineering | Score = 3.5 or greater |
| | that scans protein sequences for the | 10:1-6; Claverie, J. M. and S. Audic (1997) | |
| | presence of secretory signal peptides. | CABIOS 12:431∫439. | |
| TMAF | A program that uses weight matrices | Persson, B. and P. Argos (1994) J. Mol. Biol. | |
| | to delineate transmembrane segments | 237:182-192; Persson, B. and P. Argos (1996) | |
| | on protein sequences and determine | Protein Sci. 5:363-371. | |
| | orientation. | | |
| TMHMMER | A program that uses a hidden Markov | Sonnhaxnmer, E. L. et at. (1998) Proc. Sixth Intl. | |
| | model (HMM) to delineate trans- | Conf. on Intelligent Systems for Mol. Biol., | |
| | membrane segments on protein se- | Glasgow et al., eds., The Am. Assoc. for Artificial | |
| | quences and determine orientation. | Intelligence Press, Menlo Park, CA, pp. 175-182. | |
| Motifs | A program that searches amino acid | Bairoch, A. et al. (1997) Nucleic Acids Res. 25:217-221; | |
| | sequences for pattems that matched | Wisconsin Package Program Manual, version 9, page | |
| | those defined in Prosite. | M51-59, Genetics Computer Group, Madison, WI. | |

What is claimed is:

1. An isolated polypeptide selected from the group consisting of:
a) a polypeptide comprising an amino acid sequence selected from the group consisting of SEQ ID NO: 1-26,
b) a polypeptide comprising a naturally occurring amino acid sequence at least 90% identical to an amino acid sequence selected from the group consisting of SEQ ID NO: 1-26,
c) a biologically active fragment of a polypeptide having an amino acid sequence selected from the group consisting of SEQ ID NO: 1-26, and
d) an immunogenic fragment of a polypeptide having an amino acid sequence selected from the group consisting of SEQ ID NO: 1-26.

2. An isolated polypeptide of claim 1 selected from the group consisting of SEQ ID NO: 1-26.

3. An isolated polynucleotide encoding a polypeptide of claim 1.

4. An isolated polynucleotide encoding a polypeptide of claim 2.

5. An isolated polynucleotide of claim 4 selected from the group consisting of SEQ ID NO: 27-52.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method for producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. An isolated antibody which specifically binds to a polypeptide of claim 1.

11. An isolated polynucleotide selected from the group consisting of:
a) a polynucleotide comprising a polynucleotide sequence selected from the group consisting of SEQ ID NO: 27-52,
b) a polynucleotide comprising a naturally occurring polynucleotide sequence at least 90% identical to a polynucleotide sequence selected from the group consisting of SEQ ID NO: 27-52,
c) a polynucleotide complementary to a polynucleotide of a),
d) a polynucleotide complementary to a polynucleotide of b), and
e) an RNA equivalent of a)-d).

12. An isolated polynucleotide comprising at least 60 contiguous nucleotides of a polynucleotide of claim 11.

13. A method for detecting a target polynucleotide in a sample, said target polynucleotide having a sequence of a polynucleotide of claim 11, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

14. A method of claim 13, wherein the probe comprises at least 60 contiguous nucleotides.

15. A method for detecting a target polynucleotide in a sample, said target polynucleotide having a sequence of a polynucleotide of claim 11, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

16. A composition comprising a polypeptide of claim 1 and a pharmaceutically acceptable excipient.

17. A composition of claim 16, wherein the polypeptide has an amino acid sequence selected from the group consisting of SEQ ID NO: 1-26.

18. A method for treating a disease or condition associated with decreased expression of functional PKIN, comprising administering to a patient in need of such treatment the composition of claim 16.

19. A method for screening a compound for effectiveness as an agonist of a polypeptide of claim 1, the method comprising:
a) exposing a sample comprising a polypeptide of claim 1 to a compound, and
b) detecting agonist activity in the sample.

20. A composition comprising an agonist compound identified by a method of claim 19 and a pharmaceutically acceptable excipient

21. A method for treating a disease or condition associated with decreased expression of functional PKIN, comprising administering to a patient in need of such treatment a composition of claim 20.

22. A method for screening a compound for effectiveness as an antagonist of a polypeptide of claim 1, the method comprising:
a) exposing a sample comprising a polypeptide of claim 1 to a compound, and
b) detecting antagonist activity in the sample.

23. A composition comprising an antagonist compound identified by a method of claim 22 and a pharmaceutically acceptable excipient.

24. A method for treating a disease or condition associated with overexpression of functional PKIN, comprising administering to a patient in need of such treatment a composition of claim 23.

25. A method of screening for a compound that specifically binds to the polypeptide of claim 1, said method comprising the steps of:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

26. A method of screening for a compound that modulates the activity of the polypeptide of claim 1, said method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

27. A method for screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

28. A method for assessing toxicity of a test compound, said method comprising:
a) treating a biological sample containing nucleic acids with the test compound;
b) hybridizing the nucleic acids of the treated biological sample with a probe comprising at least 20 contiguous nucleotides of a polynucleotide of claim 11 under conditions whereby a specific hybridization complex is formed between said probe and a target polynucleotide in the biological sample, said target polynucleotide comprising a polynucleotide sequence of a polynucleotide of claim 11 or fragment thereof;
c) quantifying the amount of hybridization complex; and
d) comparing the amount of hybridization complex in the treated biological sample with the amount of hybridization complex in an untreated biological sample, wherein a difference in the amount of hybridization complex in the treated biological sample is indicative of toxicity of the test compound.

29. A diagnostic test for a condition or disease associated with the expression of PKIN in a biological sample comprising the steps of:
a) combining the biological sample with an antibody of claim 10, under conditions suitable for the antibody to bind the polypeptide and form an antibody:polypeptide complex; and
b) detecting the complex, wherein the presence of the complex correlates with the presence of the polypeptide in the biological sample.

30. The antibody of claim 10, wherein the antibody is:
a) a chimeric antibody,
b) a single chain antibody,
c) a Fab fragment,
d) a F(ab′)₂ fragment, or
e) a humanized antibody.

31. A composition comprising an antibody of claim 10 and an acceptable excipient.

32. A method of diagnosing a condition or disease associated with the expression of PKIN in a subject, comprising administering to said subject an effective amount of the composition of claim 31.

33. A composition of claim 31, wherein the antibody is labeled.

34. A method of diagnosing a condition or disease associated with the expression of PKIN in a subject, comprising administering to said subject an effective amount of the composition of claim 33.

35. A method of preparing a polyclonal antibody with the specificity of the antibody of claim 10 comprising:
a) immunizing an animal with a polypeptide having an amino acid sequence selected from the group consisting of SEQ ID NO: 1-26, or an immunogenic fragment thereof, under conditions to elicit an antibody response;
b) isolating antibodies from said animal; and
c) screening the isolated antibodies with the polypeptide, thereby identifying a polyclonal antibody which binds specifically to a polypeptide having an amino acid sequence selected from the group consisting of SEQ ID NO: 1-26.

36. An antibody produced by a method of claim 35.

37. A composition comprising the antibody of claim 36 and a suitable carrier.

38. A method of making a monoclonal antibody with the specificity of the antibody of claim 10 comprising:
a) immunizing an animal with a polypeptide having an amino acid sequence selected from the group consisting of SEQ ID NO: 1-26, or an immunogenic fragment thereof, under conditions to elicit an antibody response;
b) isolating antibody producing cells from the animal;
c) fusing the antibody producing cells with immortalized cells to form monoclonal antibody-producing hybridoma cells;
d) culturing the hybridoma cells; and
e) isolating from the culture monoclonal antibody which binds specifically to a polypeptide having an amino acid sequence selected from the group consisting of SEQ ID NO: 1-26.

39. A monoclonal antibody produced by a method of claim 38.

40. A composition comprising the antibody of claim 39 and a suitable carrier.

41. The antibody of claim 10, wherein the antibody is produced by screening a Fab expression library.

42. The antibody of claim 10, wherein the antibody is produced by screening a recombinant immunoglobulin library.

43. A method for detecting a polypeptide having an amino acid sequence selected from the group consisting of SEQ ID NO: 1-26 in a sample, comprising the steps of:
a) incubating the antibody of claim 10 with a sample under conditions to allow specific binding of the antibody and the polypeptide; and
b) detecting specific binding, wherein specific binding indicates the presence of a polypeptide having an amino acid sequence selected from the group consisting of SEQ ID NO: 1-26 in the sample.

44. A method of purifying a polypeptide having an amino acid sequence selected from the group consisting of SEQ ID NO: 1-26 from a sample, the method comprising:
a) incubating the antibody of claim 10 with a sample under conditions to allow specific binding of the antibody and the polypeptide; and
b) separating the antibody from the sample and obtaining the purified polypeptide having an amino acid sequence selected from the group consisting of SEQ ID NO: 1-26.

45. A polypeptide of claim 1, comprising the amino acid sequence of SEQ ID NO: 1.

46. A polypeptide of claim 1, comprising the amino acid sequence of SEQ ID NO: 2.

47. A polypeptide of claim 1, comprising the amino acid sequence of SEQ ID NO: 3.

48. A polypeptide of claim 1, comprising the amino acid sequence of SEQ ID NO: 4.

49. A polypeptide of claim 1, comprising the amino acid sequence of SEQ ID NO: 5.

50. A polypeptide of claim 1, comprising the amino acid sequence of SEQ ID NO: 6.

51. A polypeptide of claim 1, comprising the amino acid sequence of SEQ ID NO: 7.

52. A polypeptide of claim 1, comprising the amino acid sequence of SEQ ID NO: 8.

53. A polypeptide of claim 1, comprising the amino acid sequence of SEQ ID NO: 9.

54. A polypeptide of claim 1, comprising the amino acid sequence of SEQ ID NO: 10.

55. A polypeptide of claim 1, comprising the amino acid sequence of SEQ ID NO: 11.

56. A polypeptide of claim 1, comprising the amino acid sequence of SEQ ID NO: 12.

57. A polypeptide of claim 1, comprising the amino acid sequence of SEQ ID NO: 13.

58. A polypeptide of claim 1, comprising the amino acid sequence of SEQ ID NO: 14.

59. A polypeptide of claim 1, comprising the amino acid sequence of SEQ ID NO: 15.

60. A polypeptide of claim 1, comprising the amino acid sequence of SEQ ID NO: 16.

61. A polypeptide of claim 1, comprising the amino acid sequence of SEQ ID NO: 17.

62. A polypeptide of claim 1, comprising the amino acid sequence of SEQ ID NO: 18.

63. A polypeptide of claim 1, comprising the amino acid sequence of SEQ ID NO: 19.

64. A polypeptide of claim 1, comprising the amino acid sequence of SEQ ID NO: 20.

65. A polypeptide of claim 1, comprising the amino acid sequence of SEQ ID NO: 21.

66. A polypeptide of claim 1, comprising the amino acid sequence of SEQ ID NO: 22.

67. A polypeptide of claim 1, comprising the amino acid sequence of SEQ ID NO: 23.

68. A polypeptide of claim 1, comprising the amino acid sequence of SEQ ID NO: 24.

69. A polypeptide of claim 1, comprising the amino acid sequence of SEQ ID NO: 25.

70. A polypeptide of claim 1, comprising the amino acid sequence of SEQ ID NO: 26.

71. A polynucleotide of claim 11, comprising the polynucleotide sequence of SEQ ID NO: 27.

72. A polynucleotide of claim 11, comprising the polynucleotide sequence of SEQ ID NO: 28.

73. A polynucleotide of claim 11, comprising the polynucleotide sequence of SEQ ID NO: 29.

74. A polynucleotide of claim 11, comprising the polynucleotide sequence of SEQ ID NO: 30.

75. A polynucleotide of claim 11, comprising the polynucleotide sequence of SEQ ID NO: 31.

76. A polynucleotide of claim 11, comprising the polynucleotide sequence of SEQ ID NO: 32.

77. A polynucleotide of claim 11, comprising the polynucleotide sequence of SEQ ID NO: 33.

78. A polynucleotide of claim 11, comprising the polynucleotide sequence of SEQ ID NO: 34.

79. A polynucleotide of claim 11, comprising the polynucleotide sequence of SEQ ID NO: 35.

80. A polynucleotide of claim 11, comprising the polynucleotide sequence of SEQ ID NO: 36.

81. A polynucleotide of claim 11, comprising the polynucleotide sequence of SEQ ID NO: 37.

82. A polynucleotide of claim 11, comprising the polynucleotide sequence of SEQ ID NO: 38.

83. A polynucleotide of claim 11, comprising the polynucleotide sequence of SEQ ID NO: 39.

84. A polynucleotide of claim 11, comprising the polynucleotide sequence of SEQ ID NO: 40.

85. A polynucleotide of claim 11, comprising the polynucleotide sequence of SEQ ID NO: 41.

86. A polynucleotide of claim 11, comprising the polynucleotide sequence of SEQ ID NO: 42.

87. A polynucleotide of claim 11, comprising the polynucleotide sequence of SEQ ID NO: 43.

88. A polynucleotide of claim 11, comprising the polynucleotide sequence of SEQ ID NO: 44.

89. A polynucleotide of claim 11, comprising the polynucleotide sequence of SEQ ID NO: 45.

90. A polynucleotide of claim 11, comprising the polynucleotide sequence of SEQ-ID NO: 46.

91. A polynucleotide of claim 11, comprising the polynucleotide sequence of SEQ ID NO: 47.

92. A polynucleotide of claim 11, comprising the polynucleotide sequence of SEQ ID NO: 48.

93. A polynucleotide of claim 11, comprising the polynucleotide sequence of SEQ ID NO: 49.

94. A polynucleotide of claim 11, comprising the polynucleotide sequence of SEQ ID NO: 50.

95. A polynucleotide of claim 11, comprising the polynucleotide sequence of SEQ ID NO: 51.

96. A polynucleotide of claim 11, comprising the polynucleotide sequence of SEQ ID NO: 52.

97. A method of claim 9, wherein the polypeptide has the sequence of SEQ ID NO: 1.

98. A method of claim 9, wherein the polypeptide has the sequence of SEQ ID NO: 2.

99. A method of claim 9, wherein the polypeptide has the sequence of SEQ ID NO: 3.

100. A method of claim 9, wherein the polypeptide has the sequence of SEQ ID NO: 4.

101. A method of claim 9, wherein the polypeptide has the sequence of SEQ ID NO: 5.

102. A method of claim 9, wherein the polypeptide has the sequence of SEQ ID NO: 6.

103. A method of claim 9, wherein the polypeptide has the sequence of SEQ ID NO: 7.

104. A method of claim 9, wherein the polypeptide has the sequence of SEQ ID NO: 8.

105. A method of claim 9, wherein the polypeptide has the sequence of SEQ ID NO: 9.

106. A method of claim 9, wherein the polypeptide has the sequence of SEQ ID NO: 10.

107. A method of claim 9, wherein the polypeptide has the sequence of SEQ ID NO: 11.

108. A method of claim 9, wherein the polypeptide has the sequence of SEQ ID NO: 12.

109. A method of claim 9, wherein the polypeptide has the sequence of SEQ ID NO: 13.

110. A method of claim 9, wherein the polypeptide has the sequence of SEQ ID NO: 14.

111. A method of claim 9, wherein the polypeptide has the sequence of SEQ ID NO: 15.

112. A method of claim 9, wherein the polypeptide has the sequence of SEQ ID NO: 16.

113. A method of claim 9, wherein the polypeptide has the sequence of SEQ ID NO: 17.

114. A method of claim 9, wherein the polypeptide has the sequence of SEQ ID NO: 18.

115. A method of claim 9, wherein the polypeptide has the sequence of SEQ ID NO: 19.

116. A method of claim 9, wherein the polypeptide has the sequence of SEQ ID NO: 20.

117. A method of claim 9, wherein the polypeptide has the sequence of SEQ ID NO: 21.

118. A method of claim 9, wherein the polypeptide has the sequence of SEQ ID NO: 22.

119. A method of claim 9, wherein the polypeptide has the sequence of SEQ ID NO: 23.

120. A method of claim 9, wherein the polypeptide has the sequence of SEQ ID NO: 24.

121. A method of claim 9, wherein the polypeptide has the sequence of SEQ ID NO: 25.

122. A method of claim 9, wherein the polypeptide has the sequence of SEQ ID NO: 26.

123. A microarray wherein at least one element of the microarray is a polynucleotide of claim 12.

124. A method for generating a transcript image of a sample which contains polynucleotides, the method comprising the steps of:
a) labeling the polynucleotides of the sample,
b) contacting the elements of the microarray of claim 123 with the labeled polynucleotides of the sample under conditions suitable for the formation of a hybridization complex, and
c) quantifying the expression of the polynucleotides in the sample.

125. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, said target polynucleotide having a sequence of claim 11.

126. An array of claim 125, wherein said first oligonucleotide or polynucleotide sequence is completely complementary to at least 30 contiguous nucleotides of said target polynucleotide.

127. An array of claim 125, wherein said first oligonucleotide or polynucleotide sequence is completely complementary to at least 60 contiguous nucleotides of said target polynucleotide.

128. An array of claim 125, which is a microarray.

129. An array of claim 125, further comprising said target polynucleotide hybridized to said first oligonucleotide or polynucleotide.

130. An array of claim 125, wherein a linker joins at least one of said nucleotide molecules to said solid substrate.

131. An array of claim 125, wherein each distinct physical location on the substrate contains multiple nucleotide molecules having the same sequence, and each distinct physical location on the substrate contains nucleotide molecules having a sequence which differs from the sequence of nucleotide molecules at another physical location on the substrate.


SEQUENCE LISTING (52 sequences)

SEQ ID NO: 1 - protein, 273 aa, Homo sapiens
  misc_feature: Incyte ID No 2011384CD1
MSGDKLLSELGYKLGRTIGEGSYSKVKVATSKKYKGTVAIKVVDRRRAPPDFVNKFLPRELSILRGVRHPHIVHVFEFIEVCNGKLYIVMEAAATDLLQAVQRNGRIPGVQARDLFAQIAGAVRYLHDHHLVHRDLKCENVLLSPDERRVKLTDFGFGRQAHGYPDLSTTYCGSAAYASPEVLLGIPYDPKKYDVWSMGVVLYVMVTGCMPFDDSDIAGLPRRQKRGVLYPEGLELSERCKALIAELLQFSPSARPSAGQVARNCWLRAGDSG

SEQ ID NO: 2 - protein, 329 aa, Homo sapiens
  misc_feature: Incyte ID No 2004888CD1
MLTSLAQKWFPELPLLHPEIGLLKYMNSGGLLTMSLERDLLDAEPMKELSSKRPLVRSEVNGQIILLKGYSVDVDTEAKVIERAATYHRAWREAEGDSGLLPLIFLFLCKSDPMAYLMVPYYPRANLNAVQANMPLNSEETLKVMKGVAQGLHTLHKADIIHGSLHQNNVFALNREQGIVGDFDFTKSVSQRASVNMMVGDLSLMSPELKMGKPASPGSDLYAYGCLLLWLSVQNQEFEINKDGIPKVDQFHLDDKVKSLLCSLICYRSSMTAEQVLNAECFLMPKEQSVPNPEKDTEYTLYKKEEEIKTENLDKCMEKTRNGEANFDC

SEQ ID NO: 3 - protein, 938 aa, Homo sapiens
  misc_feature: Incyte ID No 2258952CD1
MMSDTSTFPNHPSSPAASPSGGRGVMASPAWDRSKGWSQTPQRADFVSTPLQVHTLRPENLLLVSTLDGSLHALSKQTGDLKWTLRDDPVIEGPMYVTEMAFLSDPADGSLYILGTQKQQGLMKLPFTIPELVHASPCRSSDGVFYTGRKQDAWFVVDPESGETQMTLTTEGPSTPRLYIGRTQYTVTMHDPRAPALRWNTTYRRYSAPPMDGSPGKYMSHLASCGMGLLLTVDPGSGTVLWTQDLGVPVMGVYTWHQDGLRQLPHLTLARDTLHFLALRWGHIRLPASGPRDTATLFSTLDTQLLMTLYVGKDETGFYVSKALVHTGVALVPRGLTLAPADGPTTDEVTLQVSGEREGSPSTAVRYPSGSVALPSQWLLIGHHELPPVLHTTMLRVHPTLGSGTAETRPPENTQAPAFFLELLSLSREKLWDSELHPEEKTPDSYLGLGPQDLLAASLTAVLLGGWILFVMRQQQETPLAPADFAHISQDAQSLHSGASRRSQKRLQSPSPESPPSSPPAEQLTVVGKISFNPKDVLGRGAGGTFVFRGQFEGRAVAVKRLLRECFGLVRREVQLLQESDRHPNVLRYFCTERGPQFHYIALELCRASLQEYVENPDLDRGGLEPEVVLQQLMSGLAHLHSLHIVHRDLKPGNILITGPDSQGLGRVVLSDFGLCKKLPAGRCSFSLHSGIPGTEGWMAPELLQLLPPDSPTSAVDIFSAGCVFYYVLSGGSHPFGDSLYRQANILTGAPCLAHLEEEVHDKVVARDLVGAMLSPLPQPRPSAPQVLAHPFFWSRAKQLQFFQDVSDWLEKESEQEPLVRALEAGGCAVVRDNWHEHISMPLQTDLRKFRSYKGTSVRDLLRAVRNKKHHYRELPVEVRQALGQVPDGFVQYFTNRFPRLLLHTHRAMRSCASESLFLPYYPPDSEARRPCPGATGR

SEQ ID NO: 4 - protein, 795 aa, Homo sapiens
  misc_feature: Incyte ID No 7473244CD1
MSARTPLPTVNERDTENHTSVDGYTEPHIQPTKSSSRQNIPRCRNSITSATDEQPHIGNYRLQKTIGKGNFAKVKLARHVLTGREVAVKIIDKTQLNPTSLQKLFREVRIMKILNHPNIVKLFEVIETEKTLYLVMEYASGGEVFDYLVAHGRMKEKEARAKFRQIVSAVQYCHQKYIVHRDLKAENLLLDGDMNIKIADFGFSNEFTVGNKLDTFCGSPPYAAPELFQGKKYDGPEVDVWSLGVILYTLVSGSLPFDGQNLKELRERVLRGKYRIPFYMSTDCENLLKKLLVLNPIKRGSLEQIMKDRWMNVGHEEEELKPYTEPDPDFNDTKRIDIMVTMGFARDEINDALINQKYDEVMATYILLGRKPPEFEGGESLSSGNLCQRSRPSSDLNNSTLQSPAHLKVQRSISANQKQRRFSDHAGPSIPPAVSYTKRPQANSVESEQKEEWDKDVARKLGSTTVGSKSEMTASPLVGPERKKSSTIPSNNVYSGGSMARRNTYVCERTTDRYVALQNGKDSSLTEMSVSSISSAGSSVASAVPSARPRHQKSMSTSGHPIKVTLPTIKDGSEAYRPGTTQRVPAASPSAHSISTATPDRTRFPRGSSSRSTFHGEQLRERRSVAYNGPPASPSHETGAFAHARRGTSTGIISKITSKFVRRDPSEGEASGRTDTSRSTSGEPKERDKEEGKDSKPRSLRFTWSMKTTSSMDPNDMMREIRKVLDANNCDYEQKERFLLFCVHGDARQDSLVQWEMEVCKLPRLSLNGVRFKRISGTSIAFKNIASKIANELKL

SEQ ID NO: 5 - protein, 656 aa, Homo sapiens
  misc_feature: Incyte ID No 1242491CD1
MMSWNLNKLQSFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVTVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 6 - protein, 596 aa, Homo sapiens
  misc_feature: Incyte ID No 2634875CD1
MATENGAVELGIQNPSTDKAPKGPTGERPLAAGKDPGPPDPKKAPDPPTLKKDAKAPASEKGDGTLAQPSTSSQGPKGEGDRGGGPAEGSAGPPAALPQQTATPETSVKKPKAEQGASGSQDPGKPRVGKKAAEGQAAARRGSPAFLHSPSCPAIISSSEKLLAKKPPSEASELTFEGVPMTHSPTDPRPAKAEEGKNILAESQKEVGEKTPGQAGQAKMQGDTSRGIEFQAVPSEKSEVGQALCLTAREEDCFQILDDCPPPPAPFPHRMVELRTGNVSSEFSMNSKEALGGGKFGAVCTCMEKATGLKLAAKVIKKQTPKDKEMVLLEIEVMNQLNHRNLIQLYAAIETPHEIVLFMEYIEGGELFERIVDEDYHLTEVDTMVFVRQICDGILFSVLERVLHLDLKPENILCVNTTGHLVKIIDFGLARRYNPNEKLKVNFGTPEFLSPEVVKGDQISDKTDMWSMGVITYMLLSGLSPFLGDDDTETLNNVLSGNWYFDEETFEAVSDEAKDFVSNLIVKDQRARMNAAQCLAHPWLNNLAEKAKRCNRRLKSQILLKKYLMKRRWKKNFIAVSAANRFKKISSSGALMALGV

SEQ ID NO: 7 - protein, 497 aa, Homo sapiens
  misc_feature: Incyte ID No 3951059CD1
MLKFKYGARNPLDAGAAEPIASRASRLNLFFQGKPPFMTQQQMSPLSREGILDALFVLFEECSQPALMKIKHVSNFVRKYSDTIAELQELQPSAKDFEVRSLVGCGHFAEVQVVREKATGDIYAMKVMKKKALLAQEQVSFFEEERNILSRSTSPWIPQLQYAFQDKNHLYLVMEYQPGGDLLSLLNRYEDQLDENLIQFYLAELILAVHSVHLMGYVHRDIKPENILVDRTGHIKLVDFGSAAKMNSNKMVNAKLPIGTPDYMAPEVLTVMNGDGKGTYRLDCDWWSVGVIAYEMIYGRSPFAEGTSARTFNNIMNFQRFLKFPDDPKVSSDFLDLIQSLLCGQKERLKFEGLCCHPFFSKIDWNNIRNSPPPFVPTLKSDDDTSNFDEPEKNSWVSSSPCQLSPSGFSGEELPFVGFSYSKALGILGRSESVVSGLDSPAKTSSMEKKLLIKSKELQDSQDKCHKVFISAAGLLPCSRILPSVYAKGSARGRCWL

SEQ ID NO: 8 - protein, 1171 aa, Homo sapiens
  misc_feature: Incyte ID No 7395890CD1
MAPVYEGMASHVQVFSPHTLQSSAFCSVKKLKIEPSSNWDMTGYGSHSKVYSQSKNIPLSQPATTTVSTSLPVPNPSLPYEQTIVFPGSTGHIVVTSASSTSVTGQVLGGPHNLMRRSTVSLLDTYQKCGLKRKSEEIENTSSVQIIEEHPPMIQNNASGATVATATTSTATSKNSGSNSEGDYQLVQHEVLCSMTNTYEVLEFLGRGTFGQVVKCWKRGTNEIVAIKILKNHPSYARQGQIEVSILARLSTESADDYNFVRAYECFQHKNHTCLVFEMLEQNLYDFLKQNKFSPLPLKYIRPVLQQVATALMKLKSLGLIHADLKPENIMLVDPSRQPYRVKVIDFGSASHVSKAVCSTYLQSRYYRAPEIILGLPFCEAIDMWSLGCVIAELFLGWPLYPGASEYDQIRYISQTQGLPAEYLLSAGTKTTRFFNRDTDSPYPLWRLKTPDDHEAETGIKSKEARKYIFNCLDDMAQVNMTTDLEGSDMLVEKADRREFIDLLKKMLTIDADKRITPIETLNHPFVTMTHLLDFPHSTHVKSCFQNMEICKRRVNMYDTVNQSKTPFITHVAPSTSTNLTMTFNNQLTTVHNQPSAASMAAVAQRSMPLQTGTAQICARPDPFQQALIVCPPGFQGLQASPSKHAGYSVRMENAVPIVTQAPGAQPLQIQPGLLAQQAWPSGTQQILLPPAWQQLTGVATHTSVQHATVIPETMAGTQQLADWRNTHAHGSHYNPIMQQPALLTGHVTLPAAQPLNVGVAHVMRQQPTSTTSSRKSKQHQSSVRNVSTCEVSSSQAISSPQRSKRVKENTPPRCAMVHSSPACSTSVTCGWGDVASSTTRERQRQTIVIPDTPSPTVSVITISSDTDEEEEQKHAPTSTVSKQRKNVISCVTVHDSPYSDSSSNTSPYSVQQRAGHNNANAFDTKGSLENHCTGNPRTIIVPPLKTQASEVLVECDSLVPVNTSHHSSSYKSKSSSNVTSTSGHSSGSSSGAITYRQQRPGPHFQQQQPLNLSQAQQHITTDRTGSHRRQQAYITPTMAQAPYSFPHNSPSHGTVHPHLAAAAAAAHLPTQPHLYTYTAPAALGSTGTVAHLVASQGSARHTVQHTAYPASIVHQVPVSMGPRVLPSPTIHPSQYPAQFAHQTYISASPASTVYTGYPLSPAKVNQYPYI

SEQ ID NO: 9 - protein, 470 aa, Homo sapiens
  misc_feature: Incyte ID No 7475546CD1
MAGPGWGPPRLDGFILTERLGSGTYATVYKAYAKKDTREVVAIKCVAKKSLNKASVENLLTEIEILKGIRHPHIVQLKDFQWDSDNIYLIMEFCAGGDLSRFIHTRRILPEKVARVFMQQLASALQFLHERNISHLDLKPQNILLSSLEKPHLKLADFGFAQHMSPWDEKHVLRGSPLYMAPEMVCQRQYDARVDLWSMGVILYEALFGQPPFASRSFSELEEKIRSNRVIELPLRPLLSRDCRDLLQRLLERDPSRRISFQDFFAHPWVDLEHMPSGESLGRATALVVQAVKKDQEGDSAAALSLYCKALDFFVPALHYEVDAQRKEAIKAKVGQYVSRAEELKAIVSSSNQALLRQGTSARDLLREMARDKPRLLAALEVASAAMAKEEAAGGEQDALDLYQHSLGELLLLLAAEPPGRRRELLHTEVQNLMARAEYLKEQMRESRWEADTLDKEGLSESVRSSCTLQ

SEQ ID NO: 10 - protein, 422 aa, Homo sapiens
  misc_feature: Incyte ID No 7477076CD1
MDHPSREKDERQRTTKPMAQRSAHCSRPSGSSSSSGVLMVGPNFRVGKKIGCGNFGELRLGKNLYTNEYVAIKLEPIKSRAPQLHLEYRFYKQLGSAGEGLPQVYYFGPCGKYNAMVLELLGPSLEDLFDLCDRTFTLKTVLMIAIQLLSRMEYVHSKNLIYRDVKPENFLIGRQGNKKEHVIHIIDFGLAKEYIDPETKKHIPYREHKSLTGTARYMSINTHLGKEQSRRDDLEALGHMFMYFLRGSLPWQGLKADTLKERYQKIGDTKRNTPIEALCENFPEEMATYLRYVRRLDFFEKPDYEYLRTLFTDLFEKKGYTFDYAYDWVGRPIPTPVGSVHVDSGASAITRESHTHRDRPSQQQPLRNQVVSSTNGELNVDDPTGAHSNAPITAHAEVEVVEEAKCCCFFKRKRKKTAQRHK

SEQ ID NO: 11 - protein, 240 aa, Homo sapiens
  misc_feature: Incyte ID No 1874092CD1
MPVSKCPKKSESLWKGWDRKAQRNGLRSQVYAVNGDYYVGEWKDNVKHGKGTQVWKKKGAIYEGDWKFGKRDGYGTLSLPDQQTGKCRRVYSGWWKGDKKSGYGIQFFGPKEYYEGDWCGSQRSGWGRMYYSNGDIYEGQWENDKPNGEGMLRLKNGNRYEGCWERGMKNGAGRFFHLDHGQLFEGFWVDNMAKCGTMIDFGRDEAPEPTQFPIPEVKILDPDGVLAEALAMFRKTEEGD

SEQ ID NO: 12 - protein, 594 aa, Homo sapiens
  misc_feature: Incyte ID No 4841542CD1
MKKQAVKRHHHKHNLRHRYEFLETLGKGTYGKVKKARESSGRLVAIKSIRKDKIKDEQDLMHIRREIEIMSSLNHPHIIAIHEVFENSSKIVIVMEYASRGDLYDYISERQQLSEREARHFFRQIVSAVHYCHQNRVVHRDLKLENILLGANGNIKIADFGLSNLYHQGKFLQTFCGSPLYASPEIVNGKPYTGPEVDSWSLGVLLYILVHGTMPFDGHDHKILVKQISNGAYREPPKPSDACGLIRWLLMVNPTRRATLEDVASHWWVNWGYATRVGEQEAPHEGGHPGSDSARASMADWLRRSSRPLLENGAKVCSFFKQHAPGGGSTTPGLERQHSLKKSRKENDMAQSLHSDTADDTAHRPGKSNLKLPKGILKKKVSASAEGVQEDPPELSPIPASPGQAAPLLPKKGILKKPRQRESGYYSSPEPSESGELLDAGDVFVSGDPKEQKPPQASGLLLHRKGILKLNGKFSQTALELAAPTTFGSLDELAPPRPLARASRPSGAVSEDSILSSESFDQLDLPERLPEPPLRGCVSVDNLTGLEEPPSEGPGSCLRRWRQDPLGDSCFSLTDCQEVTATYRQALRVCSKLT

SEQ ID NO: 13 - protein, 473 aa, Homo sapiens
  misc_feature: Incyte ID No 7472695CD1
MSQTSSIGSAESLISLERKKEKNINRDITSRKDLPSRTSNVERKASQQQWGRGNFTEGKVPHIRIENGAAIEEIYTFGRILGKGSFGIVIEATDKETETKWAIKKVNKEKAGSSAVKLLEREVNILKSVKHEHIIHLEQVFETPKKMYLVMELCEDGELKEILDRKGHFSENETRWIIQSLASAIAYLHNNDIVHRDLKLENIMVKSSLIDDNNEINLNIKVTDFGLAVKKQSRSEAMLQATCGTPIYMAPEVISAHDYSQQCDIWSIGVVMYMLLRGEPPFLASSEEKLFELIRKGELHFENAVWNSISDCAKSVLKQLMKVDPAHRITAKELLDNQWLTGNKLSSVRPTNVLEMMKEWKNNPESVEENTTEEKNKPSTEEKLKSYQPWGNVPDANYTSDEEEEKQSTAYEKQFPATSKDNFDMCSSSFTSSKLLPAEIKGEMEKTPVTPSQGTATKYPAKSGALSRTKKKL

SEQ ID NO: 14 - protein, 947 aa, Homo sapiens
  misc_feature: Incyte ID No 7477966CD1
MMSDTSTFPNHPSSPAASPSGGRGVMASPAWDRSKGWSQTPQRADFVSTPLQVHTLRPENLLLVSTLDGSLHALSKQTGDLKWTLRDDPVIEGPMYVTEMAFLSDPADGSLYILGTQKQQGLMKLPFTIPELVHASPCRSSDGVFYTGRKQDAWFVVDPESGETQMTLTTEGPSTPRLYIGRTQYTVTMHDPRAPALRWNTTYRRYSAPPMDGSPGKYMSHLASCGMGLLLTVDPGSGTVLWTQDLGVPVMGVYTWHQDGLRQLPHLTLARDTLHFLALRWGHIRLPASGPRDTATLFSTLDTQLLMTLYVGKDETGFYVSKALVHTGVALVPRGLTLAPADGPTTDEVTLQVSGEREGSPSTAVRYPSGSVALPSQWLLIGHHELPPVLHTTMLRVHPTLGSGTAETRPPENTQAPAFFLELLSLSREKLWDSELHPEEKTPDSYLGLGPQDLLAASLTAVLLGGWILFVMRQQQPQVVEKQQETPLAPADFAHISQDAQSLHSGASRRSQKRLQSPSKQAQPLDDPEAEQLTVVGKISFNPKDVLGRGAGGTFVFRGQFEGRAVAVKRLLRECFGLVRREVQLLQESDRHPNVLRYFCTERGPQFHYIALELCRASLQEYVENPDLDRGGLEPEVVLQQLMSGLAHLHSLHIVHRDLKPGNILITGPDSQGLGRVVLSDFGLCKKLPAGRCSFSLHSGIPGTEGWMAPELLQLLPPDSPTSAVDIFSAGCVFYYVLSGGSHPFGDSLYRQANILTGAPCLAHLEEEVHDKVVARDLVGAMLSPLPQPRPSAPQVLAHPFFWSRAKQLQFFQDVSDWLEKESEQEPLVRALEAGGCAVVRDNWHEHISMPLQTDLRKFRSYKGTSVRDLLRAVRNKKHHYRELPVEVRQALGQVPDGFVQYFTNRFPRLLLHTHRAMRSCASESLFLPYYPPDSEARRPCPGATGR

SEQ ID NO: 15 - protein, 641 aa, Homo sapiens
  misc_feature: Incyte ID No 7163416CD1
MFRKKKKKRPEISAPQNFQHRVHTSFDPKEGKFVGLPPQWQNILDTLRRPKPVVDPSRITRVQLQPMKTVVRGSAMPVDGYISGLLNDIQKLSVISSNTLRGRSPTSRRRAQSLGLLGDEHWATDPDMYLQSPQSERTDPHGLYLSCNGGTPAGHKQMPWPEPQSPRVLPNGLAAKAQSLGPAEFQGASQRCLQLGACLQSSPPGASPPTGTNRHGMKAAKHGSEEARPQSCLVGSATGRPGGEGSPSPKTRESSLKRRLFRSMFLSTAATAPPSSSKPGPPPQSKPNSSFRPPQKDNPPSLVAKAQSLPSDQPVGTFSPLTTSDTSSPQKSLRTAPATGQLPGRSSPAGSPRTWHAQISTSNLYLPQDPTVAKGALAGEDTGVVTHEQFKAALRMVVDQGDPRLLLDSYVKIGEGSTGIVCLAREKHSGRQVAVKMMDLRKQQRRELLFNEVVIMRDYQHFNVVEMYKSYLVGEELWVLMEFLQGGALTDIVSQVRLNEEQIATVCEAVLQALAYLHAQGVIHRDIKSDSILLTLDGRVKLSDFGFCAQISKDVPKRKSLVGTPYWMAPEVISRSLYATEVDIWSLGIMVIEMVDGEPPYFSDSPVQAMKRLRDSPPPKLKNSHKVSWHTRVRPRRPHSS

SEQ ID NO: 16 - protein, 576 aa, Homo sapiens
  misc_feature: Incyte ID No 7472822CD1
MPALSTGSGSDTGLYELLAALPAQLQPHVDSQEDLTFLWDMFGEKSLHSLVKIHEKLHYYEKQSPVPILHGAAALADDLAEELQNKPLNSEIRELLKLLSKPNVKALLSVHDTVAQKNYDPVLPPMPEDIDDEEDSVKIIRLVKNREPLGATIKKDEQTGAIIVARIMRGGAADRSGLIHVGDELREVNGIPVEDKRPEEIIQILAQSQGAITFKIIPGSKEETPSKEGKMFIKALFDYNPNEDKAIPCKEAGLSFKKGDILQIMSQDDATWWQAKHEADANPRAGLIPSKHFQERRLALRRPEILVQPLKVSNRKSSGFRKSFRLSRKDKKTNKSMYECKKSDQYDTADVPTYEEVTPYRRQTNEKYRLVVLVGPVGVGLNELKRKLLISDTQHYGVTVPHTTRARRSQESDGVEYIFISKHLFETDVQNNKFIEYGEYKNNYYGTSIDSVRSVLAKNKVCLLDVQPHTVKHLRTLEFKPYVIFIKPPSIERLRETRKNAKIISSRDDQGAAKPFTEEDFQEMIKSAQIMESQYGHLFDKIIINDDLTVAFNELKTTFDKLETETHWVPVSWLHS

SEQ ID NO: 17 - protein, 794 aa, Homo sapiens
  misc_feature: Incyte ID No 7477486CD1
MVAGLTLGKGPESPDGDVSVPERKDEVAGGGGEEEEAEERGRHAQYVGPYRLEKTLGKGQTGLVKLGVHCITGQKVAIKIVNREKLSESVLMKVEREIAILKLIEHPHVLKLHDVYENKKYLYLVLEHVSGGELFDYLVKKGRLTPKEARKFFRQIVSALDFCHSYSICHRDLKPENLLLDEKNNIRIADFGMASLQVGDSLLETSCGSPHYACPEVIKGEKYDGRRADMWSCGVILFALLVGALPFDDDNLRQLLEKVKRGVFHMPHFIPPDCQSLLRGMIEVEPEKRLSLEQIQKHPWYLGGKHEPDPCLEPAPGRRVAMRSLPSNGELDPDVLESMASLGCFRDRERLHRELRSEEENQEKMIYYLLLDRKERYPSCEDQDLPPRNDVDPPRKRVDSPMLSRHGKRRPERKSMEVLSITDAGGGGSPVPTRRALEMAQHSQRSRSVSGASTGLSSSPLSSPRSPVFSFSPEPGAGDEARGGGSPTSKTQTLPSRGPRGGGAGEQPPPPSARSTPLPGPPGSPRSSGGTPLHSPLHTPRASPTGTPGTTPPPSPGGGVGGAAWRSRLNSIRNSFLGSPRFHRRKMQVPTAEEMSSLTPESSPELAKRSWFGNFISLDKEEQIFLVLKDKPLSSIKADIVHAFLSIPSLSHSVLSQTSFRAEYKASGGPSVFQKPVRFQVDISSSEGPEPSPRRDGSGGGGIYSVTFTLISGPSRRFKRVVETIQAQLLSTHDQPSVQALADEKNGAQTRPAGAPPRSLQPPPGRPDPELSSSPRRGPPKDKKLLATNGTPLP

SEQ ID NO: 18 - protein, 504 aa, Homo sapiens
  misc_feature: Incyte ID No 3773709CD1
MSGLLTDPEQRAQEPRYPGFVLGLDVGSSVIRCHVYDRAARVCGSSVQKVENLYPQIGWVEIDPDVLWIQFVAVIKEAVKAAGIQMNQIVGLGISTQRATFITWNKKTGNHFHNFISWQDLRAVELVKSWNNSLLMKIFHSSCRVLHFFTRSKRLFTASLFTFTTQQTSLRLVWILQNLTEVQKAVEEENCCFGTIDTWWLYKLTKGSVYATDFSNASTTGLFDPYSHNFGSVDEEIFGVPIPIVALVADQQSAMFGECCFQTGDVKLTMGTGTFLDINTGNSLQQTTGGFYPLIGWKIGQEVVCLAESNAGDTGTAIKWAQQLDLFTDAAETEKMAKSLEDSEGVCFVPSFSGLQAPLNDPWACASFMGLKPSTSKYHLVRAILESIAFRNKQLYEMMKKEIHIPVRKIRADGGVCKNGFVMQMTSDLINENIDRPADIDMSCLGAASLAGLAVGFWTDKEELKKLRQSEVVFKPQKKCQEYEMSLENWAKAVKRSMNWYNKT

SEQ ID NO: 19 - protein, 553 aa, Homo sapiens
  misc_feature: Incyte ID No 7477204CD1
MVDMGALDNLIANTAYLQARKPSDCDSKELQRRRRSLALPGLQGCAELRQKLSLNFHSLCEQQPIGRRLFRDFLATVPTFRKAATFLEDVQNWELAEEGPTKDSALQGLVATCASAPAPGNPQPFLSQAVATKCQAATTEEERVAAVTLAKAEAMAFLQEQPFKDFVTSAFYDKFLQWKLFEMQPVSDKYFTEFRVLGKGGFGEVCAVQVKNTGKMYACKKLDKKRLKKKGGEKMALLEKEILEKVSSPFIVSLAYAFESKTHLCLVMSLMNGGDLKFHIYNVGTRGLDMSRVIFYSAQIACGMLHLHELGIVYRDMKPENVLLDDLGNCRLSDLGLAVEMKGGKPITQRAGTNGYMAPEILMEKVSYSYPVDWFAMGCSIYEMVAGRTPFKDYKEKVSKEDLKQRTLQDEVKFQHDNFTEEAKDICRLFLAKKPEQRLGSREKSDDPRKHHFFKTINFPRLEAGLIEPPFVPDPSVVYAKDIAEIDDFSEVRGVEFDDKDKQFFKNFATGAVPIAWQEEIIETGLFEELNDPNRPTGCEEGNSSKSGVCLLL

SEQ ID NO: 20 - protein, 871 aa, Homo sapiens
  misc_feature: Incyte ID No 3016969CD1
MGPGDISLPGRPKPGPCSSPGSASQASSSQVSSLRVGSSQVGTEPGPSLDAEGWTQEAEDLSDSTPTLQRPQEQVTMRKFSLGGRGGYAGVAGYGTFAFGGDAGGMLGQGPMWARIAWAVSQSEEEEQEEARAESQSEEQQEARAESPLPQVSARPVPEVGRAPTRSSPEPTPWEDIGQVSLVQIRDLSGDAEAADTISLDISEVDPAYLNLSDLYDIKYLPFEFMIFRKVPKSAQPEPPSPMAEEELAEFPEPTWPWPGELGPHAGLEITEESEDVDALLAEAAVGRKRKWSSPSRSLFHFPGRHLPLDEPAELGLRERVKASVEHISRILKGRPEGLEKEGPPRKKPGLASFRLSGLKSWDRAPTFLRELSDETVVLGQSVTLACQVSAQPAAQATWSKDGAPLESSSRVLISATLKNFQLLTILVVVAEDLGVYTCSVSNALGTVTTTGVLRKAERPSSSPCPDIGEVYADGVLLVWKPVESYGPVTYIVQCSLEGGSWTTLASDIFDCCYLTSKLSRGGTYTFRTACVSKAGMGPYSSPSEQVLLGGPSHLASEEESQGRSAQPLPSTKTFAFQTQIQRGRFSVVRQCWEKASGRALAAKIIPYHPKDKTAVLREYEALKGLRHPHLAQLHAAYLSPRHLVLILELCSGPELLPCLAERASYSESEVKDYLWQMLSATQYLHNQHILHLDLRSENMIITEYNLLKVVDLGNAQSLSQEKVLPSDKFKDYLETMAPELLEGQGAVPQTDIWAIGVTAFIMLSAEYPVSSEGARDLQRGLRKGLVRLSRCYAGLSGGAVAFLRSTLCAQPWGRPCASSCLQCPWLTEEGPACSRPAPVTFPTARLRVFVRNREKRRALLYKRHNLAQVR

SEQ ID NO: 21 - protein, 765 aa, Homo sapiens
  misc_feature: Incyte ID No 063497CD1
MAGFKRGYDGKIAGLYDLDKTLGRGHFAVVKLARHVFTGEKVAVKVIDKTKLDTLATGHLFQEVRCMKLVQHPNIVRLYEVIDTQTKLYLILELGDGGDMFDYIMKHEEGLNEDLAKKYFAQIVHAISYCHKLHVVHRDLKPENVVFFEKQGLVKLTDFGFSNKFQPGKKLTTSCGSLAYSAPEILLGDEYDAPAVDIWSLGVILFMLVCGQPPFQEANDSETLTMIMDCKYTVPSHVSKECKDLITRMLQRDPKRRASLEEIENHPWLQGVDPSPATKYNIPLVSYKNLSEEEHNSIIQRMVLGDIADRDAIVEALETNRYNHITATYFLLAERILREKQEKEIQTRSASPSNIKAQFRQSWPTKIDVPQDLEDDLTATPLSHATVPQSPARAADSVLNGHRSKGLCDSAKKDDLPELAGPALSTVPPASLKPTASGRKCLFRVEEDEEEDEEDKKPMSLSTQVVLRRKPSVTNRLTSRKSAPVLNQIFEEGESDDEFDMDENLPPKLSRLKMNIASPGTVHKRYHRRKSQGRGSSCSSSETSDDDSESRRRLDKDSGFTYSWHRRDSSEGPPGSEGDGGGQSKPSNASGGVDKASPSENNAGGGSPSSGSGGNPTNTSGTTRRCAGPSNSMQLASRSAGELVESLKLMSLCLGSQLHGSTKYIIDPQNGLSFSSVKVQEKSTWKMCISSTGNAGQVPAVGGIKFFSDHMADTTTELERIKSKNLKNNVLQLPLCEKTISVNIQRNPKEGLLCASSPASCCHVI

SEQ ID NO: 22 - protein, 588 aa, Homo sapiens
  misc_feature: Incyte ID No 1625436CD1
MATTATCTRFTDDYQLFEELGKGAFSVVRRCVKKTSTQEYAAKIINTKKLSARDHQKLEREARICRLLKHPNIVRLHDSISEEGFHYLVFDLVTGGELFEDIVAREYYSEADASHCIHQILESVNHIHQHDIVHRDLKPENLLLASKCKGAAVKLADFGLAIEVQGEQQAWFGFAGTPGYLSPEVLRKDPYGKPVDIWACGVILYILLVGYPPFWDEDQHKLYQQIKAGAYDFPSPEWDTVTPEAKNLINQMLTINPAKRITADQALKYPWVCQRSTVASMMHRQETVECLRKFNARRKLKGAILTTMLVSRNFSVGRQSSAPASPAASAAGLAGQAAKSLLNKKSDGGVKKRKSSSSVHLMPQSNNKNSLVSPAQEPAPLQTAMEPQTTVVHNATDGIKGSTESCNTTTEDEDLKAAPLRTGNGSSVPEGRSSRDRTAPSAGMQPQPSLCSSAMRKQEIIKITEQLIEAINNGDFEAYTKICDPGLTSFEPEALGNLVEGMDFHKFYFENLLSKNSKPIHTTILNPHVHVIGEDAACIAYIRLTQYIDGQGRPRTSQSEETRVWHRRDGKWLNVHYHCSGAPAAPLQ

SEQ ID NO: 23 - protein, 1798 aa, Homo sapiens
  misc_feature: Incyte ID No 3330646CD1
MKRSRCRDRPQPPPPDRREDGVQRAAELSQSLPPRRRAPPGRQRLEERTGPAGPEGKEQDVATGVSPLLFRKLSNPDIFSSTGKVKLQRQLSQDDCKLWRGNLASSLSGKQLLPLSSSVHSSVGQVTWQSSGEASNLVRMRNQSLGQSAPSLTAGLKELSLPRRGSFCRTSNRKSLIVTSSTSPTLPRPHSPLHGHTGNSPLDSPRNFSPNAPAHFSFVPARRTDGRRWSLASLPSSGYGTNTPSSTVSSSCSSQEKLHQLPFQPTADELHFLTKHFSTESVPDEEGRQSPAMRPRSRSLSPGRSPVSFDSEIIMMNHVYKERFPKATAQMEERLAEFISSNTPDSVLPLADGALSFIHHQVIEMARDCLDKSRSGLITSQYFYELQENLEKLLQDAHERSESSEVAFVMQLVKKLMIIIARPARLLECLEFDPEEFYHLLEAAEGHAKEGQGIKCDIPRYIVSQLGLTRDPLEEMAQLSSCDSPDTPETDDSIEGHGASLPSKKTPSEEDFETIKLISNGAYGAVFLVRHKSTRQRFAMKKINKQNLILRNQIQQAFVERDILTFAENPFVVSMFCSFDTKRHLCMVMEYVEGGDCATLLKNIGALPVDMVRLYFAETVLALEYLHNYGIVHRDLKPDNLLITSMGHIKLTDFGLSKMGLMSLTTNLYEGHIEKDAREFLDKQVCGTPEYIAPEVILRQGYGKPVDWWAMGIILYEFLVGCVPFFGDTPEELFGQVISDEIVWPEGDEALPPDAQDLTSKLLHQNPLERLGTGSAYEVKQHPFFTGLDWTGLLRQKAEFIPQLESEDDTSYFDTRSERYHHMDSEDEEEVSEDGCLEIRQFSSCSPRFNKVYSSMERLSLLEERRTPPPTKRSLSEEKEDHSDGLAGLKGRDRSWVIGSPEILRKRLSVSESSHTESDSSPPMTVRRRCSGLLDAPRFPEGPEEASSTLRRQPQEGIWVLTPPSGEGVSGPVTEHSGEQRPKLDEEAVGRSSGSSPAMETRGRGTSQLAEGATAKAISDLAVRRARHRLLSGDSTEKRTARPVNKVIKSASATALSLLIPSEHHTCSPLASPMSPHSQSSNPSSRDSSPSRDFLPALGSMRPPIIIHRAGKKYGFTLRAIRVYMGDSDVYTVHHMVWHVEDGGPASEAGLRQGDLITHVNGEPVHGLVHTEVVELILKSGNKVAISTTPLENTSIKVGPARKGSYKAKMARRSKRSRGKDGQESRKRSSLFRKITKQASLLHTSRSLSSLNRSLSSGESGPGSPTHSHSLSPRSPTQGYRVTPDAVHSVGGNSSQSSSPSSSVPSSPAGSGHTRPSSLHGLAPKLQRQYRSPRRKSAGSIPLSPLAHTPSPPPPTASPQRSPSPLSGHVAQAFPTKLHLSPPLGRQLSRPKSAEPPRSPLLKRVQSAEKLAAALAASEKKLATSRKHSLDLPHSELKKELPPREVSPLEVVGARSVLSGKGALPGKGVLQPAPSRALGTLRQDRAERRESLQKQEAIREVDSSEDDTEEGPENSQGAQELSLAPHPEVSQSVAPKGAGESGEEDPFPSRDPRSLGPMVPSLLTGITLGPPRMESPSGPHRRLGSPQAIEEAASSSSAGPNLGQSGATDPIPPEGCWKAQHLHTQALTALSPSTSGLTPTSSCSPPSSTSGKLSMWSWKSLIEGPDRASPSRKATMAGGLANLQDLENTTPAQPKNLSPREQGKTQPPSAPRLAHPSYEDPSQGWLWESECAQAVKEDPALSITQVPDASGDRRQDVPCRGCPLTQKSEPSLRRGQEPGGHQKHRDLALVPDELLKQT

SEQ ID NO: 24 - protein, 362 aa, Homo sapiens [sequence omitted]

SEQ ID NO: 25 - protein, 275 aa, Homo sapiens
  misc_feature: Incyte ID No 621293CD1
MVPEDISELETAQKLLEYHRNIVRVIPSYPKILKVISADQPCVDVFYQALTYVQSNHRTNAPFTPRVLLLGPVGSGKSLQAALLAQKYRLVNVCCGQLLKEAVADRTTFGELIQPFFEKEMAVPDSLLMKVLSQRLDQQDCIQKGWVLHGVPRDLDQAHLLNRLGYNPNRVFFLNVPFDSIMERLTLRRIDPVTGERYHLMYKPPPTMEIQARLLQNPKDAEEQVKLKMDLFYRNSADLEQLYGSAITLNGDQDPYTVFEYIESGIINPLPKKIP

SEQ ID NO: 26 - protein, 660 aa, Homo sapiens
  misc_feature: Incyte ID No 7480774CD1
MRLEAPRGGRRRQPGQQRPGPGAGAPAGRPEGGGPWARTEESSLHSEPERAGLGPAPGTESPQAEFWTDGQTEPAAAGLGVETERPKQKTEPDRSSLRTHLEWSWSELETTCLWTETGTDGLWTDPHRSDLQFQPEEASPWTQPGVHGPWTELETHGSQTQPERVKSWADNLWTHQNSSSLQTHPEGACPSKEPSADGSWKELYTDGSRTQQDIEGPWTEPYTDGSQKKQDTEAARKQPGTGGFQIQQDTDGSWTQPSTDGSQTAPGTDCLLGEPEDGPLEEPEPGELLTHLYSHLKCSPLCPVPRLIITPETPEPEAQPVGPPSRVEGGSGGFSSASSFDESEDDVVAGGGGASDPEDRSGSKPWKKLKTVLKYSPFVVSFRKHYPWVQLSGHAGNFQAGEDGRILKRFCQCEQRSLEQLMKDPLRPFVPAYYGMVLQDGQTFNQMEDLLADFEGPSIMDCKMGSRTYLEEELVKARERPRPRKDMYEKMVAVDPGAPTPEEHAQGAVTKPRYMQWRETMSSTSTLGFRIEGIKKADGTCNTNFKKTQALEQVTKVLEDFVDGDHVILQKYVACLEELREALEISPFFKTHEVVGSSLLFVHDHTGLAKVWMIDFGKTVALPDHQTLSHRLPWAEGNREDGYLWGLDNMICLLQGLAQS

SEQ ID NO: 27 - DNA, 822 bp, Homo sapiens
  misc_feature: Incyte ID No 2011384CB1
atgtcgggagacaaacttctgagcgaactcggttataagctgggccgcacaattggagagggcagctactccaaggtgaaggtggccacatccaagaagtacaagggtaccgtggccatcaaggtggtggaccggcggcgagcgcccccggacttcgtcaacaagttcctgccgcgagagctgtccatcctgcggggcgtgcgacacccgcacatcgtgcacgtcttcgagttcatcgaggtgtgcaacgggaaactgtacatcgtgatggaagcggccgccaccgacctgctgcaagccgtgcagcgcaacgggcgcatccccggagttcaggcgcgcgacctctttgcgcagatcgccggcgccgtgcgctacctgcacgatcatcacctggtgcaccgcgacctcaagtgcgaaaacgtgctgctgagcccggacgagcgccgcgtcaagctcaccgacttcggcttcggccgccaggcccatggctacccagacctgagcaccacctactgcggctcagccgcctacgcgtcacccgaggtgctcctgggcatcccctacgaccccaagaagtacgatgtgtggagcatgggcgtcgtgctctacgtcatggtcaccgggtgcatgcccttcgacgactcggacatcgccggcctgccccggcgccagaaacgcggcgtgctctatcccgaaggcctcgagctgtccgagcgctgcaaggccctgatcgccgagctgctgcagttcagcccgtccgccaggccctccgcgggccaggtagcgcgcaactgctggctgcgcgccggggactccggctag

SEQ ID NO: 28 - DNA, 1376 bp, Homo sapiens
  misc_feature: Incyte ID No 2004888CB1
gcttattgaatatttaaataagagtcccagtgtggatcacttgctatccattaagaagacattgaaaagcttaaaagctctactcagatggaaattggttgaaaagagtaatttggaagagtcagatgatcctgatggctctcaaattgagaaaataaaagaagaaataactcagctgcgcaataatgtctttcaggaaatttatcatgagagagaggaatatgagatgctaactagtttggcacagaaatggttccctgagctgcctctgcttcatcctgaaataggattactcaaatacatgaactctggtggtctccttacaatgagcttggaacgagatcttcttgatgctgagcccatgaaggaacttagcagcaagcgtcctttggtacgttctgaggttaatgggcagataattctgttaaagggctattctgtggatgttgacacagaagccaaggtgattgagagagcagccacctaccatagagcttggagagaagctgaaggagactcagggttactgccattgatattcctgtttttatgtaagtctgatcctatggcttatctgatggtcccatactaccctagggcaaacctgaatgctgttcaagccaacatgcctttaaattcagaagaaactttaaaggtcatgaaaggtgttgcccagggtctgcatacattgcataaggctgacataattcatggatcacttcatcagaacaatgtatttgctttaaaccgtgaacaaggaattgttggagattttgacttcaccaaatctgtgagtcagcgagcctcggtgaacatgatggttggtgacttgagtttgatgtcacctgagttgaaaatgggaaaacctgcttctccaggttcagacttatatgcttatggctgcctcttattatggctttctgttcaaaatcaggagtttgagataaataaagatggaatccccaaagtggatcagtttcatctggatgataaagtcaaatccctcctctgtagcttgatatgttatagaagttcaatgactgctgaacaagttttaaatgctgaatgtttcttgatgccaaaggagcaatcagttccaaacccagaaaaagatactgaatacaccctatataaaaaggaagaagaaataaagacggagaacttggataaatgtatggagaagacaagaaatggtgaagccaactttgattgttaaattattattgttgttgttgcagaggttctttttaaaaactttggtttggttaatacacagaaatatctagaaatgttctgggactagttgagttgtatctttagtattcaggttgaagaaaaataaagatgtgtggtatactagttctgatgngctgtgc

SEQ ID NO: 29 - DNA, 3468 bp, Homo sapiens
  misc_feature: Incyte ID No 2258952CB1
ttccactataacctttctctagggtcaaagagatgatgagtgacaccagcacgttccccaatcacccttcctcccctgctgcatccccatctgggggaaggggagtcatggccagccctgcttgggacaggagcaaagggtggtcccagacccctcagagagctgactttgtctctacccccttgcaggttcatactctcaggccagagaacctcctgctggtgtccaccttggatggaagtctccacgcactaagcaagcagacaggggacctgaagtggactctgagggatgatcccgtcatcgaaggaccaatgtacgtcacagaaatggcctttctctctgacccagcagatggcagcctgtacatcttggggacccaaaaacaacagggattaatgaaactgccattcaccatccctgagctggttcatgcctctccctgccgcagctctgatggggtcttctacacaggccggaagcaggatgcctggtttgtggtggaccctgagtcaggggagacccagatgacactgaccacagagggtccctccaccccccgcctctacattggccgaacacagtatacggtcaccatgcatgacccaagagccccagccctgcgctggaacaccacctaccgccgctactcagcgccccccatggatggctcacctgggaaatacatgagccacctggcgtcctgcgggatgggcctgctgctcactgtggacccaggaagcgggacggtgctgtggacacaggacctgggcgtgcctgtgatgggcgtctacacctggcaccaggacggcctgcgccagctgccgcatctcacgctggctcgagacactctgcatttcctcgccctccgctggggccacatccgactgcctgcctcaggcccccgggacacagccaccctcttctctaccttggacacccagctgctaatgacgctgtatgtggggaaggatgaaactggcttctatgtctctaaagcactggtccacacaggagtggccctggtgcctcgtggactgaccctggcccccgcagatggccccaccacagatgaggtgacactccaagtctcaggagagcgagagggctcacccagcactgctgttagatacccctcaggcagtgtggccctcccaagccagtggctgctcattggacaccacgagctacccccagtcctgcacaccaccatgctgagggtccatcccaccctggggagtggaactgcagagacaagacctccagagaatacccaggccccagccttcttcttggagctattgagcctgagccgagagaaactttgggactccgagctgcatccagaagaaaaaactccagactcttacttggggctgggaccccaagacctgctggcagctagcctcactgctgtcctcctgggagggtggattctctttgtgatgaggcagcaacaggagacccccctggcacctgcagactttgctcacatctcccaggatgcccagtccctgcactcgggggccagccggaggagccagaagaggcttcagagtccctcacctgagtcaccaccctcctctcccccagctgagcaactcaccgtagtggggaagatttccttcaatcccaaggacgtgctgggccgcggggcaggcgggactttcgttttcaggggacagtttgagggacgggcagtggctgtcaagcggctcctccgcgagtgctttggcctggttcggcgggaagttcaactgctgcaggagtctgacaggcaccccaacgtgctccgctacttctgcaccgagcggggaccccagttccactacattgccctggagctctgccgggcctccttgcaggagtacgtagaaaacccggacctggatcgcgggggtctggagcccgaggtcgtgctgcagcagctgatgtctggcctggcccacctgcactctttacacatagtgcaccgggacctgaagccaggaaatattctcatcaccgggcctgacagccagggcctgggcagagtggtgctctcagacttcggcctctgcaagaagctgcctgctggccgctgtagcttcagcctccactccggcatccccggcacggaaggctggatggcgcccgagcttctgcagctcctgccaccagacagtcctaccagcgctgtggacatcttctctgcaggctgcgtgttctactacgtgctttctggtggcagccacccctttggagacagtctttatcgccaggcaaacatcctcacaggggctccctgtctggctcacctggaggaagaggtccacgacaaggtggttgcccgggacctggttggagccatgttgagcccactgccgcagccacgcccctctgccccccaggtgctggcccaccccttcttttggagcagagccaagcaactccagttcttccaggacgtcagtgactggctggagaaggagtccgagcaggagcccctggtgagggcactggaggcgggaggctgcgcagtggtccgggacaactggcacgagcacatctccatgccgctgcagacagatctgagaaagttccggtcctataaggggacatcagtgcgagacctgctccgtgctgtgaggaacaagaagcaccactacagggagctcccagttgaggtgcgacaggcactcggccaagtccctgatggcttcgtccagtacttcacaaaccgcttcccacggctgctcctccacacgcaccgagccatgaggagctgcgcctctgagagcctcttcctgccctactacccgccagactcagaggccaggaggccatgccctggggccacagggaggtgaggtgggctggatgccacacagatggtctccgtgctggctcactgaagagctgagcctgtggctggcctcagaatcaggctgggtgcagtggctcacacctgtaatcccagcattttgggaggctgagtgagaggatcacttgagctcaggagttcgagaccagcctggccaacatggcaacaccccatttctacaaaaaatttgtaaaattagccaggcatggtggcgcacgcctgtagtcccagctgcttgggaggctgaggtgggagaatcacttgagcccaggagttcgaggctgcagtgagccaggatcatgccactgcactccagcctggtccacagagagacactgtcaccccctttcccccacaagactggcagaggctgggcagcctggggctgatgaagcagagatgttcgctggatcccaggccctggcacccctcaggaaatacaagaaaaagaatattcacatctgtttaatgtgcataaagccaaggaaaggacagttccgaattcaaaaaaaaaaaaaaaaaaaaaaaaaaaaaaaaaaaaaaaaaaaaaaaaaaaaaaaaaaaaaaaaaaagaagaaaaaaaaaaaaaagaagaagacccagac

SEQ ID NO: 30 - DNA, 2831 bp, Homo sapiens
  misc_feature: Incyte ID No 7473244CB1
cttctcccgccggggccgcttgttgcaccgccccgcggcctgcgggagccgctcgccccggccttgtgctcgcgtccgcacccctttcctgtcgccccccggggcccgcaccacagcccggccggcgagaccccggccagaccccgctgcccgcacaaaatgtcggcccggacgccattgccgacggtgaacgagcgggacacggaaaatcatacatctgtggatggatatactgaaccacacatccagcctaccaagtcgagtagcagacagaacatcccccggtgtagaaactccattacgtcagcaacagatgaacagcctcacattggaaattaccgtttacaaaaaacaatagggaagggaaattttgccaaagtcaaattggcaagacacgttctaactggtagagaggttgctgtgaaaataatagacaaaactcagctaaatcctaccagtctacaaaagttatttcgagaagtacgaataatgaagatactgaatcatcctaatatagtaaaattgtttgaagttattgaaacagagaagactctctatttagtcatggaatacgcgagtgggggtgaagtatttgattacttagttgcccatggaagaatgaaagagaaagaggcccgtgcaaaatttaggcagattgtatctgctgtacagtattgtcatcaaaagtacattgttcaccgtgatcttaaggctgaaaaccttctccttgatggtgatatgaatattaaaattgctgactttggttttagtaatgaatttacagttgggaacaaattggacacattttgtggaagcccaccctatgctgctcccgagcttttccaaggaaagaagtatgatgggcctgaagtggatgtgtggagtctgggcgtcattctctatacattagtcagtggctccttgcctttcgatggccagaatttaaaggaactgcgagagcgagttttacgagggaagtaccgtattcccttctatatgtccacagactgtgaaaatcttctgaagaaattattagtcctgaatccaataaagagaggcagcttggaacaaataatgaaagatcgatggatgaatgttggtcatgaagaggaagaactaaagccatatactgagcctgatccggatttcaatgacacaaaaagaatagacattatggtcaccatgggctttgcacgagatgaaataaatgatgccttaataaatcagaagtatgatgaagttatggctacttatattcttctaggtagaaaaccacctgaatttgaaggtggtgaatcgttatccagtggaaacttgtgtcagaggtcccggcccagtagtgacttaaacaacagcactcttcagtcccctgctcacctgaaggtccagagaagtatctcagcaaatcagaagcagcggcgtttcagtgatcatgctggtccatccattcctcctgctgtatcatataccaaaagacctcaggctaacagtgtggaaagtgaacagaaagaggagtgggacaaagatgtggctcgaaaacttggcagcacaacagttggatcaaaaagcgagatgactgcaagccctcttgtagggccagagaggaaaaaatcttcaactattccaagtaacaatgtgtattctggaggtagcatggcaagaaggaatacatatgtctgtgaaaggaccacagatcgatacgtagcattgcagaatggaaaagacagcagccttacggagatgtctgtgagtagcatatcttctgcaggctcttctgtggcctctgctgtcccctcagcacgaccccgccaccagaagtccatgtccacttctggtcatcctattaaagtcacactgccaaccattaaagacggctctgaagcttaccggcctggtacaacccagagagtgcctgctgcttccccatctgctcacagtattagtactgcgactccagaccggacccgttttccccgagggagctcaagccgaagcactttccatggtgaacagctccgggagcgacgcagcgttgcttataatgggccacctgcttcaccatcccatgaaacgggtgcatttgcacatgccagaaggggaacgtcaactggtataataagcaaaatcacatccaaatttgttcgcagggatccaagtgaaggcgaagccagtggcagaaccgacacctcaagaagtacatcaggggaaccaaaagaaagagacaaggaagagggtaaagattctaagccgcgttctttgcggttcacatggagtatgaagaccactagttcaatggaccctaatgacatgatgagagaaatccgaaaagtgttagatgcaaataactgtgattatgagcaaaaagagagatttttgcttttctgtgtccatggagacgctagacaggatagcctcgtgcagtgggagatggaagtctgcaagttgccacgactgtcacttaatggggttcgcttcaagcgaatatctgggacatctattgcctttaagaacattgcatcaaaaatagcaaatgagcttaagctgtaaagaagtccaaatttacaggttcagggaagatacatacatatatgaggtacagtttttgaatgtactggtaatgcctaatgtggtctgcctgtgaatctccccatgtagaatttgcccttaatgcaataaggttatacatagttatgaactgtaaaattaaagtcagtatgaactataataaatatctgtagcttaaaaagtaggttcacatgtacaggtaagtatattgtgtatttctgttcattttctgttcatagagttgtataataaaacatgattgcttt

SEQ ID NO: 31 - DNA, 2693 bp, Homo sapiens
  misc_feature: Incyte ID No 1242491CB1
agtgtgctggaaagattgcccctgacttgatttggctgacctgcctagaaatattatgttgaataatgatgagttggaatttgaacaagctccagagttttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaacggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctagg

SEQ ID NO: 32 - DNA, 2973 bp, Homo sapiens
  misc_feature: Incyte ID No 2634875CB1
agtgtgctggaaagactgcccacacccctgcctccgcctctgcccacccggcccaatcccttacaactgcccaggactgctcctgagcagccgctgggagacagacggcaaccaggttgcccctctttgctccaggtacctctctcccctcagttagcaggcctcggcttcctgtctcactgcagccagacgagaggggaaattggacagcctgacacactccactcttgtttctgcagctagaaagacttgagttagacaagcagcagcacacgcctccctacctcatggcgacagaaaatggagcagttgagctgggaattcagaacccatcaacagacaaggcacctaaaggtcccacaggtgaaagacccctggctgcagggaaagaccctggccccccagacccaaagaaagctccggatccacccaccctgaagaaagatgccaaagcccctgcctcagagaaaggggatggtaccctggcccaaccctcaactagcagccaaggccccaaaggagagggtgacaggggcggggggcccgcggagggcagtgctgggcccccggcagccctgccccagcagactgcgacacctgagaccagcgtcaagaagcccaaggctgagcagggagcctcaggcagccaggatcctggaaagcccagggtgggcaagaaggcagcagagggccaagcagcagccaggaggggctcacctgcctttctgcatagccccagctgtcctgccatcatctccagttctgagaagctgctggccaagaagcccccaagcgaggcatcagagctcacctttgaaggggtgcccatgacccacagccccacggatcccaggccagccaaggcagaagaaggaaagaacatcctggcagagagccagaaggaagtgggagagaaaaccccaggccaggctggccaggctaagatgcaaggggacacctcgagggggattgagttccaggctgttccctcagagaaatccgaggtggggcaggccctctgtctcacagccagggaggaggactgcttccagattttggatgattgcccgccacctccggcccccttccctcaccgcatggtggagctgaggaccgggaatgtcagcagtgaattcagtatgaactccaaggaggcgctcggaggtggcaagtttggggcagtctgtacctgcatggagaaagccacaggcctcaagctggcagccaaggtcatcaagaaacagactcccaaagacaaggaaatggtgttgctggagattgaggtcatgaaccagctgaaccaccgcaatctgatccagctgtatgcagccatcgagactccgcatgagatcgtcctgttcatggagtacatcgagggcggagagctcttcgagaggattgtggatgaggactaccatctgaccgaggtggacaccatggtgtttgtcaggcagatctgtgacgggatcctcttcagtgtgctggaaagggttttgcacctggacctcaagccagagaacatcctgtgtgtcaacaccaccgggcatttggtgaagatcattgactttggcctggcacggaggtataaccccaacgagaagctgaaggtgaactttgggaccccagagttcctgtcacctgaggtggtgaagggtgaccaaatctccgataagacagacatgtggagtatgggggtgatcacctacatgctgctgagcggcctctcccccttcctgggagatgatgacacagagaccctaaacaacgttctatctggcaactggtactttgatgaagagacctttgaggccgtatcagacgaggccaaagactttgtctccaacctcatcgtcaaggaccagagggcccggatgaacgctgcccagtgtctcgcccatccctggctcaacaacctggcggagaaagccaaacgctgtaaccgacgccttaagtcccagatcttgcttaagaaatacctcatgaagaggcgctggaagaaaaacttcattgctgtcagcgctgccaaccgcttcaagaagatcagcagctcgggggcactgatggctctgggggtctgagccctgggcgcagctgaagcctggacgcagccacacagtggccggggctgaagccacacagcccagaaggccagaaaaggcagccagatccccagggcagcctcgttaggacaaggctgtgccaggctgggaggctcggggctccccacgcccccatgcagtgaccgcttccccgatgtgagccgcctcggagtgtggcctggatccatcctgctagcacctccccagacagggctccagcctgtcggccacaccccagactccaggcccccgttgaagccgctcccggttccctccccagctcctcgtctttgaactgccgccgccgtggtgacccctgctttgccccactgggagagtccttagcctgggcctcctcctagctggagtgccatggctggggggtctcagcatgtagggcttctgtggttgtggatgggaggctcctggtggggcagaaaggctgcaacgctgattcctaaggcccagctgccagggaagacagagcaggctttgtgagagaggacctccatgcccccgccacctccccactccagcagataaggccgagcccacaccatctggcccaggctggcccccacccaccttccttgcgaccaccaacacacaggaactctgtgtgagagagagggcgcccagcccaggcctggtggagggggaggggagaagccaagggacacaggagaccacccccgagcttgcctcagggccaagccggcccaacccaaccactcggggcccccatcttgggggtcacccatggcctcagatgatggggtcagcaggcccaggagaattaggaaggccatggggcagcctccagtctgctctcagcttgtgccttgtaaataaatgtacaggttggaaaaaaaaa

SEQ ID NO: 33 - DNA, 2066 bp, Homo sapiens
  misc_feature: Incyte ID No 3951059CB1
cgccagtggggagatgttgaagttcaaatatggagcgcggaatcctttggatgctggtgctgctgaacccattgccagccgggcctccaggctgaatctgttcttccaggggaaaccaccctttatgactcaacagcagatgtctcctctttcccgagaagggatattagatgccctctttgttctctttgaagaatgcagtcagcctgctctgatgaagattaagcacgtgagcaactttgtccggaagtattccgacaccatagctgagttacaggagctccagccttcggcaaaggacttcgaagtcagaagtcttgtaggttgtggtcactttgctgaagtgcaggtggtaagagagaaagcaaccggggacatctatgctatgaaagtgatgaagaagaaggctttattggcccaggagcaggtttcattttttgaggaagagcggaacatattatctcgaagcacaagcccgtggatcccccaattacagtatgcctttcaggacaaaaatcacctttatctggtcatggaatatcagcctggaggggacttgctgtcacttttgaatagatatgaggaccagttagatgaaaacctgatacagttttacctagctgagctgattttggctgttcacagcgttcatctgatgggatacgtgcatcgagacatcaagcctgagaacattctcgttgaccgcacaggacacatcaagctggtggattttggatctgccgcgaaaatgaattcaaacaagatggtgaatgccaaactcccgattgggaccccagattacatggctcctgaagtgctgactgtgatgaacggggatggaaaaggcacctaccgcctggactgtgactggtggtcagtgggcgtgattgcctatgagatgatttatgggagatcccccttcgcagagggaacctctgccagaaccttcaataacattatgaatttccagcggtttttgaaatttccagatgaccccaaagtgagcagtgactttcttgatctgattcaaagcttgttgtgcggccagaaagagagactgaagtttgaaggtctttgctgccatcctttcttctctaaaattgactggaacaacattcgtaactctcctccccccttcgttcccaccctcaagtctgacgatgacacctccaattttgatgaaccagagaagaattcgtgggtttcatcctctccgtgccagctgagcccctcaggcttctcgggtgaagaactgccgtttgtggggttttcgtacagcaaggcactggggattcttggtagatctgagtctgttgtgtcgggtctggactcccctgccaagactagctccatggaaaagaaacttctcatcaaaagcaaagagctacaagactctcaggacaagtgtcacaaggtatttatttccgcagccggcctccttccttgctccaggatcctcccgtccgtatatgccaagggatccgcccggggccgctgctggctctgagccgcctgatccgtagagagtgaggcgctcctgccttcgctgaagtcgcgcctccagcagctcagagggagatgaattcgggccttgctgttgctgtaaatcctttaaatctaaaccagaggaggccctggatttaaacagtccgtttctcagcatgacccagccagatgtctgcttcttccggcaggtggcctgggtcctcacctgtggctgagatacatcccatctgctttgagtgatgcgaagtctctcttcctagtcttttaaaactcctgcttatgtcactgcggccactgtgttgattacgctcaacgtctcttaacattcactgttcctgcccagaggcaacgctctggaaactaataagtcactgcttgcctgggactcctaagagtgcagacgaataaatatctccttgccctgtcctggatttgtcctctagatctttgcaaggagatggggggggatcaagatggatttgggataaaattaaagtgacgtctgcaaaaacaaaacaaaaacaaaagcaaacaggtgaaaaatgatgattgtggcttcc

SEQ ID NO: 34 - DNA, 3975 bp, Homo sapiens
  misc_feature: Incyte ID No 7395890CB1
agtgtgctggaaagggcggcctcggctgcgccgagagcggagacacaggctcaagatggcagattccgactgaggctgggggggccgagctcgcgcgccgctttcccgtccccgttgccatgaaccgcggacaccccggccccgatggcccccgtgtacgaaggtatggcctcacatgtgcaagttttctcccctcacacccttcaatcaagtgccttctgtagtgtgaagaaactgaaaatagagccgagttccaactgggacatgactgggtacggctcccacagcaaagtgtatagccagagcaagaacatccccctgtcgcagccagccaccacaaccgtcagcacctccttgccggtcccaaacccaagcctaccttacgagcagaccatcgtcttcccaggaagcaccgggcacatcgtggtcacctcagcaagcagcacttctgtcaccgggcaagtcctcggcggaccacacaacctaatgcgtcgaagcactgtgagcctccttgatacctaccaaaaatgtggactcaagcgtaagagcgaggagatcgagaacacaagcagcgtgcagatcatcgaggagcatccacccatgattcagaataatgcaagcggggccactgtcgccactgccaccacgtctactgccacctccaaaaacagcggctccaacagcgagggcgactatcagctggtgcagcatgaggtgctgtgctccatgaccaacacctacgaggtcttagagttcttgggccgagggacgtttgggcaagtggtcaagtgctggaaacggggcaccaatgagatcgtagccatcaagatcctgaagaaccacccatcctatgcccgacaaggtcagattgaagtgagcatcctggcccggttgagcacggagagtgccgatgactataacttcgtccgggcctacgaatgcttccagcacaagaaccacacgtgcttggtcttcgagatgttggagcagaacctctatgactttctgaagcaaaacaagtttagccccttgcccctcaaatacattcgcccagttctccagcaggtagccacagccctgatgaaactcaaaagcctaggtcttatccacgctgacctcaaaccagaaaacatcatgctggtggatccatctagacaaccatacagagtcaaggtcatcgactttggttcagccagccacgtctccaaggctgtgtgctccacctacttgcagtccagatattacagggcccctgagatcatccttggtttaccattttgtgaggcaattgacatgtggtccctgggctgtgttattgcagaattgttcctgggttggccgttatatccaggagcttcggagtatgatcagattcggtatatttcacaaacacagggtttgcctgctgaatatttattaagcgccgggacaaagacaactaggtttttcaaccgtgacacggactcaccatatcctttgtggagactgaagacaccagatgaccatgaagcagagacagggattaagtcaaaagaagcaagaaagtacattttcaactgtttagatgatatggcccaggtgaacatgacgacagatttggaagggagcgacatgttggtagaaaaggctgaccggcgggagttcattgacctgttgaagaagatgctgaccattgatgctgacaagagaatcactccaatcgaaaccctgaaccatccctttgtcaccatgacacacttactcgattttccccacagcacacacgtcaaatcatgtttccagaacatggagatctgcaagcgtcgggtgaatatgtatgacacggtgaaccagagcaaaacccctttcatcacgcacgtggcccccagcacgtccaccaacctgaccatgacctttaacaaccagctgaccactgtccacaaccagccctcagcggcatccatggctgcagtggcccagcggagcatgcccctgcagacaggaacagcccagatttgtgcccggcctgacccgttccagcaagctctcatcgtgtgtccccccggcttccaaggcttgcaggcctctccctctaagcacgctggctactcggtgcgaatggaaaatgcagttcccatcgtcactcaagccccaggagctcagcctcttcagatccaaccaggtctgcttgcccagcaggcttggccaagtgggacccagcagatcctgcttcccccagcatggcagcaactgactggagtggccacccacacatcagtgcagcatgccaccgtgattcccgagaccatggcaggcacccagcagctggcggactggagaaatacgcatgctcacggaagccattataatcccatcatgcagcagcctgcactattgaccggtcatgtgacccttccagcagcacagcccttaaatgtgggtgtggcccacgtgatgcggcagcagccaaccagcaccacctcctcccggaagagtaagcagcaccagtcatctgtgagaaatgtctccacctgtgaggtgtcctcctctcaggccatcagctccccacagcgatccaagcgtgtcaaggagaacacacctccccgctgtgccatggtgcacagtagcccggcctgcagcacctcggtcacctgtgggtggggcgacgtggcctccagcaccacccgggaacggcagcggcagacaattgtcattcccgacactcccagccccacggtcagcgtcatcaccatcagcagtgacacggacgaggaggaggaacagaaacacgcccccaccagcactgtctccaagcaaagaaaaaacgtcatcagctgtgtcacagtccacgactccccctactccgactcctccagcaacaccagcccctactccgtgcagcagcgtgctgggcacaacaatgccaatgcctttgacaccaaggggagcctggagaatcactgcacggggaacccccgaaccatcatcgtgccacccctgaaaacccaggccagcgaagtattggtggagtgtgatagcctggtgccagtcaacaccagtcaccactcgtcctcctacaagtccaagtcctccagcaacgtgacctccaccagcggtcactcttcagggagctcatctggagccatcacctaccggcagcagcggccgggcccccacttccagcagcagcagccactcaatctcagccaggctcagcagcacatcaccacggaccgcactgggagccaccgaaggcagcaggcctacatcactcccaccatggcccaggctccgtactccttcccgcacaacagccccagccacggcactgtgcacccgcatctggctgcagccgctgccgctgcccacctccccacccagccccacctctacacctacactgcgccggcggccctgggctccaccggcaccgtggcccacctggtggcctcgcaaggctctgcgcgccacaccgtgcagcacactgcctacccagccagcatcgtccaccaggtccccgtgagcatgggcccccgggtcctgccctcgcccaccatccacccgagtcagtatccagcccaatttgcccaccagacctacatcagcgcctcgccagcctccaccgtctacactggatacccactgagccccgccaaggtcaaccagtacccttacatataaacactggaggggagggagggagggagggagggagagaatggcccgagggaggagggagagaaggagggaggcgctcctgggaccgtgggcgctggccttttatactgaagatgccgcacacaaacaatgcaaacggggcaggtgcggggggggggggggcagagggcaggggcacggggtcgggacaccagtgaaacttgaaccgggaagtgggaggacgtagagcagagaagagaacatttttaaaaggaagggattaaagagggtgggaaatctatggtttttattttaaaaaagaaaaaggaaaaaaaaaaa

SEQ ID NO: 35 - DNA, 1918 bp, Homo sapiens
  misc_feature: Incyte ID No 7475546CB1
ccgcccgcagcgaggaagcgcccgcgcgggcgcaggcggccgggatggcggggcccggctggggtcccccgcgcctggacggcttcatcctcaccgagcgcctgggcagcggcacgtacgccacggtgtacaaggcctacgccaagaaggacactcgtgaagtggtagccataaagtgtgtagccaagaaaagtctgaacaaggcatcggtggagaacctcctcacggagattgagatcctcaagggcattcgacatccccacattgtgcagctgaaagactttcagtgggacagtgacaatatctacctcatcatggagttttgcgcagggggcgacctgtctcgcttcatccatacccgcaggattctgcctgagaaggtggcgcgtgtcttcatgcagcaattagctagcgccctgcaattcctgcatgaacggaatatctctcacctggatctgaagccacagaacattctactgagctccttggagaagccccacctaaaactggcagactttggtttcgcacaacacatgtccccgtgggatgagaagcacgtgctccgtggctcccccctctacatggcccccgagatggtgtgccagcggcagtatgacgcccgcgtggacctctggtccatgggggtcatcctgtatgaagccctcttcgggcagcccccctttgcctccaggtcgttctcggagctggaagagaagatccgtagcaaccgggtcatcgagctccccttgcggcccctgctctcccgagactgccgggacctactgcagcggctcctggagcgggaccccagccgtcgcatctccttccaggacttctttgcgcacccctgggtggacctggagcacatgcccagtggggagagtctggggcgagcaaccgccctggtggtgcaggctgtgaagaaagaccaggagggggattcagcagccgccttatcactctactgcaaggctctggacttctttgtacctgccctgcactatgaagtggatgcccagcggaaggaggcaattaaggcaaaggtggggcagtacgtgtcccgggctgaggagctcaaggccatcgtctcctcttccaatcaggccctgctgaggcaggggacctctgcccgagacctgctcagagagatggcccgggacaagccacgcctcctagctgccctggaagtggcttcagctgccatggccaaggaggaggccgccggcggggagcaggatgccctggacctgtaccagcacagcctgggggagctactgctgttgctggcagcggagcccccgggccggaggcgggagctgcttcacactgaggttcagaacctcatggcccgagctgaatacttgaaggagcagatgagggaatctcgctgggaagctgacaccctggacaaagagggactgtcggaatctgttcgtagctcttgcacccttcagtgaccctagaagaatgattggacagatgtgagccatctggagcagaggggcactaacccaggctgacgccaagaatgaagtggcccactgcagccctggcgagcaggcttcttggatggacagtgctgagacccccatatcccagagtccccagcctccctcaggttactctgcaccccacagatggtttgatggctgtgctgtatactggaggggagggcaggactctgggagaacagcacttctttcatgagacctttgttactcggtggttactgggtcctgtgcctgtccgttttggggcatgcagccctctatcatttttggctccgagaagagggcaaggggcccccgcagggtacttctgtgcttgccctcgccctgccagcaggcagctgtgcccctggcctggccttcccgggaccccttattccaactcagctcctctttgcactggaatgggg

SEQ ID NO: 36 - DNA, 1689 bp, Homo sapiens
  misc_feature: Incyte ID No 7477076CB1
agtgtgctggaaagctttccagacccctccctcccgctcctgggaaagagagaaaccaccgctgcgggtgggtagagaagcacttggcgcctcggggaggggaccgcgcccgcctcatttgcgccttgcagcactgctggaccaggttacaagatgttcacctaagattgagacctagtgactacatttcctacgggaacaaataaatggtttttcatctcccggagatacattacaaacaaatatggtgctaaaagaactccttacctttctctgactacaatttatttggacatacttttgtattgaagagaggtatacatactgaagctacttgctgtactataggagactctgtcctgtaggatcatggaccatcctagtagggaaaaggatgaaagacaacggacaactaaacccatggcacaaaggagtgcacactgctctcgaccatctggctcctcatcgtcctctggggttcttatggtgggacccaacttcagggttggcaagaagataggatgtgggaacttcggagagctcagattaggtaaaaatctctacaccaatgaatatgtagcaatcaaactggaaccaataaaatcacgtgctccacagcttcatttagagtacagattttataaacagcttggcagtgcaggtgaaggtctcccacaggtgtattactttggaccatgtgggaaatataatgccatggtgctggagctccttggccctagcttggaggacttgtttgacctctgtgaccgaacatttactttgaagacggtgttaatgatagccatccagctgctttctcgaatggaatacgtgcactcaaagaacctcatttaccgagatgtcaagccagagaacttcctgattggtcgacaaggcaataagaaagagcatgttatacacattatagactttggactggccaaggaatacattgaccccgaaaccaaaaaacacataccttatagggaacacaaaagtttaactggaactgcaagatatatgtctatcaacacgcatcttggcaaagagcaaagccggagagatgatttggaagccctaggccatatgttcatgtatttccttcgaggcagcctcccctggcaaggactcaaggctgacacattaaaagagagatatcaaaaaattggtgacaccaaaaggaatactcccattgaagctctctgtgagaactttccagaggagatggcaacctaccttcgatatgtcaggcgactggacttctttgaaaaacctgattatgagtatttacggaccctcttcacagacctctttgaaaagaaaggctacacctttgactatgcctatgattgggttgggagacctattcctactccagtagggtcagttcacgtagattctggtgcatctgcaataactcgagaaagccacacacatagggatcggccatcacaacagcagcctcttcgaaatcaggtggttagctcaaccaatggagagctgaatgttgatgatcccacgggagcccactccaatgcaccaatcacagctcatgccgaggtggaggtagtggaggaagctaagtgctgctgtttctttaagaggaaacggaagaagactgctcagcgccacaagtgaccagtgcctcccaggagtcctcagggcctgggggactctgactcaattgta

SEQ ID NO: 37 - DNA, 1054 bp, Homo sapiens
  misc_feature: Incyte ID No 1874092CB1
ggctggatgctgcgatcccgcaggtgagcgcagcaccctccagccttgcagaagcagccaccatgccagtctctaagtgcccaaaaaagtcggagtccctgtggaaggggtgggaccggaaggcccagaggaacggcctgcggagccaggtatacgctgtgaatggcgactactatgtgggcgagtggaaggacaacgtgaaacacgggaaaggaacacaggtctggaagaagaaaggagccatctatgagggggactggaagtttgggaagcgagacggctacggcaccctcagccttcctgaccaacagacaggaaagtgcaggagagtctactcaggctggtggaaaggtgataagaaatcgggttatgggatccagtttttcggacccaaggagtattatgagggtgactggtgtggcagccagcgcagcgggtggggccgcatgtattacagcaacggcgacatctacgagggacagtgggagaacgacaagcccaacggggagggcatgctgcgcctgaagaacgggaaccgctacgagggctgctgggagagaggcatgaagaacggggcggggcgtttcttccatctggaccacggccagctgtttgaaggcttctgggtggacaatatggccaaatgcgggacgatgatcgactttggccgtgacgaggcccctgagcccactcagttccccattcctgaggtcaaaatcctagaccctgatggtgtgctggcggaggccttggccatgttcaggaagacagaggaaggagattgatgccagagaacacaaacgcttcaggagaaattcaagcctgtgtcacccgatcgctcagaccagtgcggctctggctggaggagtcagcagcagctccaggcatgaccccggcaccctcatagggcccctcactacccccagcactgggtcatttcttgccaataggaaggctggtgcttctctcccaggctgtcctcgggaccctcttcattctctgatctcatcctggaatgcatgagaataaagaataaccaagtggtaaaaaaaaa

SEQ ID NO: 38 - DNA, 3360 bp, Homo sapiens
  misc_feature: Incyte ID No 4841542CB1
agtgtgctggaaaagcgcttcagccctccccgcacagcctactgattcccctgccgcccttgctcacctcctgctcgccatggagtcgctggttttcgcgcggcgctccggccccactccctcggcgcagagctagcccggccgctggcggaagggctgatcaagtcgcccaagcccctaatgaagaagcaggcggtgaagcggcaccaccacaagcacaacctgcggcaccgctacgagttcctggagaccctgggcaaaggcacctacgggaaggtgaagaaggcgcgggagagctcggggcgcctggtggccatcaagtcaatccggaaggacaaaatcaaagatgagcaagatctgatgcacatacggagggagattgagatcatgtcatcgctcaaccaccctcacatcattgccatccatgaagtgtttgagaacagcagcaagatcgtgatcgtcatggagtatgccagccggggcgacctttatgactacatcagcgagcggcagcagctcagtgagcgcgaagctaggcatttcttccggcagatcgtctctgccgtgcactattgccatcagaacagagttgtccaccgagatctcaagctggagaacatcctcttgggtgccaatgggaatatcaagattgctgacttcggcctctccaacctctaccatcaaggcaagttcctgcagacattctgtgggagccccctctatgcctcgccagagattgtcaatgggaagccctacacaggcccagaggtggacagctggtccctgggtgttctcctctacatcctggtgcatggcaccatgccctttgatgggcatgaccataagatcctagtgaaacagatcagcaacggggcctaccgggagccacctaaaccctctgatgcctgtggcctgatccggtggctgttgatggtgaaccccacccgccgggccaccctggaggatgtggccagtcactggtgggtcaactggggctacgccacccgagtgggagagcaggaggctccgcatgagggtgggcaccctggcagtgactctgcccgcgcctccatggctgactggctccggcgttcctcccgccccctcctggagaatggggccaaggtgtgcagcttcttcaagcagcatgcacctggtgggggaagcaccacccctggcctggagcgccagcattcgctcaagaagtcccgcaaggagaatgacatggcccagtctctccacagtgacacggctgatgacactgcccatcgccctggcaagagcaacctcaagctgccaaagggcattctcaagaagaaggtgtcagcctctgcagaaggggtacaggaggaccctccggagctcagcccaatccctgcgagcccagggcaggctgcccccctgctccccaagaagggcattctcaagaagccccgacagcgcgagtctggctactactcctctcccgagcccagtgaatctggggagctcttggacgcaggcgacgtgtttgtgagtggggatcccaaggagcagaagcctccgcaagcttcagggctgctcctccatcgcaaaggcatcctcaaactcaatggcaagttctcccagacagccttggagctcgcggcccccaccaccttcggctccctggatgaactcgccccacctcgccccctggcccgggccagccgaccctcaggggctgtgagcgaggacagcatcctgtcctctgagtcctttgaccagctggacttgcctgaacggctcccagagcccccactgcggggctgtgtgtctgtggacaacctcacggggcttgaggagcccccctcagagggccctggaagctgcctgaggcgctggcggcaggatcctttgggggacagctgcttttccctgacagactgccaggaggtgacagcgacctaccgacaggcactgagggtctgctcaaagctcacctgagtggagtaggcattgccccagcccggtcaggctctcagatgcagctggttgcaccccgaggggagatgccttctcccccacctcccaggacctgcatcccagctcagaaggctgagagggtttgcagtggagccctgagcagggctggatatgggaagtaggcaaatgaaatgcgccaagggttcagtgtctgtcttcagccctgctgaacgaagaggatactaaagagaggggaacgggaatgcccgcgacagagtccacattgcctgtttcttgtgtacatgggggggccacagagacctggaaagagaactctcccagggcccatctcctgcatcccatgaatactctgtacacatggtgccttctaaggacagctccttccctactcattccctgcccaagtggggccagacctctttacacacacattcccgttcctaccaaccaccagaactggatggtggcacccctaatgtgcatgaggcatcctgggaatggtctggagtaacgcttcgttatttttatttttatttttatttatttatttatttttttgagacggagtttcgctcttggtgcccaggctagagtgcaatggcgcgatctcagctcacctcaacctccgcctcccgggttcaagcgattctcctgcctcagcctccctagtagctgggattacaggcgcccgccaccatgcccggctaattttgtatttttagtagagacagggtttctccatgttggtcaggctggtctcaaactcccgacctcaggtgatccacccacctcggcctcccaaagtgctgggattacaggcgtgagccaccgcgccccacctaacccttccttatttagcctaggagtaagagaacacaatctctgtttcttcaatggttctcttcccttttccatcctccaaacctggcctgagcctcctgaagttgctgctgtgaatctgaaagacttgaaaagcctccgcctgctgtgtggacttcatctcaaggggcccagcctcctctggactccaccttggacctcagtgactcagaacttctgcctctaagctgctctaaagtccagactatggatgtgttctctaggccttcaggactctagaatgtccatatttatttttatgttcttggctttgtgttttaggaaaagtgaatcttgctgttttcaataatgtgaatgctatgttctgggaaaatccactatgacatctaagttttgtgtacagagagatatttttgcaactatttccacctcctcccacaaccccccacactccactccacactcttgagtctctttacctaatggtctctacctaatggacctccgtggccaaaaagtaccattaaaaccagaaaggtgattggaaaaaaaaaa

SEQ ID NO: 39 - DNA, 2240 bp, Homo sapiens
  misc_feature: Incyte ID No 7472695CB1
cgggctgaaaagtttctcccggtgcagaattccgggctcagcgacagcctgcgccgagtgtgcgcacctgtcggagacccgccagtccgccggccccggcctgaagttaaatcattttggaaagtgatacagcaaaacaagggttcctccagttttggtggtggaaatgtcacagacatcaagcattggtagtgcagaatctttaatttcactggagagaaaaaaagaaaaaaatatcaacagagatataacctccaggaaagatttgccctcaagaacctcaaatgtagagagaaaagcatctcagcaacaatggggtcggggcaactttacagaaggaaaagttcctcacataaggattgagaatggagctgctattgaggaaatctatacctttggaagaatattgggaaaagggagctttggaatagtcattgaagcgacagacaaggaaacagaaacgaagtgggcaattaaaaaagtgaacaaagaaaaggctggaagctctgctgtgaagttacttgaacgagaggtgaacattctgaaaagtgtaaaacatgaacacatcatacatctggaacaagtatttgaaacgccaaagaaaatgtaccttgtgatggagctttgtgaggatggagaactcaaagaaattctggataggaaagggcatttctcagagaatgagacaaggtggatcattcaaagtctcgcatcagctatagcatatcttcacaataatgatattgtacatagagatctgaaactggaaaatataatggttaaaagcagtcttattgatgataacaatgaaataaacttaaacataaaggtgactgattttggcttagcggtgaagaagcaaagtaggagtgaagccatgctgcaggccacatgtgggactcctatctatatggcccctgaagttatcagtgcccacgactatagccagcagtgtgacatttggagcataggcgtcgtaatgtacatgttattacgtggagaaccaccctttttggcaagctcagaagagaagctttttgagttaataagaaaaggagaactacattttgaaaatgcagtctggaattccataagtgactgtgctaaaagtgttttgaaacaacttatgaaagtagatcctgctcacagaatcacagctaaggaactactagataaccagtggttaacaggcaataaactttcttcggtgagaccaaccaatgtattagagatgatgaaggaatggaaaaataacccagaaagtgttgaggaaaacacaacagaagagaagaataagccgtccactgaagaaaagttgaaaagttaccaaccctggggaaatgtccctgatgccaattacacttcagatgaagaggaggaaaaacagtctactgcttatgaaaagcaatttcctgcaaccagtaaggacaactttgatatgtgcagttcaagtttcacatctagcaaactccttccagctgaaatcaagggagaaatggagaaaacccctgtgactccaagccaaggaacagcaaccaagtaccctgctaaatccggcgccctgtccagaaccaaaaagaaactctaaggttccctccagtgttggacagtacaaaaacaaagctgctcttgttagcactttgatgagggggtaggaggggaagaagacagccctatgctgagcttgtagccttttagctccacagagccccgccatgtgtttgcaccagcttaaaattgaagctgcttatctccaaagcagcataagctgcacgtggcattaaaggacagccaccagtaggcttggcagtgggctgcagtggaaatcaactcaagatgtacacgaaggttttttaggggggcagataccttcaatttaaggctgtgggcacacttgctcatttttacttcaaattcttatgtttaggcacagctatttataggggaaaacaagaggccaaatatagtaatggaggtgccaaataattatgtgcactttgcactagaagactttgttagaaaattactaataaacttgccatacgtattacagcagaagtgcttcagtcattcacatgtgttcgtgagattttaggttgctatagattgtttaagacagcttattttaaatgtagaaaaataggagattttgtaactgcttgccattaacttgctgctaaattcccaatgtattgattaaatcaataaaaaacagatgttactcagcaaaaaaaaaaaaa

SEQ ID NO: 40 - DNA, 3340 bp, Homo sapiens
  misc_feature: Incyte ID No 7477966CB1
cactataacctttctctagggtcaaagagatgatgagtgacaccagcacgttccccaatcacccttcctcccctgctgcatccccatctgggggaaggggagtcatggccagccctgcttgggacaggagcaaagggtggtcccagacccctcagagagctgactttgtctctacccccttgcaggttcatactctcaggccagagaacctcctgctggtgtccaccttggatggaagtctccacgcactaagcaagcagacaggggacctgaagtggactctgagggatgatcccgtcatcgaaggaccaatgtacgtcacagaaatggcctttctctctgacccagcagatggcagcctgtacatcttggggacccaaaaacaacagggattaatgaaactgccattcaccatccctgagctggttcatgcctctccctgccgcagctctgatggggtcttctacacaggccggaagcaggatgcctggtttgtggtggaccctgagtcaggggagacccagatgacactgaccacagagggtccctccaccccccgcctctacattggccgaacacagtatacggtcaccatgcatgacccaagagccccagccctgcgctggaacaccacctaccgccgctactcagcgccccccatggatggctcacctgggaaatacatgagccacctggcgtcctgcgggatgggcctgctgctcactgtggacccaggaagcgggacggtgctgtggacacaggacctgggcgtgcctgtgatgggcgtctacacctggcaccaggacggcctgcgccagctgccgcatctcacgctggctcgagacactctgcatttcctcgccctccgctggggccacatccgactgcctgcctcaggcccccgggacacagccaccctcttctctaccttggacacccagctgctaatgacgctgtatgtggggaaggatgaaactggcttctatgtctctaaagcactggtccacacaggagtggccctggtgcctcgtggactgaccctggcccccgcagatggccccaccacagatgaggtgacactccaagtctcaggagagcgagagggctcacccagcactgctgttagatacccctcaggcagtgtggccctcccaagccagtggctgctcattggacaccacgagctacccccagtcctgcacaccaccatgctgagggtccatcccaccctggggagtggaactgcagagacaagacctccagagaatacccaggccccagccttcttcttggagctattgagcctgagccgagagaaactttgggactccgagctgcatccagaagaaaaaactccagactcttacttggggctgggaccccaagacctgctggcagctagcctcactgctgtcctcctgggagggtggattctctttgtgatgaggcagcaacagccgcaggtggtggagaagcagcaggagacccccctggcacctgcagactttgctcacatctcccaggatgcccagtccctgcactcgggggccagccggaggagccagaagaggcttcagagtccctcaaagcaagcccagccactcgacgaccctgaagctgagcaactcaccgtagtggggaagatttccttcaatcccaaggacgtgctgggccgcggggcaggcgggactttcgttttccggggacagtttgagggacgggcagtggctgtcaagcggctcctccgcgagtgctttggcctggttcggcgggaagttcaactgctgcaggagtctgacaggcaccccaacgtgctccgctacttctgcaccgagcggggaccccagttccactacattgccctggagctctgccgggcctccttgcaggagtacgtagaaaacccggacctggatcgcgggggtctggagcccgaggtcgtgctgcagcagctgatgtctggcctggcccacctgcactctttacacatagtgcaccgggacctgaagccaggaaatattctcatcaccgggcctgacagccagggcctgggcagagtggtgctctcagacttcggcctctgcaagaagctgcctgctggccgctgtagcttcagcctccactccggcatccccggcacggaaggctggatggcgcccgagcttctgcagctcctgccaccagacagtcctaccagcgctgtggacatcttctctgcaggctgcgtgttctactacgtgctttctggtggcagccacccctttggagacagtctttatcgccaggcaaacatcctcacaggggctccctgtctggctcacctggaggaagaggtccacgacaaggtggttgcccgggacctggttggagccatgttgagcccactgccgcagccacgcccctctgccccccaggtgctggcccaccccttcttttggagcagagccaagcaactccagttcttccaggacgtcagtgactggctggagaaggagtccgagcaggagcccctggtgagggcactggaggcgggaggctgcgcagtggtccgggacaactggcacgagcacatctccatgccgctgcagacagatctgagaaagttccggtcctataaggggacatcagtgcgagacctgctccgtgctgtgaggaacaagaagcaccactacagggagctcccagttgaggtgcgacaggcactcggccaagtccctgatggcttcgtccagtacttcacaaaccgcttcccacggctgctcctccacacgcaccgagccatgaggagctgcgcctctgagagcctcttcctgccctactacccgccagactcagaggccaggaggccatgccctggggccacagggaggtgaggtgggctggatgccacacagatggtctccgtgctggctcactgaagagctgagcctgtggctggcctcagaatcaggctgggtgcagtggctcacacctgtaatcccagcattttgggaggctgagtgagaggatcacttgagctcaggagttcgagaccagcctggccaacatggcaacaccccatttctacaaaaaatttgtaaaattagccaggcatggtggcgcacgcctgtagtcccagctgcttgggaggctgaggtgggagaatcacttgagcccaggagttcgaggctgcagtgagccaggatcatgccactgcactccagcctggtccacagagagacactgtcaccccctttcccccacaagactggcagaggctgggcagcctggggctgatgaagcagagatgttcgctggatcccagctcctggcacactgtaaggaaatacaacgaagaggt

SEQ ID NO: 41 - DNA, 2539 bp, Homo sapiens
  misc_feature: Incyte ID No 7163416CB1
cggaggactggcccagcaaggtcccaggtcttccctctcctcagcgcctaagagagaggcccagtgcgggtgaggagtcgcgaggaagaggcggaaggcgccggaaggcaccatgttccgcaagaaaaagaagaaacgccctgagatctcagcgccacagaacttccagcaccgtgtccacacctccttcgaccccaaagaaggcaagtttgtgggcctccccccacaatggcagaacatcctggacacactgcggcgccccaagcccgtggtggacccttcgcgaatcacacgggtgcagctccagcccatgaagacagtggtgcggggcagcgcgatgcctgtggatggctacatctcggggctgctcaacgacatccagaagttgtcagtcatcagctccaacaccctgcgtggccgcagccccaccagccggcggcgggcacagtccctggggctgctgggggatgagcactgggccaccgacccagacatgtacctccagagcccccagtctgagcgcactgacccccacggcctctacctcagctgcaacgggggcacaccagcaggccacaagcagatgccgtggcccgagccacagagcccacgggtcctgcccaatgggctggctgcaaaggcacagtccctgggccccgccgagtttcagggtgcctcgcagcgctgtctgcagctgggtgcctgcctgcagagctccccaccaggagcctcgccccccacgggcaccaataggcatggaatgaaggctgccaagcatggctctgaggaggcccggccacagtcctgcctggtgggctcagccacaggcaggccaggtggggaaggcagccctagccctaagacccgggagagcagcctgaagcgcaggctattccgaagcatgttcctgtccactgctgccacagcccctccaagcagcagcaagccaggccctccaccacagagcaagcccaactcctctttccgaccgccgcagaaagacaaccccccaagcctggtggccaaggcccagtccttgccctcggaccagccggtggggaccttcagccctctgaccacttcggataccagcagcccccagaagtccctccgcacagccccggccacaggccagcttccaggccggtcttccccagcgggatccccccgcacctggcacgcccagatcagcaccagcaacctgtacctgccccaggaccccacggttgccaagggtgccctggctggtgaggacacaggtgttgtgacacatgagcagttcaaggctgcgctcaggatggtggtggaccagggtgacccccggctgctgctggacagctacgtgaagattggcgagggctccaccggcatcgtctgcttggcccgggagaagcactcgggccgccaggtggccgtcaagatgatggacctcaggaagcagcagcgcagggagctgctcttcaacgaggtggtgatcatgcgggactaccagcacttcaacgtggtggagatgtacaagagctacctggtgggcgaggagctgtgggtgctcatggagttcctgcagggaggagccctcacagacatcgtctcccaagtcaggctgaatgaggagcagattgccactgtgtgtgaggctgtgctgcaggccctggcctacctgcatgctcagggtgtcatccaccgggacatcaagagtgactccatcctgctgaccctcgatggcagggtgaagctctcggacttcggattctgtgctcagatcagcaaagacgtccctaagaggaagtccctggtgggaaccccctactggatggctcctgaagtgatctccaggtctttgtatgccactgaggtggatatctggtctctgggcatcatggtgattgagatggtagatggggagccaccgtacttcagtgactccccagtgcaagccatgaagaggctccgggacagccccccacccaagctgaaaaactctcacaaggtcagttggcacacaagggtgcgacctcgcagaccccattcctcctgaggcaaggggaccagaacctgggctcccagcatctcccttccactgaagccacagggtctgggctcctggaaaaggctcctctttccccacacaaaacccgcacctgggtgtggagccgcatctacgcacaagttcgcatgtgcgctccgacaagtcgcctcccacggctgtggcaggagagttgctgcttggcagaagggttgctgcttggcaggcactggtcggaagcccagtggggcccatgagcagggaaagccaggacaccagcaatccctgctgtccagggagggatccggagaagcttcactgagcacaaacccttctaacccgtgtcgggagatccataccatgattcgatgtcctgtccatcacggcgagtcggctcatgctccatcgttgcacaccccgacacagctaagccacagcgttccccttaaagccagtataagtgcatggaagtgtatacatgtaaccctttttgccaaatcggccccaaccccg

SEQ ID NO: 42 - DNA, 2377 bp, Homo sapiens
  misc_feature: Incyte ID No 7472822CB1
agtgtgctggaaagttgaattggaattccctgtggctgtccgaaggcagggtgtccggagagcggtgggctgacctgttcctacaccttgcatcatgccagctttgtcaacgggatctgggagtgacactggtctgtatgagctgttggctgctctgccagcccagctgcagccacatgtggatagccaggaagacctgaccttcctctgggatatgtttggtgaaaaaagcctgcattcattggtaaagattcatgaaaaactacactactatgagaagcagagtccggtgcccattctccatggtgcggcggccttggccgatgatctggccgaagagcttcagaacaagccattaaacagtgagatcagagagctgttgaaactactgtcaaaacccaatgtgaaggctttgctctctgtacatgatactgtggctcagaagaattacgacccagtgttgcctcctatgcctgaagatattgacgatgaggaagactcagtaaaaataatccgtctggtcaaaaatagagaaccactgggagctaccattaagaaggatgaacagaccggggcgatcattgtggccagaatcatgagaggaggagctgcagatagaagtggtcttattcatgttggtgatgaacttagggaagtcaacgggataccagtggaggataaaaggcctgaggaaataatacagattttggctcagtctcagggagcaattacatttaagattatacccggcagcaaagaggagacaccatcaaaagaaggcaagatgtttatcaaagccctctttgactataatcctaatgaggataaggcaattccatgtaaggaagctgggctttctttcaaaaagggagatattcttcagattatgagccaagatgatgcaacttggtggcaagcgaaacacgaagctgatgccaaccccagggcaggcttgatcccctcaaagcatttccaggaaaggagattggctttgagacgaccagaaatattggttcagcccctgaaagtttccaacaggaaatcatctggttttagaaaaagttttcgtcttagtagaaaagataagaaaacaaataaatccatgtatgaatgcaagaagagtgatcagtacgacacagctgacgtacccacatacgaagaagtgacaccgtatcggcgacaaactaatgaaaaatacagactcgttgtcttggttggtcccgtgggagtagggctgaatgaactgaaacgaaagctgctgatcagtgacacccagcactatggcgtgacagtgccccataccaccagagcaagaagaagccaggagagtgatggtgttgaatacattttcatttccaagcatttgtttgagacagatgtacaaaataacaagtttattgaatatggagaatataaaaacaactactacggcacaagtatagactcagttcggtctgtccttgctaaaaacaaagtttgtttgttggatgttcagcctcatacagtgaagcatttaaggacactagaatttaagccctatgtgatatttataaagcctccatcaatagagcgtttgagagaaacaagaaaaaatgcaaagattatttcaagcagagatgaccaaggtgctgcaaaacccttcacagaagaagattttcaagaaatgattaaatctgcacagataatggaaagtcaatatggtcatctttttgacaaaattataataaatgatgacctcactgtggcattcaatgagctcaaaacaacttttgacaaattagagacagagacccattgggtgccagtgagctggttacattcataactaagagaaatttccataattgtctttttctatagagtgcatgatgaaatcaattacagttttggtagtagggtttttaaatctatatcactgtcatagatgtacaatcttggttcaagttgaatgctggttttgtttgtatctttttacagccttatttcaaacgccatgtgttagtataagatccgaaatcaaaatatgcacagtactgtattctaagcaaaacctcaaaccttctcgttgtcttcaatatcgctctatctccaagatgaggctgaaattttcagagagacttagctagaggcttagtatgtatgggagttcagcgcttctgctggtctcaggtgtggctgctgctgtcgagtttgcatgttagctgttgaaggtatcaattcagcagccatgagcagctccagacagacagcgtgagctctgctgtttctgggtggatcatcacagatttagccgggcaggcagtaaggtgtcctcttactattcaaaagtgtagactttctgggggatccactagttctacacgccgcccccgtgacc

SEQ ID NO: 43 - DNA, 2897 bp, Homo sapiens
  misc_feature: Incyte ID No 7477486CB1
atggtggcggggttaactttggggaagggcccggagtccccggatggtgatgtcagcgtgccggagagaaaggacgaggtggcggggggaggcggagaggaggaggaggccgaagagagagggcgccacgcccaatatgtgggcccctatcggctggagaagacgctgggcaaaggacagacagggctggttaaactcggggtccactgcatcacgggtcagaaggtcgccatcaagatcgtgaaccgggagaagctgtcggagtcggtgctgatgaaggtggagcgggagatcgccatcctgaagctcatcgaacacccacatgtcctcaagctccacgacgtctacgagaacaagaaatatttgtacctggttctggagcacgtctcggggggtgagctattcgactacctggtaaagaaggggagactgacgcccaaggaggcccgaaagttcttccgccagattgtgtctgcgctggacttctgccacagctactccatctgccacagagacctaaagcccgagaacctgcttttggatgagaaaaacaacatccgcattgcagacttcggcatggcgtccctgcaggtgggggacagcctcctggagaccagctgcgggtccccccattatgcgtgtccagaggtgattaagggggaaaaatatgatggccgccgggcagacatgtggagctgtggagtcatcctcttcgccctgctcgtgggggctctgccctttgatgacgacaacctccgccagctgctggagaaggtgaaacggggcgtcttccacatgccccacttcattcctccagattgccagagcctcctgaggggaatgatcgaagtggagcccgaaaaaaggctcagtctggagcaaattcagaaacatccttggtacctaggcgggaaacacgagccagacccgtgcctggagccagcccctggccgccgggtagccatgcggagcctgccatccaacggagagctggaccccgacgtcctagagagcatggcatcactgggctgcttcagggaccgcgagaggctgcatcgcgagctgcgcagtgaggaggagaaccaagaaaagatgatatattatctgcttttggatcggaaggagcggtatcccagctgtgaggaccaggacctgcctccccggaatgatgttgaccccccccggaagcgtgtggattctcccatgctgagccgtcacgggaagcggcgaccagagcggaagtccatggaagtcctgagcatcaccgatgccgggggtggtggctcccctgtacccacccgacgggccttggagatggcccagcacagccagagatcccgtagcgtcagtggagcctccacgggtctgtcctccagccctctaagcagcccaaggagtccggtcttttccttttcaccggagccgggggctggagatgaggctcgaggcgggggctccccgacttccaaaacgcagacgctgccttctcggggccccaggggtgggggcgccggggagcagcccccgccccccagtgcccgctccacacccctgcccggccccccaggctccccgcgctcctctggcgggacccccttgcactcgcctctgcacacgccccgggccagtcccaccgggaccccggggacaacaccaccccccagccccggcggtggcgtcgggggagccgcctggaggagtcgtctcaactccatccgcaacagcttcctgggctcccctcgctttcaccggcgcaagatgcaggtccctaccgctgaggagatgtccagcttgacgccagagtcctccccggagctggcaaaacgctcctggttcgggaacttcatctccttggacaaagaagaacaaatattcctcgtgctaaaggacaaacctctcagcagcatcaaagcagacatcgtccatgcctttctgtcgatccccagcctgagtcacagtgtgctgtcacagaccagcttcagggccgagtacaaggccagtggcggcccctccgtcttccaaaagcccgtccgcttccaggtggacatcagctcctctgagggtccagagccctccccgcgacgggacggcagcggaggtggtggcatctactccgtcaccttcactctcatctcgggtcccagccgtcggttcaagcgagtggtggagaccatccaggcacagctcctgagcactcatgaccagccctccgtgcaggccctggcagacgagaagaacggggcccagacccggcctgctggtgccccaccccgaagcctgcagcccccacccggccgcccagacccagagctgagcagctctccccgccgaggcccccccaaggacaagaagctcctggccaccaacgggacccctctgccctgaccccacggggccggggagggaggggacccccctccaccccccttccgtgccccccaactgtgaatctgtaaataaggcccaaggaacatgtcgggaggggggtggacacaaaaaccggccttgccctgcagggatggggctccacaggccgtgcccaactgcgggtggttctaggggaacagggggcgggggagctgtttctattttatttattgattaatttattattttatttattgatcaatctctctgcggggtgcggtgggggagggacgggagctggttggggtggcttagcagatccggacagggccctctgtccctgtgtcgtccccaaccccctcttcccgggcccctcctcccctggtccttccccccacgaccttctgtacggatttgctctccggaaggaattctgataacgcgtgatcctgcctgcgtccgtgtctctgattccgccggcggcaaaaaaaacacaacaccaacaacacaacagggcacaacaaaaaaaa

SEQ ID NO: 44 - DNA, 3361 bp, Homo sapiens
  misc_feature: Incyte ID No 3773709CB1
ggctgagccgggttggggcccgggttgggccgcccggggactctggagcattgggatttgtagcgcgccctctgggtaggcggctgtagcggagangcgtgcgggatcgggatgtcggggctgctcacggacccggagcagagagcgcaggagccgcggtaccccggcttcgtgctggggctggatgtgggcagttctgtgatccgctgccacgtctatgaccgggcggcgcgggtctgcggctccagcgtgcagaaggtagaaaatctttatcctcaaattggctgggtagaaattgatcctgatgttctttggattcaatttgttgccgtaataaaagaagcagtcaaagctgcaggaatacagatgaatcaaattgttggtcttggcatttcaacacagagagcaacttttattacgtggaacaagaaaacaggaaatcattttcacaactttataagttggcaagacttaagagctgttgaacttgtaaaatcttggaataattctcttcttatgaagatatttcacagttcttgccgagtgcttcactttttcactagaagtaaacgactttttacagccagtttgttcactttcacaacccagcagacttctttgagattggtctggattttacagaacttgactgaggtgcaaaaggcagttgaagaagaaaattgctgctttgggactatcgatacctggtggttatataagctcacaaaaggttctgtatatgccacagatttttcaaatgctagtacaactggactttttgacccatatagccacaattttggatcagtggatgaagagatatttggtgtgcctataccaatagttgccttggttgctgaccagcaatcagccatgtttggagagtgctgcttccagacaggtgatgtgaaattaaccatgggaactgggacatttttggatattaacactggaaatagccttcaacagactactggaggcttttatccattaattgggtggaagattgggcaagaagtcgtatgcttagctgaaagcaatgcaggagacactggtactgccataaaatgggctcagcagttagaccttttcacagatgctgctgagactgaaaaaatggccaaaagtttggaggattctgaaggagtttgttttgttccatcttttagtggattacaggctccattaaatgacccctgggcatgtgcctcttttatgggtttgaagccttctaccagtaaataccatcttgtacgagcaatattggagtcaatagctttcagaaacaaacagttatatgagatgatgaagaaagagattcatattcctgtaagaaaaatccgggcagatggaggagtttgtaagaatggttttgtcatgcagatgacttcagacctgattaatgagaatatagacagacctgccgacattgacatgtcatgcctgggtgcagcttctctagctggccttgctgttgggttttggactgacaaggaggaactaaagaaactgagacaaagtgaagtggttttcaagccacagaagaaatgtcaagaatatgaaatgagtctggaaaactgggccaaagcagtgaaacgctccatgaattggtataacaagacataacactaaatgaaatgatcaaaaccataggtagctggtttatgtgacgtgcagatgagatgaagctcagggataacccatatgacaatgactaagaggagaaaattttaaataagcttcataacttaagaagcattgcttttaaaaaaacaaaacggaacaaaaaactcttatttttttcccctaaaccatggtaaggcagcaatacctcaaaactttatatcttctattttgtagcaaattccaaaggacattagtcatttccaaccacattttgacagttatgggtcctcttcctttttatactgggtcagtggtacataggaacataatgatttaccatccaagctaatagttctgggtcaagtaccatgcacatattgttccaaaattatgtgaaacgtatttctttaattctttaagtgggctatttgaagtacatatagctaaaaagaaagaataactgagaaaatgtggaattttgaaacattaatattttatgtttaaagccataatttcctaatattatatccaaatatgagcttaatatgtccctctcagataagcttatgagatagttaatgctttcctttactggtcttaaagacactgccttaatttttccttgttcaaccaaaatctgagcattctttctatgttgaaaacactgaaaaactaattttagttaatgaactagaaagaatattgttttttaagaaacagaaaaatactacttattttccttctcaaataacgtttctttcaaaaacttctggctgaagtataacatgctggtagttaacataaatcttgtctttctcttgttctttatctttctttgttatttagatgcttgtataaatgtcttttgtttttattaagtgcctaattgacagagcttaatttgaagaagtgccctaatttattgaccacttaagaattgcctttattggggtattttatttgttcctgcgtctttttgatgtttgttcagtctactcatccctgtgagtatgtgtgggggacagctgatagaagggaggagagtgtgtctatgctcaggattgccctttagccactcagccagagatccacagggagcaacaaggacagtttcacatgcttagactttcttggaagaaacagtgaggaggagtaagtcgtgagtagtgtcaagctggatgtagaattgtcctaaggcagttgaccccaccttccaacatgttttcactttatttgcccctccctacatttgggttaggttccatttggatttgcagcaataatgactttatttctctcttggtcaggatttggcacataaaatccttttattatagaactagctattttagttacatagtaatgtaactaatggagagatttatagagaattttgtttttgctgtcatatatgtccattttggagacagatatgatagaactagaaattaagttgcatttctgcaagtgccatttgaatgaacttcaagtatcttcttaattattaaattttctgatgaaggcattgtaacaaatatatagtattattaaatctaattaatatttggaaatattaataaataggtattttatttactgtaaaaagtcaaacttcattatgtagataaatcttattcttttcattctttcccctgtttacatcctttttacaaagcttagtcaccaattaaagctttcctatcaaaatcagaaaagaaaaaaagagaagacacac

SEQ ID NO: 45 - DNA, 1662 bp, Homo sapiens
  misc_feature: Incyte ID No 7477204CB1
atggtggacatgggggccctggacaacctgatcgccaacaccgcctacctgcaggcccggaagccctcggactgcgacagcaaagagctgcagcggcggcggcgtagcctggccctgcccgggctgcagggctgcgcggagctccgccagaagctgtccctgaacttccacagcctgtgtgagcagcagcccatcggtcgccgcctcttccgtgacttcctagccacagtgcccacgttccgcaaggcggcaaccttcctagaggacgtgcagaactgggagctggccgaggagggacccaccaaagacagcgcgctgcaggggctggtggccacttgtgcgagtgcccctgccccggggaacccgcaacccttcctcagccaggccgtggccaccaagtgccaagcagccaccactgaggaagagcgagtggctgcagtgacgctggccaaggctgaggccatggctttcttgcaagagcagccctttaaggatttcgtgaccagcgccttctacgacaagtttctgcagtggaaactcttcgagatgcaaccagtgtcagacaagtacttcactgagttcagagtgctggggaaaggtggttttggggaggtatgtgccgtccaggtgaaaaacactgggaagatgtatgcctgtaagaaactggacaagaagcggctgaagaagaaaggtggcgagaagatggctctcttggaaaaggaaatcttggagaaggtcagcagccctttcattgtctctctggcctatgcctttgagagcaagacccatctctgccttgtcatgagcctgatgaatgggggagacctcaagttccacatctacaacgtgggcacgcgtggcctggacatgagccgggtgatcttttactcggcccagatagcctgtgggatgctgcacctccatgaactcggcatcgtctatcgggacatgaagcctgagaatgtgcttctggatgacctcggcaactgcaggttatctgacctggggctggccgtggagatgaagggtggcaagcccatcacccagagggctggaaccaatggttacatggctcctgagatcctaatggaaaaggtaagttattcctatcctgtggactggtttgccatgggatgcagcatttatgaaatggttgctggacgaacaccattcaaagattacaaggaaaaggtcagtaaagaggatctgaagcaaagaactctgcaagacgaggtcaaattccagcatgataacttcacagaggaagcaaaagatatttgcaggctcttcttggctaagaaaccagagcaacgcttaggaagcagagaaaagtctgatgatcccaggaaacatcatttctttaaaacgatcaactttcctcgcctggaagctggcctaattgaacccccatttgtgccagacccttcagtggtttatgccaaagacatcgctgaaattgatgatttctctgaggttcggggggtggaatttgatgacaaagataagcagttcttcaaaaactttgcgacaggtgctgttcctatagcatggcaggaagaaattatagaaacgggactgtttgaggaactgaatgaccccaacagacctacgggttgtgaggagggtaattcatccaagtctggcgtgtgtttgttattgtaa

SEQ ID NO: 46 - DNA, 3225 bp, Homo sapiens
  misc_feature: Incyte ID No 3016969CB1
agtgtgctggaaaggccgccagggaggagcaggccaccctcctggccaaagccccctcattcgagactgccctccggctgcctgcctctggcacccacttggcccctggccacagccactccctggaacatgactctccgagcaccccccgcccctcctcggaggcctgcggtgaggcacagcgactgccttcagccccctccgggggggcccctatcagggacatggggcaccctcagggctccaagcagcttccatccactggtggccacccaggcactgctcagccagagaggccatccccggacagcccttgggggcagccagcccctttctgccaccccaagcagggttctgccccccaggagggctgcagcccccacccagcagttgccccatgccctcctggctccttccctccaggatcttgcaaagaggcccccttagtaccctcaagccccttcttggggacagccccaggcaccccctgcccctgccaaagcaagccccccattggactctaagatggggcctggagacatctctcttcctgggaggccaaaacccggcccctgcagttccccagggtcagcctcccaggcgagctcttcccaagtgagctccctcagggtgggctcctcccaggtgggcacagagcctggcccctccctggatgcggagggctggacccaggaggctgaggatctgtccgactccacacccaccttgcagcggcctcaggaacaggtgaccatgcgcaagttctccctgggtggtcgcgggggctacgcaggcgtggctggctatggcacctttgcctttggtggagatgcagggggcatgctggggcaggggcccatgtgggccaggatagcctgggctgtgtcccagtcggaggaggaggagcaggaggaggccagggctgagtcccagtcggaggagcagcaggaggccagggctgagagcccactgccccaggtcagtgcaaggcctgtgcctgaggtcggcagggctcccaccaggagctctccagagcccaccccatgggaggacatcgggcaggtctccctggtgcagatccgggacctgtcaggtgatgcggaggcggccgacacaatatccctggacatttccgaggtggaccccgcctacctcaacctctcagacctgtacgatatcaagtacctcccattcgagtttatgatcttcaggaaagtccccaagtccgctcagccagagccgccctcccccatggctgaggaggagctggccgagttcccggagcccacgtggccctggccaggtgaactgggcccccacgcaggcctggagatcacagaggagtcagaggatgtggacgcgctgctggcagaggctgccgtgggcaggaagcgcaagtggtcctcgccgtcacgcagcctcttccacttccctgggaggcacctgccgctggacgagcctgcagagctggggctgcgtgagagagtgaaggcctccgtggagcacatctcccggatcctgaagggcaggccggaaggtctggagaaggaggggccccccaggaagaagccaggccttgcttccttccggctctcaggtctgaagagctgggaccgagcgccgacattcctaagggagctctcagatgagactgtggtcctgggccagtcagtgacactggcctgccaggtgtcagcccagccagctgcccaggccacctggagcaaagacggagcccccctggagagcagcagccgtgtcctcatctctgccaccctcaagaacttccagcttctgaccatcctggtggtggtggctgaggacctgggtgtgtacacctgcagcgtgagcaatgcgctggggacagtgaccaccacgggcgtcctccggaaggcagagcgcccctcatcttcgccatgcccggatatcggggaggtgtacgcggatggggtgctgctggtctggaagcccgtggaatcctacggccctgtgacctacattgtgcagtgcagcctagaaggcggcagctggaccacactggcctccgacatctttgactgctgctacctgaccagcaagctctcccggggtggcacctacaccttccgcacggcatgtgtcagcaaggcaggaatgggtccctacagcagcccctcggagcaagtcctcctgggagggcccagccacctggcctctgaggaggagagccaggggcggtcagcccaacccctgcccagcacaaagaccttcgcattccagacacagatccagaggggccgcttcagcgtggtgcggcaatgctgggagaaggccagcgggcgggcgctggccgccaagatcatcccctaccaccccaaggacaagacagcagtgctgcgcgaatacgaggccctcaagggcctgcgccacccgcacctggcccagctgcacgcagcctacctcagcccccggcacctggtgctcatcttggagctgtgctctgggcccgagctgctcccctgcctggccgagagggcctcctactcagaatccgaggtgaaggactacctgtggcagatgttgagtgccacccagtacctgcacaaccagcacatcctgcacctggacctgaggtccgagaacatgatcatcaccgaatacaacctgctcaaggtcgtggacctgggcaatgcacagagcctcagccaggagaaggtgctgccctcagacaagttcaaggactacctagagaccatggctccagagctcctggagggccagggggctgttccacagacagacatctgggccatcggtgtgacagccttcatcatgctgagcgccgagtacccggtgagcagcgagggtgcacgcgacctgcagagaggactgcgcaaggggctggtccggctgagccgctgctacgcggggctgtccgggggcgccgtggccttcctgcgcagcactctgtgcgcccagccctggggccggccctgcgcgtccagctgcctgcagtgcccgtggctaacagaggagggcccggcctgttcgcggcccgcgcccgtgaccttccctaccgcgcggctgcgcgtcttcgtgcgcaatcgcgagaagagacgcgcgctgctgtacaagaggcacaacctggcccaggtgcgctgagggtcgccccggccacacccttggtctccccgctgggggtcgctgcagacgcgccaataaaaacgcacagccgggcgagaaaaa

SEQ ID NO: 47 - DNA, 4772 bp, Homo sapiens
  misc_feature: Incyte ID No 063497CB1
gcggacggacgctcgcctgccggctgaggaaaaagaagcaactaacaaaacactgtgataataaggattattcagtatgcagtttgcaggatatccatgacgacattgaaaatgaattttttgtattcaccagatattcttatatgagaagatctattttaaacagtctaaatattttttcttctgttggaccagcatggcaggatttaagcgagggtatgatggaaagattgctggattatatgatctggataaaaccttgggtcgaggccattttgccgtggttaaacttgccaggcatgtctttacgggtgaaaaggtggcagtaaaagttattgacaagacaaaactggacactctagctactggtcatcttttccaggaagtgagatgcatgaaactagtgcagcatcctaacatcgtccgcctttatgaagttattgacacccagaccaaactatatcttattctagaacttggggatggaggagatatgtttgattatataatgaaacatgaggagggtcttaatgaagacttggccaagaagtattttgctcagatagttcatgctatatcttattgccataaactccatgtggttcacagagacttaaaaccagagaatgtagtcttctttgaaaaacaaggtcttgtaaagttgacagactttgggttcagcaacaaatttcaaccagggaagaagctcactacaagctgtggatctcttgcatattccgctccagaaattctgcttggtgatgagtatgatgcacctgcagtagatatttggagtctgggagtgatccttttcatgttggtgtgtgggcagccgccctttcaagaagccaatgacagtgaaacactgacaatgatcatggattgcaaatatacagtaccatcccatgtgtctaaagagtgtaaagacctaatcacacggatgctacagagagatcccaagagaagggcttctttagaagagattgaaaatcatccttggcttcagggagtggacccttcaccagctacaaagtataacattccccttgtgtcatacaaaaatctctcggaagaggagcacaacagcatcattcagcgcatggtgcttggggacatagcggatcgagacgccattgtagaagccctggaaaccaacaggtataaccatatcacagccacatacttcctgctggctgaaaggatcctgagagaaaagcaagagaaagaaatacagaccagatctgcaagcccgagcaatatcaaggcccagtttaggcagtcatggccaaccaaaattgatgtaccccaggaccttgaggatgacctcacggccactcctttgtcccacgcgactgtccctcagtctcctgctcgggctgctgacagtgtcctcaatggccacaggagcaaaggcctgtgtgactcagctaagaaagatgacctccctgagttggctggaccagcactctctacggtgccacccgcaagcttaaaacccacagccagtgggcggaagtgtctgttcagggtggaagaagatgaagaggaagatgaggaggacaagaaacccatgtccctctcaacacaagtggttttgcgccggaagccatctgtaaccaaccgcctgacatccaggaagagtgcgcccgtcctcaaccagatctttgaggaaggggaatctgacgatgagtttgacatggatgagaatctgcctcccaagttgagcaggttaaagatgaatatagcttctccaggtacagttcacaaacgctaccaccggaggaaaagtcagggccggggctccagctgcagtagttcggagaccagtgatgatgattctgaaagccggcggcggctcgataaagatagcgggttcacctactcctggcaccgacgggatagcagcgaggggccccctggcagtgagggggatggcgggggccagagcaagccaagcaatgccagtggaggggtggacaaggccagccccagtgagaacaatgctggtgggggcagtccctccagcggctcgggtggcaaccccaccaatacatcgggtaccacacgccgctgtgccggccccagcaactccatgcagctggcctctcgcagtgctggggagctcgttgagagcctcaaactcatgagcctctgcctcggctcccagcttcatgggagcaccaagtacattattgatccacagaatggcttgtcattttccagtgtgaaagtccaagagaaatctacgtggaaaatgtgcattagctccacagggaatgcagggcaggtccctgcagtgggcggcataaagtttttctctgaccacatggcagataccaccactgaattggaacggataaagagcaagaacctgaaaaataacgtgctgcagctacctctgtgcgaaaagaccatctctgtgaacatccagcggaaccctaaggaggggctgctgtgcgcatccagcccagccagctgttgccatgtcatctgactgtggccccatctggccgctagcacgcttcctgctcagagcagtgaagaccggctcacttcactgttccatttggttttactattttaaagtgggcgttaggagcaattatttattacctttccatttgttcgcctgatgatgtgacaatgcatggtctttgtgcatgctgctagacacttttctttcccagccgaaaagcctattatgtaatttttacattcataattttaatgtggatgatcaggattaaatcaagatatatatctggaacctcttaaaaatggagcacttagaaatttgttgttctgcacttaacctagagagagaaaaaatgcttttctttgtgaaaaatctgaattcctgtcctgaccttctgtgatgtggaaaccctaggctctgagacacactctctggtgtctgagacagaaccaaagcaataacgttgtgatgcccacaggcctggagccagctagcgaccttgtgccgcccagctgtccatggcccgtgcagagcagaggacagtgagtgtctgcactgagaaccttaaaccacagttgaacatacccacacctgtttgtcttaagctatagtgtaaaaacaaagtttgggctctgaaaatttaactgaaaaagatttccttgtttttgtaataggtgagataaagtacttagatttataaggcagcttcccctgtagtgataaattacaagcagacaatcttattttgtaatgtgatgaagtgatgatgtcttaactctacttagagagtgtatgtctgtctaacagaacaaaaagatgctctgtgtaaattccttcctgtagggcacactgcaggatttccatgtagatagaagaactatagggcctagtacagaaggtgcacacaaatgttggcaaagtcaaaaccccatgaattaaaacctactggaatttggtttttaggagtttggtaattagattatctcttttgttattttcattcagttatatcctttggctcagctagctttgaaattggctgatgaaaaaatatacataaaagggtaaaattcacacatacagcaaacaaaaatgcacaaagcctgcttcgtaacttttttttctggaattgtttttcactttgcctttttctgccaaaacaataatcaaagaactcttgctttaacctattcctgtacaaagactgtttttgaccagataatcatctgttgtggcattctatcttgtaggacactgtatattgcaaattgctgattatggaaggggccagttgctgttttttcatgcagtgccctgggagtcttaaaagcagtgcttagcaacattggtgatagcatgtggctgggacccagggcccttccccactcttcagccccgagtcatgtgtctgaggtgacggactgagacgcatctggtcctgtaattcagagagtgggcacatcaccaaagaactgcattgctgtggtcactgtttcttcaagtacacactgactctgctactttaggataaatatattttactcagaactctgaatttcacagtatacttactaaactaagtaaaaatgatacttaaaatacttattttactttctagacctaggctagatgttttaagctacagctctagttcattgtgatatttataatttgaaagctatgagaatagatgtgtgggtgaagccatagaacatatttgcttgaaattcttgagcagggatcttataaagggccagaaataagatgtgtggttcacatagatagtgagcgtaacatctgtattaaacataggagagaagtttataaagggcattggcaataaactctttgttgcagctgttttccaagcagtgtaaatactttttcctgtgattatgtatagccttggaatggcaccttttaactaacccatatgtgtttggtttcaatggttttttatattcagatgtatatatggtgctcactttaggatcagcagtgttgaccatttatgctgcatagctgtattatagccttattagttgtgtggttgacccttggggtatacaaaaatctctcggaagaggagcacaacagcatcattcagcgcatggtgcttggggacatagcggatcgagacgccattgtagaagccctggaaaccaacaggtataaccatatcacagccacatacttcctgctggctgcaaaggatcctgagagaaaagcaagagaaagaaatacagaccagatctgcaagcccgagcaatatcaaggcccagtttaggcagtcatg

SEQ ID NO: 48 - DNA, 1880 bp, Homo sapiens
  misc_feature: Incyte ID No 1625436CB1
ctcttgctccctcggccgggcggcggtgactgtgcaccgacgtcggcgcgggctgcaccgccgcgtccgcccgcccgccagcatggccaccaccgccacctgcacccgtttcaccgacgactaccagctcttcgaggagcttggcaagggtgctttctctgtggtccgcaggtgtgtgaagaaaacctccacgcaggagtacgcagcaaaaatcatcaataccaagaaattgtctgcccgggatcaccagaaactagaacgtgaggctcggatatgtcgacttctgaaacatccaaacatcgtgcgcctccatgacagtatttctgaagaagggtttcactacctcgtgtttgaccttgttaccggcggggagctgtttgaagacattgtggccagagagtactacagtgaagcagatgccagccactgtatacatcagattctggagagtgttaaccacatccaccagcatgacatcgtccacagggacctgaagcctgagaacctgctgctggcgagtaaatgcaagggtgccgccgtcaagctggctgattttggcctagccatcgaagtacagggagagcagcaggcttggtttggttttgctggcaccccaggttacttgtcccctgaggtcttgaggaaagatccctatggaaaacctgtggatatctgggcctgcggggtcatcctgtatatcctcctggtgggctatcctcccttctgggatgaggatcagcacaagctgtatcagcagatcaaggctggagcctatgatttcccatcaccagaatgggacacggtaactcctgaagccaagaacttgatcaaccagatgctgaccataaacccagcaaagcgcatcacggctgaccaggctctcaagtacccgtgggtctgtcaacgatccacggtggcatccatgatgcatcgtcaggagactgtggagtgtttgcgcaagttcaatgcccggagaaaactgaagggtgccatcctcacgaccatgcttgtctccaggaacttctcagttggcaggcagagctccgcccccgcctcgcctgccgcgagcgccgccggcctggccgggcaagctgccaaaagcctattgaacaagaagtcggatggcggtgtcaagaaaaggaagtcgagttccagcgtgcacctaatgccacagagcaacaacaaaaacagtctcgtaagcccagcccaagagcccgcgcccttgcagacggccatggagccacaaaccactgtggtacacaacgctacagatgggatcaagggctccacagagagctgcaacaccaccacagaagatgaggacctcaaagctgccccgctccgcactgggaatggcagctcggtgcctgaaggacggagctcccgggacagaacagccccctctgcaggcatgcagccccagccttctctctgctcctcagccatgcgaaaacaggagatcattaagattacagaacagctgattgaagccatcaacaatggggactttgaggcctacacgaagatttgtgatccaggcctcacttcctttgagcctgaggcccttggtaacctcgtggaggggatggatttccataagttttactttgagaatctcctgtccaagaacagcaagcctatccataccaccatcctaaacccacacgtccacgtgattggggaggacgcagcgtgcatcgcctacatccgcctcacccagtacatcgacgggcagggtcggcctcgcaccagccagtcagaagagacccgggtctggcaccgtcgggatggcaagtggctcaatgtccactatcactgctcaggggcccctgccgcaccgctgcagtgagctcagccacaggggctttaggagattccag

SEQ ID NO: 49 - DNA, 5747 bp, Homo sapiens
  misc_feature: Incyte ID No 3330646CB1
ggtaggcaggcggctgagccggcggcgggtggcctgcccaacgtgtgctgggtgggagaaggcgaggcggcagcgatgctgtctcttccgtgaggagcgcagaggaggtcgcggcgccggaggccccagaaggctcgaaggcgccgcgggctggggtcggtggcttagggagcccgtccggccatggtggccgcgggtggtggttggcgcggctgcgctgcggcccggggcagtgcggagccgggacagtcgcggcgctgacgcccgcgggccccagctgcagatatgaagcggagccgctgccgcgaccgaccgcagccgccgccgcccgaccgccgggaggatggagttcagcgggcagcggagctgtctcagtctttgccgccgcgccggcgagcgccgcccgggaggcagcggctggaggagcggacgggccccgcggggcccgagggcaaggagcaggatgtagcaactggagttagtcccctgctcttcaggaaactcagtaatcctgacatattttcatccactggaaaagttaaacttcagcgacaactgagtcaggatgattgtaagttatggagaggaaacctggccagctctctatcgggtaagcagctgctccctttgtccagcagtgtacatagcagtgtgggacaggtgacttggcagtcgtcaggagaagcatcaaacctggttcgaatgagaaaccagtcccttggacagtctgcaccttctcttactgctggcctgaaggagttgagccttccaagaagaggcagcttttgtcggacaagtaaccgcaagagcttgattgtgacctctagcacatcacctacactaccacggccacactcaccactccatggccacacaggtaacagtcctttggacagcccccggaatttctctccaaatgcacctgctcacttttcttttgttcctgcccgtaggactgatgggcggcgctggtctttggcctctttgccctcttcaggatatggaactaacactcctagctccactgtctcatcatcatgctcctcacaggaaaagctgcatcagttgcctttccagcctacagctgatgagctgcactttttgacgaagcatttcagcacagagagcgtaccagatgaggaaggacggcagtccccagccatgcggcctcgctcccggagcctcagtcccggacgatccccagtatcctttgacagtgaaataataatgatgaatcatgtttacaaagaaagattcccaaaggccaccgcacaaatggaagagcgactagcagagtttatttcctccaacactccagacagcgtgctgcccttggcagatggagccctgagctttattcatcatcaggtgattgagatggcccgagactgcctggataaatctcggagtggcctcattacatcacaatacttctacgaacttcaagagaatttggagaaacttttacaagatgctcatgagcgctcagagagctcagaagtggcttttgtgatgcagctggtgaaaaagctgatgattatcattgcccgcccagcacgtctcctggaatgcctggagtttgaccctgaagagttctaccaccttttagaagcagctgagggccacgccaaagagggacaagggattaaatgtgacattccccgctacatcgttagccagctgggcctcacccgggatcccctagaagaaatggcccagttgagcagctgtgacagtcctgacactccagagacagatgattctattgagggccatggggcatctctgccatctaaaaagacaccctctgaagaggacttcgagaccattaagctcatcagcaatggcgcctatggggctgtatttctggtgcggcacaagtccacccggcagcgctttgccatgaagaagatcaacaagcagaacctgatcctacggaaccagatccagcaggccttcgtggagcgtgacatactgactttcgctgagaacccctttgtggtcagcatgttctgctcctttgataccaagcgccacttgtgcatggtgatggagtacgttgaagggggagactgtgccactctgctgaagaatattggggccctgcctgtggacatggtgcgtctatactttgcggaaactgtgctggccctggagtacttacacaactatggcatcgtgcaccgtgacctcaagcctgacaacctcctaattacatccatggggcacatcaagctcacggactttggactgtccaaaatgggcctcatgagtctgacaacgaacttgtatgagggtcatattgaaaaggatgcccgggaattcctggacaagcaggtatgcgggaccccagaatacattgcgcctgaggtgatcctgcgccagggctatgggaagccagtggactggtgggccatgggcattatcctgtatgagttcctggtgggctgcgtccctttttttggagatactccggaggagctctttgggcaggtgatcagtgatgagattgtgtggcctgagggtgatgaggcactgcccccagacgcccaggacctcacctccaaactgctccaccagaaccctctggagagacttggcacaggcagtgcctatgaggtgaagcagcacccattctttactggtctggactggacaggacttctccgccagaaggctgaatttattcctcagttggagtcagaggatgatactagctattttgacacccgctcagagcgataccaccacatggactcggaggatgaggaagaagtgagtgaggatggctgccttgagatccgccagttctcttcctgctctccaaggttcaacaaggtgtacagcagcatggagcggctctcactgctcgaggagcgccggacaccacccccgaccaagcgcagcctgagtgaggagaaggaggaccattcagatggcctggcagggctcaaaggccgagaccggagctgggtgattggctcccctgagatattacggaagcggctgtcggtgtctgagtcatcccacacagagagtgactcaagccctccaatgacagtgcgacgccgctgctcaggcctcctggatgcgcctcggttcccggagggccctgaggaggccagcagcaccctcaggaggcaaccacaggagggtatatgggtcctgacacccccatctggagagggggtatctgggcctgtcactgaacactcaggggagcagcggccaaagctggatgaggaagctgttggccggagcagtggttccagtccagctatggagacccgaggccgtgggacctcacagctggctgagggagccacagccaaggccatcagtgacctggctgtgcgtagggcccgccaccggctgctctctggggactcaacagagaagcgcactgctcgccctgtcaacaaagtgatcaagtccgcctcagccacagccctctcactcctcattccttcggaacaccacacctgctccccgttggccagccccatgtccccacattctcagtcgtccaacccatcatcccgggactcttctccaagcagggacttcttgccagcccttggcagcatgaggcctcccatcatcatccaccgagctggcaagaagtatggcttcaccctgcgggccattcgcgtctacatgggtgactccgatgtctacaccgtgcaccatatggtgtggcacgtggaggatggaggtccggccagtgaggcagggcttcgtcaaggtgacctcatcacccatgtcaatggggaacctgtgcatggcctggtgcacacggaggtggtagagctgatcctgaagagtggaaacaaggtggccatttcaacaactcccctggagaacacatccattaaagtggggccagctcggaagggcagctacaaggccaagatggcccgaaggagcaagaggagccgcggcaaggatgggcaagaaagcagaaaaaggagctccctgttccgcaagatcaccaagcaagcatccctgctccacaccagccgcagcctttcttcccttaaccgctccttgtcatcaggggagagtgggccaggctctcccacacacagccacagcctttccccccgatctcccactcaaggctaccgggtgacccccgatgctgtgcattcagtgggagggaattcatcacagagcagctcccccagctccagcgtgcccagttccccagccggctctgggcacacacggcccagctccctccacggtctggcacccaagctccaacgccagtaccgctctccacggcgcaagtcagcaggcagcatcccactgtcaccactggcccacaccccttctcccccacccccaacagcttcacctcagcggtccccatcgcccctgtctggccatgtagcccaggcctttcccacaaagcttcacttgtcacctcccctgggcaggcaactctcacggcccaagagtgcggagccaccccgttcaccactactcaagagggtgcagtcggctgagaaactggcagcagcacttgccgcctctgagaagaagctagccacttctcgcaagcacagccttgacctgccccactctgaactaaagaaggaactgccgcccagggaagtgagccctctggaggtagttggagccaggagtgtgctgtctggcaagggggccctgccagggaagggggtgctgcagcctgctccctcacgggccctaggcaccctccggcaggaccgagccgaacgacgggagtcgctgcagaagcaagaagccattcgtgaggtggactcctcagaggacgacaccgaggaagggcctgagaacagccagggtgcacaggagctgagcttggcacctcacccagaagtgagccagagtgtggcccctaaaggagcaggagagagtggggaagaggatcctttcccgtccagagaccctaggagcctgggcccaatggtcccaagcctattgacagggatcacactggggcctcccagaatggaaagtcccagtggtccccacaggaggctcgggagcccacaagccattgaggaggctgccagctcctcctcagcaggccccaacctaggtcagtctggagccacagaccccatccctcctgaaggttgctggaaggcccagcacctccacacccaggcactaacagcactttctcccagcacttcgggactcacccccaccagcagttgctctcctcccagctccacctctgggaagctgagcatgtggtcctggaaatcccttattgagggcccagacagggcatccccaagcagaaaggcaaccatggcaggtgggctagccaacctccaggatttggaaaacacaactccagcccagcctaagaacctgtctcccagggagcaggggaagacacagccacctagtgcccccagactggcccatccatcttatgaggatcccagccagggctggctatgggagtctgagtgtgcacaagcagtgaaagaggatccagccctgagcatcacccaagtgcctgatgcctcaggtgacagaaggcaggacgttccatgccgaggctgccccctcacccagaagtctgagcccagcctcaggaggggccaagaaccagggggccatcaaaagcatcgggatttggcattggttccagatgagcttttaaagcaaacatagcagttgtttgccatttcttgcactcagacctgtgtaatatatgctcctggaaaccaaaaaaaaaa

SEQ ID NO: 50 - DNA, 3418 bp, Homo sapiens
  misc_feature: Incyte ID No 3562763CB1
gaggtgggacgccccgcggcctacgctcctggcctccccgccttggcctggccgtttaaccgattctttcgcccgcaggtcacaatccaaggtccggctcctccgcgtcccagggccggacggagggatgaggcagggggggcccgggcagcgccgttgctgctccccccgccgcccgcagccatggaaacggggaaggacggcgcccgcagaggtacacaaagcccggagcggaaaatgcgaagcccagtgccgcgggcgcccagcacgaagctgagccggcggcggcgcccgggccatggatccggtggctgccgaggccccgggcgaggccttcctggcgcggcgacggcctgagggcggtggcgggtccgcgcggccgcgttacagcctgttggcggagatcgggcgcggcagctacggcgtggtttatgaggcagtggccgggcgcagcggggcccgggtggcggtcaagaagatccgctgcgacgcccccgagaacgtggagctggcgctggctgaattctgggccctcaccagcctcaagcggcgccaccagaacgtcgtgcagtttgaggagtgcgtcctgcagcgcaatgggttagcccagcgcatgagtcacggcaacaagagctcgcagctttacctgcgcctggtggagacctcgctgaaaggagaaaggatcctgggttatgctgaggagccctgctatctctggtttgtcatggagttctgtgaaggtggagacctgaatcagtatgtcctgtcccggaggccagacccagccaccaacaaaagtttcatgctacagctgacgagcgccattgccttcctgcacaaaaaccatattgtgcacagggacctgaagccagacaacatcctcatcacagagcggtctggcacccccatcctcaaagtggccgactttggactaagcaaggtctgtgctgggctggcaccccgaggcaaagagggcaatcaagacaacaaaaatgtgaatgtgaataagtactggctgtcctcagcctgcggttcggacttctacatggctcctgaagtctgggagggacactacacagccaaggcggacatctttgccctgggcattatcatctgggcaatgatagaaagaatcacttttattgactctgagaccaagaaggagctcctggggacctacattaaacaggggactgagatcgtccctgttggtgaggcgctgctagaaaacccaaagatggagttgcacatcccccaaaaacgcaggacttccatgtctgaggggatcaagcagctcttgaaagatatgttagctgctaacccacaggaccggcctgatgcctttgaacttgaaaccagaatggaccaggtcacatgtgctgcttaaaattcagggctaagcattttgggtgattttaaactaggtcgattcctcgggacccacagtctcaccacgtctcctccagaggacggcagagggtacaggtggtggcctggccggttggcgatctcccgacagctggatccggcaatgtgaagcttttgtttgggtttccccgcttctttttagttttgctttatttttttccttttcttttcttttttttttttcctctttcctttttttaaatttaaaccattgagacttcagaagagcaggacacaatgctgtggacaggcaccaatttctttaaagaaattcaatgtgggcaaggcatatgtgtaaatttcacttttactttttataaggggttagggagctatttttggttttgtccttcactttccctctgtcttccttctttatacttttctcagttctacttatgacacctcatttccctagagaaggcctgcctccccatagggaatctgggggtttcttctggaacggggcgtgaggacacaaggaggcctctgggccacgcctccctaccagatgcaggaactcctggactccttggtgggctggccctggctagcccttgggcctcggagatgatcagaggtgaagaaccgcctggaagaggacaggcccagggtttggccaggagaactaagaaggtctcaactccaggctttgttgtgtttaagctattgagagccccaggccacaccaggacttgcagtggtgggaatccattcctcttctgccctgtgttgcagggaactaggaggtaagggtggagggcgaccatctcgctcttgctggcggtggagcagccatccctgcctttctgttgggaaaaactgttgtgccaaactcttgtgtggaacacagctgggtcttcagcaggcatctgtcactgccgtgaggtcagcgcttctcacctaactgcctcctggattgtcatcttcccagatgtgtcccatagtgtccaggtgtcacagagacggcctgaggccctaagatctggttgtgactttgccatgataacagggtgtcctgaactggctgccgttgtcgtgttctcacagtgaagggcgtgccctgtgtgccggggtccatggtgtcatatgcagtgacacacactgtcaagcgccatttccctcacccctggagacttactgttaggtgcctgccctcagtatagacgtatccaatgggaaaacagcggacctgcccagagcagggaggtgtcgtggaactgggtagaccccctgcagcgttaggggcccatttgtgggctcgccaccttcaggcttccccagccatgacacttcagccccgccacccatgcctgtctgctgcagccatccttgcactctccagcgacactctcgcacctccctaggggaagcttccctccccctgggctgctgctctgagcccgtctgttctccccctgcaagaaggggcaatgctcttgtgttgtccctctgtctggacgcgcctggccactccgaaggcttttcaccccattatggccaaatagtatagggccactggggagggggaagggaatcattttgtgttcatttttgttttctgtttcacctaaaccagcataggattgataggggagacggttggcgggcatttccgtttctatgtgactatgtgaccaaggcagcaggggcttttacctgctaggcggcagtcctttggccctgagaatttgggagagaacagtgcatcaggccaggctcagcaatatgtttgctcacattctttcagccttctctcacccccctcaacaccaaactttcttccttgtgagcagaaggttggctgctgttagcaggatcccacagtgataaccaggcccttcccttcctaagccaaaacccattgtgactgcctgtctctcctgtctctgacttctcaggcagcctcctgagtgcactgagttgtatccgagagggtgggaacagcagcatcccctaattgcagtacacggttccttttccgcccgcc

SEQ ID NO: 51 - DNA, 995 bp, Homo sapiens
  misc_feature: Incyte ID No 621293CB1
cactttgactggccacccgaatctgaaatccagaaccgtctcatggtgccagaggacatctcagagctggagacggctcagaaactgctggagtatcataggaacatcgtcagggtcattccctcctaccccaaaatcctcaaagtcatcagtgctgaccagccatgtgtggacgtcttctaccaggctctgacctatgtccaaagcaaccatcgtactaatgccccgttcaccccgagggtgctgctgctcgggcctgtgggcagtgggaaaagtctgcaggccgccctcctggcccagaaatacaggcttgtcaatgtctgctgtgggcaactgctgaaagaggctgtggcagataggaccacgtttggcgagctcatccagcccttctttgaaaaggagatggcagttcctgacagcctcctcatgaaggtgctgagccagcgcctggaccagcaggactgcatccagaaaggctgggtgctacacggcgtcccgcgggacctcgaccaggcacacctgctgaaccgcctgggctacaatcccaacagggtgtttttcctgaatgtgccatttgattccatcatggagcggctgactctgagaagaattgatccagtcactggggaaaggtaccacctcatgtacaagccacctcccaccatggagatccaggctcgcctcctgcagaacccaaaggatgctgaagagcaggtcaagctgaaaatggacctgttctacaggaactcagctgacttggagcagttgtatgggtcggccatcaccctcaatggggaccaggacccatacacagtcttcgaatacatcgagagtgggatcattaatcccctgcccaagaaaatcccctgatgggttcagagccaggagcgctgccccagggaaagagttaatcccctgcccccagccccccagcctcggcacagctcccctaaaaagccaataaagcctgctggataccaaaaaaaaaaaaaagc

SEQ ID NO: 52 - DNA, 2459 bp, Homo sapiens
  misc_feature: Incyte ID No 7480774CB1
gcggcgtagtggttctgaacatggataggaggggagatgatagctgctggcgtccggtgagcgtgggcagagcgtagtgcgggcagctgcccagcggaaggatcggatgagactggaggcgccgcgaggagggcggcggcggcagccgggacagcagcgacctgggcccggcgcaggggccccggcggggcggccggaggggggggggccctgggcccggacagaggagtccagcctccacagcgagcctgagagggccggcctcgggcctgcgccggggacagagagtccgcaggcagaattctggacagacggacagactgagcccgcggcagctggccttggagtagagaccgagaggcccaagcaaaagacggagccagacaggtccagcctccggacgcatctagaatggagctggtcagagctggagacgacttgtctttggacggagaccgggacagatggcctttggactgatccgcacaggtccgacctccagtttcagcccgaggaggccagcccctggacacagccaggggttcatgggccctggacagagctggaaacgcatgggtcacagactcagccagagagggtcaagtcctgggctgataacctctggacccaccagaacagttccagcctccagactcacccagaaggagcctgtccctcaaaagagccaagtgctgatggctcctggaaagaattgtatactgatggctccaggacacaacaggatattgaaggtccctggacagagccatatactgatggctcccagaaaaaacaggatactgaagcagccaggaaacagcctggcactggtggtttccaaatacaacaggatactgatggctcctggacacaacctagcactgacggttcccagacagcacctgggacagactgcctcttgggagagcctgaggatggcccattagaggaaccagagcctggagaattgctgactcacctgtactctcacctgaagtgtagccccctgtgccctgtgccccgcctcatcattacccctgagacccctgagcctgaggcccagccagtgggacccccctcccgggttgaggggggcagcggcggcttctcctctgcctcttctttcgacgagtctgaggatgacgtggtggccgggggcggaggtgccagcgatcccgaggacaggtctgggagcaaaccctggaagaagctgaagacagttctgaagtattcaccctttgtggtctccttccgaaaacactacccttgggtccagctttctggacatgctgggaacttccaggcaggagaggatggtcggattctgaaacgtttctgtcagtgtgagcagcgcagcctggagcagctgatgaaagacccgctgcgacctttcgtgcctgcctactatggcatggtgctgcaggatggccagaccttcaaccagatggaagacctcctggctgactttgagggcccctccattatggactgcaagatgggcagcaggacctatctggaagaggagctagtgaaggcacgggaacgtccccgtccccggaaggacatgtatgagaagatggtggctgtggaccctggggcccctacccctgaggagcatgcccagggtgcagtcaccaagccccgctacatgcagtggagggaaaccatgagctccacctctaccctgggcttccggatcgagggcatcaagaaggcagatgggacctgtaacaccaacttcaagaagacgcaggcactggagcaggtgacaaaagtgctggaggacttcgtggatggagaccacgtcatcctgcaaaagtacgtggcatgcctagaagaacttcgtgaagctctggagatctcccccttcttcaagacccacgaggtggtaggcagctccctcctcttcgtgcacgaccacaccggcctggccaaggtctggatgatagacttcggcaagacggtggccttgcccgaccaccagacgctcagccacaggctgccctgggctgagggcaaccgtgaggacggctacctctggggcctggacaacatgatctgcctcctgcaggggctggcacagagctgagctgctcagccaccatcaggttaattggatggcgccagtctggctggaggagccctgagatgccatgggaggcctgaggttggccacgggggagctggcctccagggacgggagagattgtgtcatgtgccacacgagaccaacgtggaaaagtctgaagggccttgggagaccaggtagcacctggccccatcatgatgcaggggttttggggacctggaaggaaggtgatgaggcagtgagtcagaaaaaccagaacggggtccccggatctgccgggaaggcttctgaggggctgcccgtgagagcattcagttcacatgtaacagggtagggggatccactagtttataatgccggccgcgtggta